SEQUENCE LISTING (106 sequences)

SEQ ID NO: 1 - DNA, 22 bp, synthetic construct
  source 1..22: mol_type = other DNA
ggcgagaaaactgaaaatcacg

SEQ ID NO: 2 - DNA, 20 bp, synthetic construct
  source 1..20: mol_type = other DNA
cttgccatattccacgtcct

SEQ ID NO: 3 - DNA, 24 bp, synthetic construct
  source 1..24: mol_type = other DNA
agggaatgtcttcccataaaaact

SEQ ID NO: 4 - DNA, 24 bp, synthetic construct
  source 1..24: mol_type = other DNA
gtctaccttttatttgaattcccg

SEQ ID NO: 5 - DNA, 30 bp, synthetic construct
  source 1..30: mol_type = other DNA
aatcaacccgagtgcaatcgaatggaatcg

SEQ ID NO: 6 - DNA, 22 bp, synthetic construct
  source 1..22: mol_type = other DNA
tccattccattcctgtactcgg

SEQ ID NO: 7 - DNA, 20 bp, synthetic construct
  source 1..20: mol_type = other DNA
aatggtacgatctcggctca

SEQ ID NO: 8 - DNA, 20 bp, synthetic construct
  source 1..20: mol_type = other DNA
tagccaggtgtggtgacttg

SEQ ID NO: 9 - DNA, 23 bp, synthetic construct
  source 1..23: mol_type = other DNA
gatgatggtgatgtacagatggg

SEQ ID NO: 10 - DNA, 20 bp, synthetic construct
  source 1..20: mol_type = other DNA
agcctaactgggaggcaccc

SEQ ID NO: 11 - DNA, 24 bp, synthetic construct
  source 1..24: mol_type = other DNA
tgtatacatgtgccatgctggtgc

SEQ ID NO: 12 - DNA, 25 bp, synthetic construct
  source 1..25: mol_type = other DNA
aatgagatcacatggacacaggaag

SEQ ID NO: 13 - DNA, 24 bp, synthetic construct
  source 1..24: mol_type = other DNA
agaggaaggaatgcctcttgcagt

SEQ ID NO: 14 - DNA, 24 bp, synthetic construct
  source 1..24: mol_type = other DNA
ttacaaagcagtattgctgcccgc

SEQ ID NO: 15 - DNA, 21 bp, synthetic construct
  source 1..21: mol_type = other DNA
tcgaacaggaggagcagagag

SEQ ID NO: 16 - DNA, 20 bp, synthetic construct
  source 1..20: mol_type = other DNA
tactagcggttttacgggcg

SEQ ID NO: 17 - DNA, 19 bp, synthetic construct
  source 1..19: mol_type = other DNA
cttcccattctcggccttg

SEQ ID NO: 18 - DNA, 18 bp, synthetic construct
  source 1..18: mol_type = other DNA
tggcccccactctcttct

SEQ ID NO: 19 - DNA, 23 bp, synthetic construct
  source 1..23: mol_type = other DNA
tcaaaggaaagcccatcagacta

SEQ ID NO: 20 - DNA, 20 bp, synthetic construct
  source 1..20: mol_type = other DNA
gccattgcttttggtgtttt

SEQ ID NO: 21 - DNA, 20 bp, synthetic construct
  source 1..20: mol_type = other DNA
aaatggtgctgggaaaactg

SEQ ID NO: 22 - DNA, 18 bp, synthetic construct
  source 1..18: mol_type = other DNA
aagcaagcctgggcaatg

SEQ ID NO: 23 - DNA, 21 bp, synthetic construct
  source 1..21: mol_type = other DNA
acggaatctcgctgattgcta

SEQ ID NO: 24 - DNA, 20 bp, synthetic construct
  source 1..20: mol_type = other DNA
ggtgtcactactcaatacac

SEQ ID NO: 25 - DNA, 17 bp, synthetic construct
  source 1..17: mol_type = other DNA
gcagcctaggtctctgg

SEQ ID NO: 26 - DNA, 19 bp, synthetic construct
  source 1..19: mol_type = other DNA
cctccagtcacaacaactc

SEQ ID NO: 27 - DNA, 19 bp, synthetic construct
  source 1..19: mol_type = other DNA
tattgaagaaggcggctgg

SEQ ID NO: 28 - DNA, 23 bp, synthetic construct
  source 1..23: mol_type = other DNA
cagtcaaaatatggacggatggt

SEQ ID NO: 29 - DNA, 22 bp, synthetic construct
  source 1..22: mol_type = other DNA
attggcaacaccgtattctgct

SEQ ID NO: 30 - DNA, 20 bp, synthetic construct
  source 1..20: mol_type = other DNA
atatcctgcctggatggggt

SEQ ID NO: 31 - DNA, 22 bp, synthetic construct
  source 1..22: mol_type = other DNA
gagcttcttagtcctcctgtgt

SEQ ID NO: 32 - DNA, 20 bp, synthetic construct
  source 1..20: mol_type = other DNA
cgaccactttgtcaagctca

SEQ ID NO: 33 - DNA, 20 bp, synthetic construct
  source 1..20: mol_type = other DNA
aggggtctacatggcaactg

SEQ ID NO: 34 - DNA, 21 bp, synthetic construct
  source 1..21: mol_type = other DNA
gcccagtaatgagtgtccacg

SEQ ID NO: 35 - DNA, 20 bp, synthetic construct
  source 1..20: mol_type = other DNA
agattgcgggtccaagtacc

SEQ ID NO: 36 - DNA, 21 bp, synthetic construct
  source 1..21: mol_type = other DNA
cctgccctcggtatctctaag

SEQ ID NO: 37 - DNA, 21 bp, synthetic construct
  source 1..21: mol_type = other DNA
atatccacgccatttcaccag

SEQ ID NO: 38 - DNA, 23 bp, synthetic construct
  source 1..23: mol_type = other DNA
aatgacagaattcatgaaagcgt

SEQ ID NO: 39 - DNA, 18 bp, synthetic construct
  source 1..18: mol_type = other DNA
ggaggttgtcagagcaga

SEQ ID NO: 40 - DNA, 21 bp, synthetic construct
  source 1..21: mol_type = other DNA
gccatcagtcacttaaacagc

SEQ ID NO: 41 - DNA, 22 bp, synthetic construct
  source 1..22: mol_type = other DNA
gaaactgaagatctcctagcct

SEQ ID NO: 42 - DNA, 20 bp, synthetic construct
  source 1..20: mol_type = other DNA
ctgcacaggttgttctcagc

SEQ ID NO: 43 - DNA, 21 bp, synthetic construct
  source 1..21: mol_type = other DNA
gtttccgaagtggacatcgca

SEQ ID NO: 44 - DNA, 20 bp, synthetic construct
  source 1..20: mol_type = other DNA
catgtcctccaccaagtcct

SEQ ID NO: 45 - DNA, 20 bp, synthetic construct
  source 1..20: mol_type = other DNA
ggcttgtgatggtcaaggtt

SEQ ID NO: 46 - DNA, 21 bp, synthetic construct
  source 1..21: mol_type = other DNA
tcaacacctgtgacttcatgt

SEQ ID NO: 47 - DNA, 18 bp, synthetic construct
  source 1..18: mol_type = other DNA
gtggactgagggcttgta

SEQ ID NO: 48 - DNA, 20 bp, synthetic construct
  source 1..20: mol_type = other DNA
ccagcagtcgtctttgtcac

SEQ ID NO: 49 - DNA, 20 bp, synthetic construct
  source 1..20: mol_type = other DNA
ctctgggttggcacacactt

SEQ ID NO: 50 - DNA, 21 bp, synthetic construct
  source 1..21: mol_type = other DNA
ccagtagtgagaaagggtcgc

SEQ ID NO: 51 - DNA, 20 bp, synthetic construct
  source 1..20: mol_type = other DNA
agggcttggggcaaattgtt

SEQ ID NO: 52 - DNA, 20 bp, synthetic construct
  source 1..20: mol_type = other DNA
gccttcgattctggattcag

SEQ ID NO: 53 - DNA, 21 bp, synthetic construct
  source 1..21: mol_type = other DNA
gtggcattcaaggagtacctc

SEQ ID NO: 54 - DNA, 19 bp, synthetic construct
  source 1..19: mol_type = other DNA
gccgccgtggagatatcat

SEQ ID NO: 55 - DNA, 20 bp, synthetic construct
  source 1..20: mol_type = other DNA
ggcggttttggagaagttga

SEQ ID NO: 56 - DNA, 20 bp, synthetic construct
  source 1..20: mol_type = other DNA
atatctgcggctgatcctgc

SEQ ID NO: 57 - DNA, 20 bp, synthetic construct
  source 1..20: mol_type = other DNA
ttgtaagttcgaatccgggc

SEQ ID NO: 58 - DNA, 23 bp, synthetic construct
  source 1..23: mol_type = other DNA
ccagcattactagtcagaaggaa

SEQ ID NO: 59 - DNA, 21 bp, synthetic construct
  source 1..21: mol_type = other DNA
cacagtgcaatcttgtcatcc

SEQ ID NO: 60 - DNA, 18 bp, synthetic construct
  source 1..18: mol_type = other DNA
cagaactgggcagtaccc

SEQ ID NO: 61 - DNA, 20 bp, synthetic construct
  source 1..20: mol_type = other DNA
aggagacagatggagacaca

SEQ ID NO: 62 - DNA, 21 bp, synthetic construct
  source 1..21: mol_type = other DNA
ttccatctcggcatacagcaa

SEQ ID NO: 63 - DNA, 23 bp, synthetic construct
  source 1..23: mol_type = other DNA
tctttgggtcagagttaaagcca

SEQ ID NO: 64 - DNA, 21 bp, synthetic construct
  source 1..21: mol_type = other DNA
ctcctcagcacccgaagattc

SEQ ID NO: 65 - DNA, 21 bp, synthetic construct
  source 1..21: mol_type = other DNA
gccgtgaaccatttactgtcg

SEQ ID NO: 66 - DNA, 21 bp, synthetic construct
  source 1..21: mol_type = other DNA
tgcagtttaacacgactgtga

SEQ ID NO: 67 - DNA, 22 bp, synthetic construct
  source 1..22: mol_type = other DNA
gaagagttggatactcagcgtc

SEQ ID NO: 68 - DNA, 24 bp, synthetic construct
  source 1..24: mol_type = other DNA
gtggcatccaagatacaaactcaa

SEQ ID NO: 69 - DNA, 21 bp, synthetic construct
  source 1..21: mol_type = other DNA
tccttcctcttgtcacgctca

SEQ ID NO: 70 - DNA, 21 bp, synthetic construct
  source 1..21: mol_type = other DNA
ggtagctgagtttgacttccg

SEQ ID NO: 71 - DNA, 21 bp, synthetic construct
  source 1..21: mol_type = other DNA
gacagcatccctgttgacctt

SEQ ID NO: 72 - DNA, 23 bp, synthetic construct
  source 1..23: mol_type = other DNA
cagcgattgtctggataaactga

SEQ ID NO: 73 - DNA, 22 bp, synthetic construct
  source 1..22: mol_type = other DNA
ggaggtgggtaagctgtatagg

SEQ ID NO: 74 - DNA, 18 bp, synthetic construct
  source 1..18: mol_type = other DNA
cgtgcgagggcgttaata

SEQ ID NO: 75 - DNA, 20 bp, synthetic construct
  source 1..20: mol_type = other DNA
gggtaggtcatggcattgat

SEQ ID NO: 76 - DNA, 21 bp, synthetic construct
  source 1..21: mol_type = other DNA
gtggacgagttgccacatttc

SEQ ID NO: 77 - DNA, 21 bp, synthetic construct
  source 1..21: mol_type = other DNA
tgaccacagccataggattcc

SEQ ID NO: 78 - DNA, 21 bp, synthetic construct
  source 1..21: mol_type = other DNA
taccttccattcatcgcctcc

SEQ ID NO: 79 - DNA, 22 bp, synthetic construct
  source 1..22: mol_type = other DNA
cattgacctggttacactctgg

SEQ ID NO: 80 - DNA, 21 bp, synthetic construct
  source 1..21: mol_type = other DNA
tgtcaaccatagttgtgatcc

SEQ ID NO: 81 - DNA, 22 bp, synthetic construct
  source 1..22: mol_type = other DNA
gcatgttgtaatcaaaggtgag

SEQ ID NO: 82 - DNA, 20 bp, synthetic construct
  source 1..20: mol_type = other DNA
ttggaaacgggatttgtaga

SEQ ID NO: 83 - DNA, 22 bp, synthetic construct
  source 1..22: mol_type = other DNA
cggtttccaacatatgtgtttt

SEQ ID NO: 84 - DNA, 20 bp, synthetic construct
  source 1..20: mol_type = other DNA
ctgccgtctactcctcttgg

SEQ ID NO: 85 - DNA, 20 bp, synthetic construct
  source 1..20: mol_type = other DNA
tttgggacacaatgaaagca

SEQ ID NO: 86 - DNA, 25 bp, synthetic construct
  source 1..25: mol_type = other DNA
gacaccttttttaactatgcgagct

SEQ ID NO: 87 - DNA, 22 bp, synthetic construct
  source 1..22: mol_type = other DNA
tttctcaaggcccaccaatagt

SEQ ID NO: 88 - DNA, 22 bp, synthetic construct
  source 1..22: mol_type = other DNA
ggtcaccgctccaaatatgtag

SEQ ID NO: 89 - DNA, 21 bp, synthetic construct
  source 1..21: mol_type = other DNA
cagtagtccaagttcgtgcga

SEQ ID NO: 90 - DNA, 20 bp, synthetic construct
  source 1..20: mol_type = other DNA
ctgcctcacagctagtgacc

SEQ ID NO: 91 - DNA, 19 bp, synthetic construct
  source 1..19: mol_type = other DNA
ccggcgctggagattattg

SEQ ID NO: 92 - DNA, 18 bp, synthetic construct
  source 1..18: mol_type = other DNA
cggtgctgagctactggc

SEQ ID NO: 93 - DNA, 21 bp, synthetic construct
  source 1..21: mol_type = other DNA
tttgtaccaggagtgtcaagg

SEQ ID NO: 94 - DNA, 21 bp, synthetic construct
  source 1..21: mol_type = other DNA
ggtggaatgagactattgttg

SEQ ID NO: 95 - DNA, 17 bp, synthetic construct
  source 1..17: mol_type = other DNA
aggacatctcccacgtc

SEQ ID NO: 96 - DNA, 21 bp, synthetic construct
  source 1..21: mol_type = other DNA
aaagagataatctggctctgc

SEQ ID NO: 97 - DNA, 20 bp, synthetic construct
  source 1..20: mol_type = other DNA
gctctgagacaatgaacgct

SEQ ID NO: 98 - DNA, 20 bp, synthetic construct
  source 1..20: mol_type = other DNA
tccttcgagtgacaaacacg

SEQ ID NO: 99 - DNA, 20 bp, synthetic construct
  source 1..20: mol_type = other DNA
ccctcaccatcatcctcact

SEQ ID NO: 100 - DNA, 25 bp, synthetic construct
  source 1..25: mol_type = other DNA
tgaggtctttgagggatttgtagtg

SEQ ID NO: 101 - DNA, 24 bp, synthetic construct
  source 1..24: mol_type = other DNA
ggaaccctagtgataaggaatgca

SEQ ID NO: 102 - DNA, 18 bp, synthetic construct
  source 1..18: mol_type = other DNA
gacggtccgctgcaactg

SEQ ID NO: 103 - DNA, 21 bp, synthetic construct
  source 1..21: mol_type = other DNA
cttccctatggccctcattct

SEQ ID NO: 104 - DNA, 22 bp, synthetic construct
  source 1..22: mol_type = other DNA
gaccagcttttgaagggaaatg

SEQ ID NO: 105 - DNA, 21 bp, synthetic construct
  source 1..21: mol_type = other DNA
ctggttgattttgcggtatgg

SEQ ID NO: 106 - DNA, 23 bp, synthetic construct
  source 1..23: mol_type = other DNA
tgacctcaactacatggtctaca

(19) United States
(12) Patent Application Publication
(10) Pub. No.: US 2023/0340127 A1
(43) Pub. Date: Oct. 26, 2023

(54) METHODS AND COMPOSITIONS FOR CANCER TREATMENT BY INHIBITION OF FBXO44

(71) Applicant: Sanford Burnham Prebys Medical Discovery Institute, La Jolla, CA (US)

(72) Inventors: Charles SPRUCK, San Diego, CA (US); Jia Z. SHEN, San Diego, CA (US)

(21) Appl. No.: 18/156,984

(22) Filed: Jan. 19, 2023

Related U.S. Application Data
(60) Provisional application No. 63/301,448, filed on Jan. 20, 2022.

(51) Int. Cl. C07K 16/28; A61K 45/06; A61K 31/713

(52) U.S. Cl. CPC C07K 16/2818; C07K 16/2827; A61K 45/06; A61K 31/713

(57) ABSTRACT

Provided herein are methods and compositions for inhibition of FBXO44 protein or expression in order to enhance immunotherapy treatment for cancer.

### CROSS-REFERENCE

This application claims the benefit of U.S. Provisional Application No. 63/301,448, filed Jan. 20, 2022, which is incorporated herein by reference in its entirety.

### STATEMENT AS TO FEDERALLY SPONSORED RESEARCH

This invention was made with government support under W81XWH-15-1-0383 awarded by the Medical Research and Development Command, and CA030199 awarded by the National Institutes of Health. The government has certain rights in the invention.

### SEQUENCE LISTING

This application contains a Sequence Listing which has been submitted electronically in XML format. The Sequence Listing XML is incorporated herein by reference. Said XML file, created on Jun. 6, 2023, is named 42256-602_201_SL.xml and is 94,405 bytes in size.

### BACKGROUND

Repetitive elements (REs) are normally transcriptionally silenced in somatic cells by repressive epigenetic modifications, which are thought to include DNA methylation and histone modifications such as deacetylation, H3K9me3, and H4K20me3. The reactivation of endogenous retroelements beyond a threshold level of tolerance in cancer cells, such as by treatment with DNA demethylating agents or HDAC or LSD1 inhibitors, can induce viral mimicry responses that augment certain cancer therapies, including immunotherapy. However, these agents can also affect normal cells presenting obvious side effects, limiting their application for cancer therapy due to safety considerations. Hence, there is an urgent need to develop novel tumor cell-selective viral mimicry-inducing strategies for cancer treatment. Therefore, uncovering cancer cell-specific RE silencing mechanisms could provide a basis for the development of a new generation of cancer immunotherapy drugs.

F-box protein FBXO44 plays an essential role in H3K9me3-mediated transcriptional silencing of REs in cancer cells, but not normal cells, thereby providing a therapeutic opportunity for cancer treatment. In various aspects, the disclosure herein provides methods for functional targeting of FBXO44/SUV39H1 induced DNA replication stress and viral mimicry selectively in cancer cells. In various embodiments, the disclosure herein also provides methods and compositions for inhibition of tumor growth and enhanced immunotherapy response.

### SUMMARY

Provided herein is a method of treating a cancer in an individual in need thereof, comprising administering to the individual two components: i) an inhibitor of F-Box protein FBXO44; and ii) a cancer immunotherapy agent. In one embodiment, the cancer immunotherapy agent is an immune checkpoint inhibitor, a cancer vaccine, or a cytokine therapy agent. In some aspects, the immune checkpoint inhibitor is a chemical entity that blocks PD-1, PD-L1, B7-1, B7-2 or CTLA-4. In one embodiment, the immune checkpoint inhibitor is a chemical entity that blocks PD-1, PD-L1 or CTLA-4. In some aspects, the immune checkpoint inhibitor is a chemical entity that blocks PD-1. In some aspects, the chemical entity that blocks PD-1 is an anti-PD-1 antibody. In some aspects, the anti-PD-1 antibody is BioXCell Catalog No. BP0273, BE0273, BP0146, BE0146, BP0033-2, BE0033-2, or a combination thereof. In one embodiment, the anti-PD-1 antibody is Pembrolizumab, Nivolumab, Cemiplimab, or a combination thereof. In one embodiment, the immune checkpoint inhibitor is a chemical entity that blocks PD-L1. In some aspects, the chemical entity that blocks PD-L1 is an anti-PD-L1 antibody. In one aspect, the anti-PD-L1 antibody is Atezolizumab, Avelumab, Durvalumab, or a combination thereof. In some embodiments, the immune checkpoint inhibitor is a chemical entity that blocks CTLA-4. In some embodiments, the chemical entity that blocks CTLA-4 is an anti-CTLA-4 antibody. In some embodiments, the anti-CTLA-4 antibody is Ipilimumab. In one aspect, the cancer is breast cancer, lung cancer, gastric cancer or ovarian cancer. In some embodiments, the cancer is treatment resistant. In some embodiments, the individual previously was treated with another cancer immunotherapy agent. In some embodiments, the individual previously did not respond sufficiently to treatment with the other cancer immunotherapy agent.

In some embodiments, presented herein is a method of treating treatment resistant cancer in an individual in need thereof comprising administering to the individual two components: i) a chemical entity that interferes with F-Box FBXO44 protein synthesis; and ii) a cancer immunotherapy agent. In some embodiments, the chemical entity is a FBXO44 siRNA. In some embodiments, the chemical entity is a human FBXO44 siRNA. In some embodiments, the cancer immunotherapy agent is an immune checkpoint inhibitor, a cancer vaccine, or a cytokine therapy agent. In some embodiments, the immune checkpoint inhibitor is a chemical entity that blocks PD-1, PD-L1, B7-1, B7-2 or CTLA-4. In some embodiments, the immune checkpoint inhibitor is a chemical entity that blocks PD-1, PD-L1 or CTLA-4. In some aspects, the immune checkpoint inhibitor is a chemical entity that blocks PD-1. In some embodiments, the chemical entity that blocks PD-1 is an anti-PD-1 antibody. In some embodiments, the anti-PD-1 antibody is BioXCell Catalog No. BP0273, BE0273, BP0146, BE0146, BP0033-2, BE0033-2, or a combination thereof. In some embodiments, the anti-PD-1 antibody is Pembrolizumab, Nivolumab, Cemiplimab, or a combination thereof. In some embodiments, the immune checkpoint inhibitor is a chemical entity that blocks PD-L1. In some embodiments, the chemical entity that blocks PD-L1 is an anti-PD-L1 antibody. In some embodiments, the anti-PD-L1 antibody is Atezolizumab, Avelumab, Durvalumab, or a combination thereof. In some embodiments, the immune checkpoint inhibitor is a chemical entity that blocks CTLA-4. In some embodiments, the chemical entity that blocks CTLA-4 is an anti-CTLA-4 antibody. In some embodiments, the anti-CTLA-4 antibody is Ipilimumab. In some embodiments, the cancer is breast cancer, lung cancer, gastric cancer or ovarian cancer. In some embodiments, the cancer is treatment resistant. In some embodiments, the individual was previously treated with another cancer immunotherapy agent. In some embodiments, the individual previously did not respond sufficiently to treatment with the other cancer immunotherapy agent.

In some embodiments, provided herein is a method of treating a cancer in an individual in need thereof, comprising determining whether the cancer is resistant to treatment with a first cancer immunotherapy agent; and administering to the individual two components: i) an inhibitor of F-Box protein FBXO44 or SUV39H1; and ii) a second cancer immunotherapy agent, wherein the inhibitor of F-Box protein FBXO44 or SUV39H1 is not 1-benzyl 7-methyl 6-((4-chlorophenyl)sulfonyl)-4,5-dioxo-3,4,5,6-tetrahydropyrrolo[3,2-e]indole-1,7-dicarboxylate or 1-(4-fluorobenzyl) 7-methyl 4,5-dioxo-6-tosyl-3,4,5,6-tetrahydropyrrolo[3,2-e]indole-1,7-dicarboxylate, or a pharmaceutically acceptable salt thereof. In some embodiments, the second cancer immunotherapy agent is an immune checkpoint inhibitor, a cancer vaccine, or a cytokine therapy agent. In some embodiments, the immune checkpoint inhibitor is a chemical entity that blocks PD-1, PD-L1, B7-1, B7-2 or CTLA-4. In some embodiments, the immune checkpoint inhibitor is a chemical entity that blocks PD-1, PD-L1 or CTLA-4. In some embodiments, the immune checkpoint inhibitor is a chemical entity that blocks PD-1. In some embodiments, the chemical entity that blocks PD-1 is an anti-PD-1 antibody. In some embodiments, the anti-PD-1 antibody is BioXCell Catalog No. BP0273, BE0273, BP0146, BE0146, BP0033-2, BE0033-2, or a combination thereof. In some embodiments, the anti-PD-1 antibody is Pembrolizumab, Nivolumab, Cemiplimab, or a combination thereof. In some embodiments, the immune checkpoint inhibitor is a chemical entity that blocks PD-L1. In some embodiments, the chemical entity that blocks PD-L1 is an anti-PD-L1 antibody. In some embodiments, the anti-PD-L1 antibody is Atezolizumab, Avelumab, Durvalumab, or a combination thereof. In some embodiments, the immune checkpoint inhibitor is a chemical entity that blocks CTLA-4. In some embodiments, the chemical entity that blocks CTLA-4 is an anti-CTLA-4 antibody. In some embodiments, the anti-CTLA-4 antibody is Ipilimumab. In some embodiments, the cancer is breast cancer, lung cancer, gastric cancer or ovarian cancer. In some embodiments, the individual previously did not respond sufficiently to treatment with the first cancer immunotherapy agent. In some embodiments, the first cancer immunotherapy agent is Pembrolizumab, Nivolumab, Cemiplimab, Atezolizumab, Avelumab, Durvalumab, Ipilimumab, or a combination thereof. In some embodiments, the first cancer immunotherapy agent is Pembrolizumab, Nivolumab, Cemiplimab, or a combination thereof. In some embodiments, the two components provide a synergistic effect.

In some aspects, provided herein is a method for identifying a compound for use in treating a cancer in an individual in need thereof, comprising screening of one or more test compounds in a F-Box protein FBXO44 inhibition assay. In some embodiments, the method further comprises a prior step of creation and/or provision of a library of test compounds.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

This patent application contains at least one drawing executed in color. Copies of this patent or patent application with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1P show that FBXO44 regulates H3K9me3-mediated transcriptional silencing of REs in cancer cells: FIGS. 1A-1B show schematic and result of the H3K9me3 regulator screen; FIG. 1C shows immunofluorescence (IF) images of chromatin associated H3K9me3 in MDA-MB-231 cells. Scale bar, 20 μm; FIG. 1D shows quantification of H3K9me3 relative intensity (n=15) (top panel) and immunoblots (bottom panel); FIG. 1E shows immunoblot of FBXO44 in cytoplasmic, nuclear, and chromatin fractions; FIG. 1F shows Co-immunoprecipitation (co-IP) of endogenous FBXO44 with Histone H3 in chromatin fractions; FIG. 1G shows immunoblots of the indicated chromatin modifications in chromatin fractions; FIG. 1H shows chromatin immunoprecipitation sequencing (ChIP-seq) results for FBXO44 chromatin binding peaks categorized by chromosome feature; FIG. 1I shows repetitive elements (RE) annotation of FBXO44 chromatin binding peaks; FIG. 1J shows heatmaps of FBXO44 and H3K9me3 ChIP-seq signals; FIG. 1K shows ChIP-seq enrichment profiles of FBXO44 and H3K9me3 peaks; FIG. 1L shows Venn diagram plots of ChIP-seq peaks for FBXO44 and H3K9me3, H3K27me3, and H3K4me3; FIG. 1M shows Visualization of FBXO44 chromatin binding sites and H3K9me3 modifications for a segment of chromosome 8 harboring satellite repeats (SAR). H3K27me3 and H3K4me3 modifications are shown; FIG. 1N shows ChIP analysis of FBXO44 binding to the indicated REs (n=3); FIG. 1O shows ChIP analysis of H3K9me3 levels at the indicated REs (n=3); FIG. 1P shows qRT-PCR analysis of the indicated REs (n=3). Data represent mean±SEM. ns, not significant; *p<0.05; **p<0.01; ***p<0.001; ****p<0.0001 by one-way ANOVA followed by Tukey's multiple comparisons test in FIG. 1D, and two-way ANOVA followed by Tukey's multiple comparisons test in FIG. 1N, Tukey's multiple comparisons test in FIG. 1O, Sidak's multiple comparisons test in FIG. 1P.

FIGS. 2A-2D show that H3K9me3-mediated transcriptional silencing of REs are regulated via FBXO44 in cancer cells: FIG. 2A shows visualization of FBXO44 binding (black) and H3K9me3 modifications (red) on representative genomic regions corresponding to the indicated REs. H3K27me3 and H3K4me3 modifications; FIG. 2B shows Real-Time Quantitative Reverse Transcription PCR (qRT-PCR) analysis of REs, MAVS/STING, IFN-α/β, chemoattractants CCL5, CXCL9, and CXCL10, and PD-L1 in various control and FBXO44 KD cancer cell lines (n=3) (left panels). GAS6 is control. Immunoblots of the indicated proteins are shown (right panels). GAPDH is loading control; FIG. 2C shows analysis of RE transcript levels in RNA-seq data of control and FBXO44 KD MDA-MB-231 cells using RepEnrich2; FIG. 2D shows qRT-PCR analysis of REs, MAVS/STING, IFN-α/β, chemoattractants CCL5, CXCL9, and CXCL10, and PD-L1 in control and LSD1 KD cancer cell lines (n=3). HES1 and EYA2 are positive and negative controls, respectively. Immunoblots of the indicated proteins are shown (insets). GAPDH is loading control. Data represent mean±SEM. ns, not significant; *p<0.05; **p<0.01; ***p<0.001; ****p<0.0001 by two-way ANOVA followed by Sidak's multiple comparisons test in FIG. 2B, and Dunnett's multiple comparisons test in FIG. 2D.

FIGS. 3A-3M shows that FBXO44 recruits SUV39H1, CRL4RBBP4/7, and Mi-2β/NuRD to REs: FIG. 3A shows STRING network plot for interactions among FBXO44-interacting proteins; FIG. 3B shows co-immunoprecipitation (co-IP) of endogenous FBXO44 with SUV39H1 and components of CRL4 and Mi-2β/NuRD; FIG. 3C shows immunofluorescence (IF) images of chromatin associated H3K9me3 in MDA-MB-231 cells (top panel). Scale bar, 10 μm. Quantification is shown (n=15) (bottom panel); FIG. 3D shows chromatin immunoprecipitation (ChIP) analysis of H3K9me3 levels at indicated REs (n=3); FIG. 3E shows Real-Time Quantitative Reverse Transcription PCR (qRT-PCR) analysis of RE transcripts (n=3); FIG. 3F shows Co-IP of endogenous SUV39H1 with CUL4B and DDB1; FIG. 3G show Co-IP of endogenous CUL4B with DDB1; FIGS. 3H-3I show Co-IP of endogenous FBXO44 with SUV39H1 and GATAD2A, respectively; FIG. 3J shows ChIP analysis of H3K9me3 levels at the indicated REs (n=3); FIG. 3K shows immunoblots of the indicated proteins in chromatin fractions; FIGS. 3L, 3M show ChIP analysis of binding of the indicated Flag-tagged proteins to various REs (n=3). Data represent mean±SEM. ns, not significant; **p<0.01; ***p<0.001; ****p<0.0001 by one-way ANOVA followed by Tukey's multiple comparisons test in FIG. 3C, and two-way ANOVA followed by Sidak's multiple comparisons test in FIGS. 3D and 3L, Tukey's multiple comparisons test in FIGS. 3E, 3J, and 3M.

FIGS. 4A-4O show interaction of FBXO44, SUV39H1, CRL4RBBP4/7, and Mi-2/NuRD on REs region of chromatin: FIG. 4A shows co-immunoprecipitation (co-IP) of endogenous FBXO44 with CRL4 components CUL4A/CUL4B and DCAFs RBBP4/RBBP7 in MDA-MB-231 cells. IgG is control. Input is 5% of WCE used for IP; FIG. 4B shows chromatin immunoprecipitation (ChIP) analysis of CUL1 and CUL4B binding to the indicated REs (n=3). ChIP was performed with anti-CUL1 or anti-CUL4B antibody. Gapdh is control; FIG. 4C shows Real-Time Quantitative Reverse Transcription PCR (qRT-PCR) analysis of the indicated REs in control, CUL1 KD, and CUL4B KD MDA-MB-231 cells (n=3). GAS6 and EYA2 are controls. Immunoblots of the indicated proteins are shown (inset). GAPDH is loading control; FIG. 4D shows Co-IP of Myc-FBXO44 (top panel) or Myc-AF-FBXO44 (bottom panel) with HA-CUL1 in MBA-MB-231 cells; FIG. 4E shows immunoblots of Myc-FBXO44 and Myc-AF-FBXO44 in cytoplasmic, nuclear, and chromatin fractions of MDA-MB-231 cells. α-Tubulin, GAPDH, and ORC2 are fractionation controls; FIG. 4F shows Co-IP of Myc-FBXO44 or Myc-AF-FBXO44 with Flag-Histone H3.1 in MDA-MB-231 cells; FIGS. 4G-41I show Co-IP of Myc-FBXO44 or Myc-AF-FBXO44 with CRL4 components Flag-CUL4B in top panel of FIG. 4G or Flag-DDB1 in bottom panel of FIG. 4G, and Myc-AF-FBXO44 with DCAFs Flag-RBBP4/7 in FIG. 4H in MBA-MB-231 cells; FIGS. 4I-4J show Co-IP of Myc-FBXO44 or Myc-AF-FBXO44 with Flag-GATAD2B in FIG. 4I or Flag-SUV39H1 in FIG. 4J in MDA-MB-231 cells. * indicates IgG light chain in FIG. 4J; FIGS. 4K-4N show ChIP analysis of binding of the indicated Flag-tagged proteins to various REs in MDA-MB-231 cells transfected with non-targeting or RBBP4+7 siRNAs in FIG. 4K CUL4B siRNA in FIG. 4L, GATAD2A+B siRNAs in FIG. 4M, or SUV39H1 siRNA in FIG. 4N (n=3). ChIP was performed with anti-Flag antibody; FIG. 4O shows ChIP analysis of H2AK119ub levels at the indicated REs in control and FBXO44 KD MDA-MB-231 cells (n=3). Gash and Gapdh are controls. ChIP was performed with anti-H2AK119ub antibody. Data represent mean±SEM. ns, not significant; *p<0.05; ***p<0.001; ****p<0.0001 by two-way ANOVA followed by Tukey's multiple comparisons test in FIG. 4B, Dunnett's multiple comparisons test in FIG. 4C, and Sidak's multiple comparisons test in FIGS. 4K-4O.

FIGS. 5A-5J show that FBXO44 promotes RE silencing post-DNA replication: FIG. 5A shows immunofluorescence (IF) images of FBXO44 (middle panel) in HeLa cells synchronized at the indicated cell cycle phases (left panel). Scale bar, 10 μm. Quantification of cells with FBXO44 nuclear localization is shown (n=5) (right panel); FIG. 5B shows co-immunoprecipitation (co-IP) of endogenous FBXO44 with Flag-Histone H3.1 or H3.3; FIG. 5C shows accelerated native isolation of protein on nascent DNA (aniPOND) analysis of FBXO44 chromatin binding. Schematic of protocol (left panel) and immunoblots of FBXO44, DNA replication fork protein PCNA, and Histone H3 (control) are shown (right panel); FIG. 5D shows in vitro binding assay using recombinant FBXO44 and H3K9me1-, H3K9me3-, or un-modified nucleosomes; FIG. 5E shows aniPOND analysis of FBXO44 binding to H3K9me3-modified nucleosomes. Schematic of protocol (left panel) and immunoblots are shown (right panel); FIG. 5F shows model of FBXO44 regulation of H3K9me3-mediated RE silencing post-DNA replication; FIG. 5G shows flow cytometry analysis of cell cycle (left panel) and quantification (n=3) (right panel); FIG. 5H shows IF images of EdU incorporation in DNA of MDA-MB-231 cells (left panel) and quantification (n=5) (right panel). Scale bar, 20 μm; FIG. 5I shows Immunoblots of DNA replication checkpoint and DNA damage response (DDR) proteins. * indicates p-RPA32T21; FIG. 5J shows chromatin immunoprecipitation (ChIP) analysis of γH2AX levels at the indicated REs (n=3). Data represent mean±SEM. ns, not significant; *p<0.05; ***p<0.001; ****p<0.0001 by one-way ANOVA followed by Tukey's multiple comparisons test in FIG. 5A, Dunnett's multiple comparisons test in FIG. 5H, and two-way ANOVA followed by Dunnett's multiple comparisons test in FIG. 5G, Tukey's multiple comparisons test in FIG. 5J.

FIGS. 6A-6B show that FBXO44/SUV39H1 inhibition promotes DNA replication stress and DSBs in cancer cells: FIG. 6A shows immunofluorescence (IF) images of p-RPA32T21 in control, FBXO44 KD, and SUV39H1 KD MDA-MB-231 cells (left panel). DNA stained with DAPI. Scale bar, 10 μm. Fifteen cells from 3 different fields (5 for each) were randomly selected and p-RPA32T21 quantified using ImageJ (n=15) (right panel); FIG. 6B shows IF images of γH2AX in MDA-MB-231 cells transfected with the indicated siRNAs (left panel). DNA stained with DAPI. Scale bar, 10 μm. Fifteen cells from 3 different fields (5 for each) were randomly selected and γH2AX quantified using ImageJ (n=3) (right panel). Data represent mean±SEM. ****p<0.0001 by one-way ANOVA followed by Tukey's multiple comparisons test in FIGS. 6A-6B.

FIGS. 7A-7M show that FBXO44 inhibition activates RIG-I/MDA5-MAVS and cGAS-STING antiviral pathways and IFN signaling and enhances cancer cell immunogenicity: FIGS. 7A-7B show immunofluorescence (IF) images of MDA-MB-231 cells (left panels) and quantification of relative intensity (right panels) of dsRNA in FIG. 7A and dsDNA in FIG. 7B as indicated by arrows (n=15). Scale bar, 10 μm; FIGS. 7C-7D show Real-Time Quantitative Reverse Transcription PCR (qRT-PCR) analysis of the indicated REs (n=3) on right panels. Protocols are shown on left panels; FIG. 7E shows qRT-PCR analysis (n=3) (left panel). Immunoblots of the indicated proteins (right panel); FIG. 7F shows qRT-PCR analysis (n=3) (middle and right panels). Schematic of protocol (left panel); FIG. 7G shows IF images of cGAS and γH2AX positive micronuclei (left panel). DNA stained with DAPI. Scale bar, 5 μm. Quantification is shown (n=5) (right panel); FIG. 7H shows Pathway enrichment map for significantly enriched gene sets in GSEA of FBXO44 KD RNA-seq. p<0.01; false discovery rate (FDR)<0.1; FIG. 7I shows GSEA enrichment plots for selected gene sets in FBXO44 KD RNA-seq; FIG. 7J shows ELISA quantification of IFN-β, CCL5, and CXCL10 (n=3); FIG. 7K shows qRT-PCR analysis of IFN-β (n=3). Day is time post-KD; FIG. 7L shows GSEA analysis of immune-stimulatory pathways; FIG. 7M shows heatmap of representative genes from RNA-seq data. Data represent mean±SEM. ns, not significant; nd, not detected; *p<0.05; **p<0.01; ***p<0.001; ****p<0.0001 by one-way ANOVA followed by Tukey's multiple comparisons test in FIGS. 7A, 7B, and 7F, Dunnett's multiple comparisons test in FIG. 7G, two-way ANOVA followed by Tukey's multiple comparisons test in FIGS. 7C and 7K, Dunnett's multiple comparisons test in FIG. 7D, Sidak's multiple comparisons test in FIG. 7E, and unpaired Student's t-test in FIG. 7J.

FIGS. 8A-8F show that activation of RIG-I/MDA5-MAVS and cGAS-STING antiviral pathways and IFN signaling and enhancement of cancer cell immunogenicity are results of FBXO44 inhibition: FIG. 8A shows Real-Time Quantitative Reverse Transcription PCR (qRT-PCR) analysis of the indicated RE transcripts (right panel) in total RNA isolated from control or FBXO44 KD MDA-MB-231 cells and treated with RNase A (n=3) (left panel; FIG. 8B shows qRT-PCR analysis of REs, MAVS/STING, IFN-α/β, ISGs, and PD-L1 in control and SUV39H1 KD MDA-MB-231 cells. EYA2 and GAS6 are controls. Immunoblots are shown (inset). GAPDH is control; FIG. 8C shows heatmap of differentially expressed ISGs identified by RNA-seq (control KD vs. FBXO44 KD); FIG. 8D shows qRT-PCR analysis of IFN-β in MDA-MB-231 cells transfected with the indicated siRNAs; FIG. 8E shows IF images (left panels) and quantification of relative intensity (right panel) of CTA SSX1 and NKG2D ligand ULBP2 in control and FBXO44 KD MDA-MB-231 cells. DNA stained with DAPI. Scale bar, 10 μm. Fifteen cells from 3 different fields (5 for each) were randomly selected and quantified using ImageJ (n=15); FIG. 8F shows Fluorescence-Activated Cell Sorting (FACS) plots of SSX1 and ULBP2 surface expression on control and FBXO44 KD MDA-MB-231 cells in FIG. 8E. Data represent mean±SEM. ns, not significant; *p<0.05; **p<0.01; ***p<0.001; ****p<0.0001 by two-way ANOVA followed by Dunnett's multiple comparisons test in FIGS. 8A-8B, Tukey's multiple comparisons test in FIG. 8D, and one-way ANOVA followed by Tukey's multiple comparisons test in FIG. 8E.

FIGS. 9A-9H show that FBXO44/SUV39H1 inhibition selectively decreases cancer cell proliferation and viability in vitro: FIG. 9A shows growth curves of the indicated cancer cell lines and patient-derived glioblastoma cultures (n=3); FIG. 9B shows representative flow cytometry analysis of apoptotic (Annexin V+) cells (left panel). Quantification is shown (n=3) (right panel); FIG. 9C shows representative images (left panel) and quantification (right panel) of tumorspheres (n=3) at day 14. Scale bar, 100 μm; FIG. 9D shows representative images (left panel) and quantification (n=3) (right panel) of migration and invasion analyses of MDA-MB-231 cells. Scale bar, 50 μm; FIG. 9E shows growth curves (n=3) (left panel) and immunoblots of FBXO44, MAVS, and STING (right panel); FIG. 9F shows flow cytometry analysis of cell cycle (left panel) and quantification (n=3) (right panel); FIG. 9G shows viabilities of the indicated cells after treatment with F5446 for 48 hrs (n=3); FIG. 9H shows growth curves of HMECs and astrocytes (n=3) (left panels) and immunoblots of the indicated proteins (right panels). Data represent mean±SEM. ns, not significant; ***p<0.001; ****p<0.0001 by one-way ANOVA followed by Dunnett's multiple comparisons test in FIGS. 9B and 9D, and two-way ANOVA followed by Dunnett's multiple comparisons test in FIG. 9A, Sidak's multiple comparisons test in FIG. 9C, Tukey's multiple comparisons test in FIGS. 9E, 9F, 9G, and 9H.

FIGS. 10A-10L show that inhibition of FBXO44/SUV39H1 selectively decreases cancer cell proliferation and viability in vitro: FIG. 10A shows GSEA enrichment plots for selected gene sets by RNA sequencing (RNA-seq) (FBXO44 KD vs. control KD). NES and p values are shown; FIG. 10B shows flow cytometry analysis of breast CSCs (CD24low/− CD44+) in the indicated control and FBXO44 KD cell lines. The % of CSCs is indicated in each sub-figure; FIG. 10C shows flow cytometry analysis of apoptotic (Annexin V+) MDA-MB-231 cells transfected with the indicated siRNAs (left panel). Quantification of the % of Annexin V+ cells is shown (n=3) (right panel); FIG. 10D shows immunofluorescence (IF) images of γH2AX in MDA-MB-231 cells transfected with the indicated siRNAs (left panel). DNA stained with DAPI. Scale bar, 10 μm. Fifteen cells from 3 different fields (5 for each) were randomly selected and γH2AX intensity quantified using ImageJ (n=15) (right panel); FIG. 10E shows IF images of chromatin associated H3K9me3 modifications in MDA-MB-231 cells treated with vehicle or F5446 for 48 hrs (left panel). DNA stained with DAPI. Scale bar, 10 μm. Fifteen cells from 3 different fields (5 for each) were randomly selected and H3K9me3 intensity quantified using ImageJ (n=15) (right panel); FIGS. 10F-10G show Real-Time Quantitative Reverse Transcription PCR (qRT-PCR) analysis of endogenous retroelements, IFN-α/β, ISGs and PD-L1 in vehicle or F5446-treated MDA-MB-231 in FIG. 10F or 4T1 cells in FIG. 10G (n=3); FIG. 10H shows qRT-PCR analysis of REs, MAVS/STING, and IFN-α/β in control and FBXO44 KD HMECs and astrocytes (n=3); FIG. 10I shows IF images of γH2AX in control and FBXO44 KD HMECs and astrocytes (left panels). DNA stained with DAPI. Scale bar, 10 μm. Fifteen cells from 3 different fields (5 for each) were randomly selected and γH2AX intensity quantified using ImageJ (n=15) (top right panels). Immunoblots of the indicated proteins is shown (bottom right panels); FIG. 10J shows chromatin immunoprecipitation (ChIP) analysis of H3K9me3 levels at the indicated REs in HMECs, astrocytes, and cancer cell lines (n=3). ChIP was performed with anti-H3K9me3 antibody; FIGS. 10K-10L show ChIP analysis of FBXO44 in FIG. 10K and SUV39H1 in FIG. 10L binding to the indicated REs in HMECs, astrocytes, and cancer cell lines (n=3). ChIP was performed with anti-FBXO44 in FIG. 10K or anti-SUV39H1 in FIG. 10L antibody. Data represent mean±SEM. ns, not significant; *p<0.05; **p<0.01; ***p<0.001; ****p<0.0001 by one-way ANOVA followed by Tukey's multiple comparisons test in FIGS. 10A, 10B, and 10I, unpaired Student's t-test in E, two-way ANOVA followed by Sidak's multiple comparisons test in FIGS. 10F-10H, and Tukey's multiple comparisons test in FIGS. 10J-10L.

FIGS. 11A-11L show that FBXO44/SUV39H1 inhibition decreases tumor growth, enhances antitumor immune response, and overcomes resistance to ICB therapy: FIG. 11A shows representative bioluminescent images of immunodeficient mice at the indicated days following intracardiac injection of MDA-MB-231-luc cells expressing non-targeting or 2 different FBXO44 shRNAs (left panel). Plot of total body radiance at the indicated days is shown (n=8) (right panel); FIG. 11B shows Total radiance of the indicated tissues isolated from immunodeficient mice in FIG. 11A 28 days post-intracardiac injection (n=3); FIG. 11C shows representative images of metastatic lesions (arrows) in lungs of mice in FIG. 11A. Scale bar, 1 mm; inset scale bar, 200 μm; FIG. 11D shows growth curves for MDA-MB-231-luc-derived mammary tumors expressing the indicated shRNAs in immunocompromised mice (n=5); FIGS. 11E-11F show representative bioluminescence images in FIG. 11E top panel, and images of dissected mammary tumors in FIG. 11F for mice in FIG. 11D at day 25. Quantification of total body radiance is shown (n=5) in FIG. 11E bottom panel; FIG. 11G shows qRT-PCR analysis of endogenous retroelements, IFN-α/β, chemoattractants, and PD-L1 in 4T1 cells expressing non-targeting, FBXO44, or SUV39H1 shRNA (n=3); FIG. 11H shows representative images of syngeneic immunocompetent mice with 4T1-derived mammary tumors expressing the indicated shRNAs at day 22 post-transplantation (left panel). Tumor growth curves are shown (n=4) (right panel); FIG. 11I shows flow cytometry quantification of the indicated infiltrating immune cells in 4T1-derived mammary tumors in FIG. 11H harvested at day 22 (n=4); FIG. 11J shows flow cytometry analysis of PD-L1 and MHC-I (H-2Kd) surface expression on 4T1 tumor cells in (H). shCtrl (n=4), shFBXO44 (n=4), shMAVS+shSTING (n=4), shFBXO44+shMAVS+shSTING (n=4); FIG. 11K shows growth curves for 4T1-derived mammary tumors expressing the indicated shRNAs in syngeneic immunocompetent mice treated with anti-PD-1 antibody (n=4); FIG. 11L shows survival curves for mice in FIG. 11K. Data represent mean±SEM. ns, not significant; *p<0.05; **p<0.01; ***p<0.001; ****p<0.0001 by two-way ANOVA followed by Tukey's multiple comparisons test in FIGS. 11A, 11B, 11D, 11G, 11H, and 11K, one-way ANOVA followed by Tukey's multiple comparisons test in FIGS. 11E, 11F, 11I, and 11J, and log-rank test in FIG. 11L.

FIGS. 12A-12O show that inhibition of FBXO44/SUV39H1 decreases tumor growth, enhances anti-tumor immune response, and overcomes resistance to ICB therapy in mouse model: FIG. 12A shows representative images of syngeneic immunocompetent mice at day 22 post-transplantation (top panel) and tumor growth curves (n=6) (bottom panel); FIGS. 12B-12C show flow cytometry quantification of the indicated infiltrating immune cells in tumors in FIG. 12A (n=6); FIG. 12D shows representative immunohistochemistry (IHC) images of indicated infiltrating immune cells in tumors in FIG. 12A (left panel) and quantification (n=6) (right panels). Scale bar, 50 μm; FIG. 12E shows flow cytometry analysis of PD-L1 and MHC-I (H-2Kd) surface expression on 4T1 tumor cells in FIG. 12A. shCtrl (n=4), shFBXO44 (n=6), and shSUV39H1 (n=4); FIG. 12F shows growth curves of tumors in syngeneic immunocompetent mice (n=5); FIG. 12G shows survival curves for mice in FIG. 12F; FIG. 12H shows growth curves of tumors in immunodeficient mice (n=10 for days 12, 17, and 21; n=7 for day 25); FIG. 12I shows representative bioluminescent images of mice in FIG. 12H following treatment with vehicle or F5446 at day 25; FIG. 12J shows images of mammary tumors dissected from mice in FIG. 12H following treatment with vehicle or F5446 at day 25; FIG. 12K shows representative IHC images of tumors stained with anti-γH2AX or anti-cleaved Caspase 3 antibody (top panel) and quantification (n=6) (bottom panel). Scale bar, 50 μm; FIG. 12L shows Real-Time Quantitative Reverse Transcription PCR (qRT-PCR) analysis for tumors dissected from mice in FIG. 12H (n=3); FIG. 12M shows growth curves of tumors in syngeneic immunocompetent mice (n=6); FIG. 12N shows representative bioluminescent images of tumors in FIG. 12M at day 28 (left panel) and quantification of total body radiance (n=6) (right panel); FIG. 12O shows survival curves for mice in FIG. 12M. Data represent mean±SEM. ns, not significant; *p<0.05; **p<0.01; ***p<0.001; ****p<0.0001 by one-way ANOVA followed by Dunnett's multiple comparisons test in FIGS. 12B, 12C, 12D (for CD45+ cells), and FIG. 12E, Tukey's multiple comparisons test in FIG. 12N, two-way ANOVA followed by Tukey's multiple comparisons test in FIGS. 12A, 12D (for CD8+T and NK cells), FIGS. 12F, 12H, 12L, and 12M, Sidak's multiple comparisons test in FIG. 12K, and log-rank test in FIGS. 12G and 12O.

FIGS. 13A-13J show that FBXO44 is associated with poor clinical outcomes in cancer patient datasets: FIG. 13A shows analysis of FBXO44 expression in indicated cancer types relative to normal adjacent tissue (Oncomine, Compendia Bioscience, Ann Arbor, MI); FIG. 13B shows representative IHC images (left panels) and quantification of FBXO44 expression in normal breast tissue and breast tumors (n=8 normal, n=24 stage II, and n=16 stage III) (right panel). Scale bar, 0.5 mm; inset scale bar, 50 μm; FIG. 13C shows survival plots for patients with FBXO44 high- vs. low-expressing tumors (www.kmplot.com); FIG. 13D shows Pan-cancer analysis of TCGA dataset for FBXO44 expression with indicated gene expression signatures; FIG. 13E shows pathway enrichment map for GSEA for gene sets enriched among significantly upregulated (red) or downregulated (blue) genes in FBXO44 high-vs. low-expressing tumors in pan-cancer analysis of the TCGA dataset. FDR<0.1; FIG. 13F shows GSEA analysis of various immune-stimulatory pathways in FBXO44 high- vs. low-expressing tumors in pan-cancer analysis of the TCGA dataset; FIG. 13G shows correlation analysis between FBXO44 expression level and z-scores of the indicated gene sets in different cancer types from the TCGA dataset; FIG. 13H shows boxplots of FBXO44-immune gene signature z-scores in non-responder and responder groups of patients with anti-PD-1 or TIL therapy in the indicated datasets; FIG. 13I shows heatmap of the FBXO44-immune gene signature differentially enriched in responder versus non-responder patients in Harel anti-PD-1 therapy dataset; FIG. 13J shows model of FBXO44/SUV39H1 inhibition-induced antitumor effects and enhancement of immunotherapy response. Data represent mean±SEM. *p<0.05; ****p<0.0001 by two-way ANOVA followed by Tukey's multiple comparisons test in FIG. 13B, and unpaired Student's t-test in FIG. 13H.

FIGS. 14A-14E show that expression of FBXO44 is inversely correlated with clinical outcomes in cancer patient datasets: FIG. 14A shows scatter plots of the expression level correlation between FBXO44 and the indicated representative genes from immune signatures in FIG. 13D. The best-fit linear lines, Spearman's p, and p values are shown; FIG. 14B shows GSEA analysis of the indicated immune-stimulatory pathways in FBXO44 high- vs. low-expressing tumors in pan-cancer analysis of the TCGA dataset. NES and p values are shown; FIG. 14C shows correlation analysis between FBXO44 expression level and z-scores of gene sets for activation of ATR in response to replication stress in different cancer types from the TCGA dataset. p values from Spearman correlation were corrected for multiple testing as FDR; FIG. 14D shows generation of FBXO44-immune gene signature (83 immune-related genes) based on GSEA analysis for FBXO44 KD RNA-seq data; FIG. 14E shows heatmap of the FBXO44-immune gene signature differentially enriched in responder versus non-responder patients in Harel TIL therapy dataset.

### DETAILED DESCRIPTION

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity and can includes any human or non-human animal. The term “non-human animals” includes vertebrates such as non-human primates, but is not limited to sheep, dogs, cats, rabbits and ferrets, rodents such as mice, rats and guinea pigs, bird species such as chicken, amphibians, and reptiles. In a preferred embodiment, the subject is a mammal, such as a human, In other embodiments, the term subject can include non-human primate, sheep, dog, cat, rabbit, ferret or rodent. The terms “subject”, “patient” and “individual” are used interchangeably herein. The subject can be tissues, cells and their progeny of a biological entity obtained in vivo or cultured in vitro. The subject can be a mammal. The subject may be diagnosed or suspected of being at high risk for a disease. In some cases, the subject is not necessarily diagnosed or suspected of being at high risk for the disease.

The term “in vivo” is used to describe an event that takes place in a subject's body. The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made. A therapeutically effective amount of a drug or a chemical entity includes a “prophylactically effective amount”, which is administered alone to a subject at risk of developing cancer (e.g., a subject in a pre-malignant condition) or to a subject at risk of suffering from a recurrence of cancer, or when administered in combination with another biological agent, it is any amount of a drug that inhibits the occurrence or recurrence of cancer. In a preferred embodiment, the prophylactically effective amount completely prevents the occurrence or recurrence of the cancer. “Inhibiting” the occurrence or recurrence of cancer means reducing the likelihood of occurrence or recurrence of cancer, or entirely preventing the occurrence or recurrence of cancer.

“Administering” refers to physically introducing a composition comprising a therapeutic agent to a particular subject using any of a variety of methods and delivery systems known to those of skill in the art. Preferred routes of administration for the therapeutic agents of the present invention include intravenous, intramuscular, subcutaneous, intraperitoneal, spinal or other parenteral routes of administration, for example by injection or infusion. As used herein, “parenteral administration” refers to a mode of administration other than intestinal and topical administration, usually by injection, and intravenous, intramuscular, intraarterial, intramenal, intralymphatic, intralesional, Intra-capsular, intraorbital, intracardiac, intradermal, intraperitoneal, transtraminal, subcutaneous, subcutaneous, intraarticular, subarticular, subarachnoid, intrathecal, dural and intrasternal injections and injections, as well as in vivo electroporation, is not limited thereto. On the other hand, the therapeutic agents of the present invention can be administered through a non-parenteral route, such as topical, epidermal or mucosal route of administration, for example intranasal, oral, vaginal, rectal, sublingual or topical administration. Administration can also be carried out, for example, once, multiple times, and/or over one or more extended periods of time.

By “agent” is meant any small molecule chemical compound, antibody, nucleic acid molecule, or polypeptide, or fragments thereof. The term “immunotherapeutic agent” refers in general to any agent which produces a therapeutic effect by targeting the immune system or a component thereof. As used herein, the immunotherapeutic agent typically promotes an immune response, e.g., the agent may be an immunostimulatory agent or an inhibitor of an immunosuppressive agent (i.e., an anti-immunosuppressive agent). The term can include immunogenic compositions and vaccines, e.g., neoplasia vaccines comprising neoantigenic peptides. Immunotherapeutic agents can also include checkpoint blockers or inhibitors, chimeric antigen receptors (CARs), and adoptive T-cell therapy.

Immune checkpoints are inhibitory pathways that slow down or stop immune reactions and prevent excessive tissue damage from uncontrolled activity of immune cells. By “checkpoint inhibitor” or “immune checkpoint inhibitor” is meant to refer to any small molecule chemical compound, antibody, nucleic acid molecule, or polypeptide, or fragment thereof, that inhibits the inhibitory pathways, allowing more extensive immune activity. A “checkpoint inhibitor” or “immune checkpoint inhibitor” can also be an agent that stimulates a preexisting immune response. In certain embodiments, the checkpoint inhibitor is an inhibitor of the programmed death-1 (PD-1) pathway. In some cases, the checkpoint inhibitor is a chemical entity that blocks PD-1, PD-L1, B7-1, B7-2, or CTLA-4. In some cases, the inhibitor is an antibody. In additional embodiments, the checkpoint inhibitor is targeted at another member of the CD28 CTLA4 Ig superfamily such as BTLA, LAG3, TIME3, VISTA, TIGIT, ICOS, PDL1 or KIR (Page et al., Annual Review of Medicine 65:27 (2014)). In further additional embodiments, the checkpoint inhibitor is targeted at a member of the TNFR superfamily such as CD40, OX40, CD137, GITR, CD27 or TIM-3. In some cases, targeting a checkpoint inhibitor is accomplished with an inhibitory antibody or similar molecule. In other cases, it is accomplished with an agonist for the target; examples of this class include the stimulatory targets OX40 and GITR.

The term “combination” embraces the administration of therapeutic agents (or example, a FBX044 inhibitor) and an immunogenic composition (e.g., a pooled sample of neoplasia/tumor specific neoantigens, or one or more checkpoint inhibitors), as part of a treatment regimen intended to provide a beneficial (additive or synergistic) effect from the co-action of one or more of these therapeutic agents. The combination may also include one or more additional agents, for example, but not limited to, chemotherapeutic agents, anti-angiogenesis agents and agents that reduce immune-suppression. The beneficial effect of the combination includes, but is not limited to, pharmacokinetic or pharmacodynamic co-action resulting from the combination of therapeutic agents. Administration of these therapeutic agents in combination typically is carried out over a defined time period (for example, minutes, hours, days, or weeks depending upon the combination selected).

“Combination therapy” is intended to embrace administration of the therapeutic agents disclosed herein in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. For example, one combination of the present invention may comprise a FBX044 inhibitor (for example, an FBX044 siRNA molecule) and a checkpoint inhibitor administered at the same or different times, or they can be formulated as a single, co-formulated pharmaceutical composition comprising the two compounds. As another example, a combination of the present invention (e.g., a FBX044 siRNA molecule and a pooled sample of tumor specific neoantigens and a checkpoint inhibitor and/or an anti-CTLA4 antibody) may be formulated as separate pharmaceutical compositions that can be administered at the same or different time. As used herein, the term “simultaneously” is meant to refer to administration of one or more agents at the same time. For example, in certain embodiments, a neoplasia vaccine or immunogenic composition and a checkpoint inhibitor are administered simultaneously. Simultaneously includes administration contemporaneously, that is during the same period of time. In certain embodiments, the one or more agents are administered simultaneously in the same hour, or simultaneously in the same day. Sequential or substantially simultaneous administration of each therapeutic agent can be affected by any appropriate route including, but not limited to, oral routes, intravenous routes, sub-cutaneous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, one component of a particular combination may be administered by intravenous injection while the other component(s) of the combination may be administered orally. The components may be administered in any therapeutically effective sequence. The phrase “combination” embraces groups of compounds or non-drug therapies useful as part of a combination therapy.

The terms “cancer”, “malignant tumor”, “neoplasm”, ‘tumor”, and “carcinoma” are used interchangeably herein and refer to relatively abnormal, uncontrolled, and refers to a cell that exhibits an aberrant growth phenotype characterized by a significant loss of cell proliferation control. In general, cells of interest for detection and treatment in the present application include pre-cancerous (e.g., benign), malignant, metastatic, metastatic and non-metastatic cells. The teachings of the present disclosure may relate to any and all cancers. By “neoplasia” is meant any disease that is caused by or results in inappropriately high levels of cell division, inappropriately low levels of apoptosis, or both. For example, “cancer” is an example of a neoplasia. Examples of cancers include, without limitation, leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

The term “cancer vaccine” is meant to refer to a pooled sample of cancer/tumor specific neoantigens, for example at least two, at least three, at least four, at least five, or more neoantigenic peptides. A “vaccine” is to be understood as meaning a composition for generating immunity for the prophylaxis and/or treatment of diseases (e.g., neoplasia/tumor). Accordingly, vaccines are medicaments which comprise antigens and are intended to be used in humans or animals for generating specific defense and protective substance by vaccination. A “cancer vaccine composition” can include a pharmaceutically acceptable excipient, carrier or diluent.

The term “pharmaceutically acceptable” refers to approved or approvable by a regulatory agency of the Federal or a state government or listed in the U.S. Pharmacopeia or other generally recognized pharmacopeia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

In embodiments of the present invention, cytolytic activity of a tumor is monitored in order to determine susceptibility of the tumor to immunotherapy. In some cases, mutations in tumor cells may lead to tumor resistance to cytolytic activity. In such cases, despite a high level of cytotoxic activity in the tumor, tumor cells may evade immune system attack by mutations involved in, for example, cytolytic cell death and/or antigen presentation. In embodiments of the present invention, tumor resistance to cytolytic activity may be overcome by targeting an immunotherapy at subjects whose tumors are vulnerable to immune system attack, but which have avoided destruction by cytotoxic T cells and/or natural killer cells through immunosuppressive mutations. By boosting the immune response in such subjects, tumor evasion of cytolytic activity can be overcome leading to an enhanced therapeutic effect. In a similar manner, in further embodiments of the invention where tumor mutations result in suppression of cytolytic activity, immunotherapy may be used to overcome such suppression and lead to cytolytic destruction of tumor cells. Thus, in one embodiment, a tumor is assessed for its ability to mount an immunological response or tumor immunity.

In another embodiment, the invention provides a method for selecting patients most likely to benefit from an immunotherapy, e.g., a combination therapy as described therein. In one embodiment patients are selected based on the notion that effective natural anti-tumor immunity requires a cytolytic immune response. In another embodiment patients are selected based on the need for additional activation of the immune system. In another embodiment patients are selected based on the need for de-repression of the immune system. In another embodiment patients are selected based on the ability to present tumor neoantigens to the immune system. In one embodiment patients are selected based on the ability to produce a cytolytic response and the ability to present tumor neoantigens to the immune system. In another embodiment patients are selected based on the ability to produce a cytolytic response, the ability to present tumor neoantigens to the immune system, and the need for additional immune stimulation. In another embodiment patients are selected that have tumors with mutations encoding neoantigens. In one embodiment patients are selected that have cytolytic activity, neoantigens expressed in their tumors, and that do not have mutations that prevent the presentation of antigens or prevent cytolytic killing by T-cells.

“Immune response” refers to cells of the immune system (eg, T lymphocytes, B lymphocytes, natural killer (NK) cells, macrophages, eosinophils, mast cells, dendritic cells and neutrophils), and invading pathogens, cells or tissues infected with pathogens.

“Immunotherapy agent” or a “Cancer immunotherapy agent” refers to a substance, signaling pathway or component thereof that modulates the immune response. “Modulating”, “modifying” or “modulating” an immune response refers to a cell of the immune system or any alteration in the activity of such cells. Such modulation includes stimulation or inhibition of the immune system, which may be evident by an increase or decrease in the number of various cell types, an increase or decrease in the activity of these cells, or any other change that may occur within the immune system. Both inhibitory and stimulating immunomodulatory factors have been identified, some of which may exhibit enhanced function in the cancer microenvironment. In some cases, an immunotherapy agent can increase the effectiveness or potency of an existing immune response in a subject. Such an increase in effectiveness and potency can be achieved, for example, by overcoming mechanisms that inhibit the endogenous host immune response or by stimulating the mechanisms that enhance the endogenous host immune response.

The term “immunotherapy” refers to a method comprising stimulating, eliciting, increasing, enhancing, sustaining, and/or improving the stimulation of new immune response or of a preexisting immune response. Thus, “stimulating an immune response” as an immunotherapy refers to enhancing the therapeutic efficacy, increasing survival time, slowing the progression of a cancerous tumor or shrinking the cancerous tumor size, preventing the spread of a tumor or of metastases, preventing or slowing the recurrence of treated cancer, eliminating cancer cells not killed by earlier treatments, targeting potential cancer cells or targeting antigens derived from a virus associated with cancer.

In the methods described herein, the immunotherapeutic agent (for example, an PD-1 or PD-L1 inhibitor) are administered in combination with a chemical entity capable of inhibiting FBX044 are administered in an amount effective to stimulate an immune response in the subject individual at a dose sufficient to generate an effective immune response without unacceptable toxicity. As will be understood by one of skill in the art, the magnitude of the immune response and the maintenance of that response may have varying degrees which will be recognized a having a potential therapeutic or prophylactic benefit.

A “inhibitor” of signaling refers to a compound or agent that antagonizes or decreases the initiation, reception or transmission of a signal, which is signal stimulating or inhibitory by any component of the signaling pathway, such as a receptor or its ligand.

The recitation of a listing of chemical groups in any definition of a variable herein includes definitions of that variable as any single group or a combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The section headings used herein are for organizational purposes only and are not to be construed as limiting the subject matter described.

### FBX044 Inhibitors

In some cases, the methods described herein comprise administering to a subject suffering from cancer a composition comprising a therapeutically effective amount of a FBX044 inhibitor as indicated in Shen and Spruck, “Targeting FBXO44/SUV39H1 elicits tumor cell-specific DNA replication stress and viral mimicry,” Cell Stress 5(3):37-39 (2021) or Shen et. al, “FBXO44 promotes DNA replication-coupled repetitive element silencing in cancer cells,” Cell 184(2):352-369 (2021) (both incorporated by reference herein in their entireties). The term “FBX044” or “FBXO44” as used herein refers to human FBX044, variants of human FBX044, isoforms and species homologs, and analogs having at least one common epitope with FBX044.

In some cases, the FBX044 inhibitor can be a nucleic acid-based inhibitor. In some cases, the nucleic acid can be DNA. In some cases, the FBX044 inhibitor is an RNA-based inhibitor. In some embodiments, the FBX044 inhibitor can be an siRNA. In some cases, the FBX044 inhibitor can be a peptide, for example, an antibody. In other embodiments, the FBX044 inhibitor can be a small molecule. In some cases, the FBX044 inhibitor can comprise a nucleic acid sequence indicated in Shen et. al., “FBXO44 promotes DNA replication-coupled repetitive element silencing in cancer cells,” Cell 184(2):352-369 (2021) (incorporated by reference herein in its entirety).

In some cases, the methods described herein comprise administering an inhibitor of FBX044 to a subject in need thereof. In some cases, in the methods described herein, FBX044 inhibitors are administered in an amount effective to stimulate an immune response resulting in H3K9me3-mediated transcriptional silencing of REs in cancer cells. In some cases, in the methods described herein, FBX044 inhibitors are administered in an amount effective to stimulation of immune response in the subject. In some cases, in the methods described herein, FBX044 inhibitors are administered in an amount effective to stimulate an immune response resulting in stimulated antiviral pathways in the subject. In some cases, in the methods described herein, FBX044 inhibitors are administered in an amount effective to stimulate an immune response resulting in increase Interferon signaling in the subject in need thereof. In some cases, in the methods described herein, FBX044 inhibitors are administered in an amount effective to stimulate an immune response resulting in increased replication stress in the subject as compared to the immune response in the subject prior to administration of the FBX044 inhibitors.

In some cases, the administration of the FBX044 inhibitor resulting in expression of endogenous retroelements can promote accumulation of double-stranded (ds)RNA and dsDNA replication intermediates in the cytoplasm of cells, where they are recognized by pattern recognition receptors such as RIG-I/MDA5 and cGAS that activate the adaptor proteins MAVS and STING, respectively. In some cases, the administration of the FBX044/SUV39H1 inhibition in cancer cells can transcriptionally activate various RE subtypes, including satellite repeats, ERVs, and LINE-1, resulting in the accumulation of cytosolic dsRNA and dsDNA replication intermediates and activation of RIG-I/MDA5-MAVS and cGAS-STING antiviral pathways, respectively. In some cases, FBXO44 inhibition can promote DSBs and genomic instability, as indicated by the presence of cGAS+H2AX+ micronuclei, resulting in activated IFN signaling, enhanced antigen presentation, and stimulated various cytokines and ligands that function to recruit cytotoxic T and natural killer (NK) cells, thereby enhancing tumor cell immunogenicity. In some cases, inhibition of FBX044 by genetic knockdown or pharmacologic inhibition of SUV39H1 can result in restriction of the proliferation of breast, lung, colon, and glioblastoma cancer cells in vitro and tumor growth in immunodeficient mice. Given that FBX044/SUV39H1 inhibition enhanced cancer cell immunogenicity, we next examined if it could modulate antitumor immune response using a preclinical mouse breast cancer model. In some cases, FBX044/SUV39H1 inhibition can stimulate the intratumoral infiltration of cytotoxic CD8+T and NK cells. In addition, in the methods herein, SUV39H1 inhibition can synergize with anti-PD-1 immunotherapy to prevent tumor growth and increase survival of the subject. In some cases, administration of FBX044 inhibitors can cause viral mimicry selectively in cancer cells, leading to inhibition of tumor growth and enhanced anti-PD-1 therapy response in a subject.

### Checkpoint Inhibitors

The “programmed death-1 (PD-1)” receptor refers to an immunosuppressive receptor belonging to the CD28 family. PD-1 is primarily expressed on T cells that have already been activated in vivo and binds to two ligands, PD-L1 and PD-L2. The term “PD-1” as used herein refers to human PD-1 (hPD-1), variants of hPD-1, isoforms and species homologs, and analogs having at least one common epitope with hPD-1. Include. The complete hPD-1 sequence can be found under Genbank accession number U64863.

“Programmed death ligand-1 (PD-L1)” is two cell surface glycoprotein ligands for PD-1 (the other is PD-), which downregulates cytokine secretion and T cell activation upon binding to PD-1. L2) is one of. The term “PD-L1” as used herein refers to human PD-L1 (hPD-L1), variants of hPD-L1, isotypes and species homologs, and analogs having at least one consensus epitope with hPD-L1. Include. The complete hPD-L1 sequence can be found under Genbank accession number Q9NZQ7.

In some embodiments, the present disclosure comprises administering to a subject suffering from cancer a composition comprising a therapeutically effective amount of an PD-4,1 inhibitor of the present invention or an antigen-binding portion thereof, in combination with an FBX044 inhibitor. In a preferred embodiment, the subject is a human. in certain embodiments, the inhibitor is the Ab or antigen-binding portion thereof is an IgG1 or IgG4 isotype. In other embodiments, the Ab or antigen-binding portion thereof is a mAb or antigen-binding portion thereof. The anti-PD-1 Ab used in the present method may be any therapeutic anti-PD-1 Ab of the present invention. In a preferred embodiment, the anti-PD-1 Ab is a mAb, which may be a chimeric, humanized or human Ab. In certain embodiments, the anti-PD-1 Ab is a CDR1 in the heavy chain variable region of 17D8, 2D3, 4H1, 5C4 (nivolumab), 4A11, 7D3 or 5F4, as described and clearly defined in U.S. Pat. No. 8,008,449, It includes the CDR2 and CDR3 domains and includes the CDR1, CDR2 and CDR3 domains within the light chain variable region. In a further embodiment, the anti-PD-1 Ab comprises heavy and light chain variable regions of 17D8, 2D3, 4H1, 5C4 (nivolumab), 4A11, 7D3 or 5F4, respectively. In a further embodiment, the anti-PD-1 Ab is 17D8, 2D3, 4H1, 5C4 (nivolumab), 4A11, 7D3 or 5F4. In a preferred embodiment, the anti-PD-1 Ab is nivolumab. PD-1 antibody Antibodies to PD-1 are U.S. Pat. Nos. 8,735,553, 8,617,546, 8,008,449, 8,741,295, and 8,552,154, 8,354,509, 8,779,105, 7,563,869, 8,287,856, 8,927,697 8,088,905, 7,595,048, 8,168,179, 6,808,710, 7,943,743, 8,246,955, and 8,217,149. It is envisioned that any known antibody can be used in this method. In some embodiments, a mouse monoclonal antitarget antibody for human PD-1 is used in the method. For example, In vivo MAb antih PD-1 (BioXCell, Clone: RMP1-14, catalog number BE0146). Anti-PD-1 antibodies have been shown to be effective in human therapy, e.g., pembrolizumab (KEYTRUDA®, Merck Sharp & Dohme Corp.), which is an anti-PD-1 antibody approved for use in human therapy). In some cases, the anti-PD-1 antibody may be cemiplimab. In some cases, the anti-PD-1 antibody may be nibolumab (Opdivo®, Bristor-Myers Squibb). Additional PD-1 antibodies, such as Pidirisumab (CT-011) (CureTech Ltd.), are under clinical development. In some cases, the anti-PD-L1 antibody may be selected from atezolizumab, durvalumab, or avelumab.

In some embodiments, the present disclosure comprises administering to a subject suffering from cancer a composition comprising a therapeutically effective amount of an CTLA4 inhibitor of the present invention or an antigen-binding portion thereof, in combination with an FBX044 inhibitor. Anti-CTLA-4 antibodies of the present invention bind human CTLA-4 to interfere with the interaction between CTLA-4 and human B7 receptors. Since the interaction between CTLA-4 and B7 transforms a signal leading to the inactivation of T-cells carrying the CTLA-4 receptor, interfering with the interaction effectively induces the activation of these T cells. Anti-CTLA-4 Abs are described, for example, in U.S. Pat. Nos. 6,051,227, 7,034,121, PCT application publications WO 00/37504 and WO 01/14424, each of which is incorporated by reference in its entirety. An exemplary clinical anti-CTLA-4 Ab is human mAb 10D1 (now known as ipilimumab and marketed as YERVOY®) as described in U.S. Pat. No. 6,984,720, which is incorporated by reference in its entirety. In certain aspects of any of the methods, the anti-CTLA-4 Ab is a mAb. In certain other embodiments, the anti-CTLA-4 antibody is a chimeric, humanized or human antibody. In an embodiment, the anti-CTLA-4 antibody is ipilimumab.

In some cases, in the methods described herein, the FBX044 inhibitor may be administered with an inhibitor that in combination blocks, FBX044, PDL-1 and/or CTLA4. Combination blockade of FBX044, PD-1 and CTLA-4 can also be further combined with standard cancer treatments. For example, combination blockade of PD-1 and CTLA-4 can be effectively combined with chemotherapeutic therapy, for example with dacarbazine or IL-2 to treat MEL. In these cases, it may be possible to reduce the dose of the chemotherapeutic agent. The scientific rationale behind the combination of PD-1 and CTLA-4 blockade with chemotherapy is that cell death, a result of the cytotoxic action of most chemotherapy compounds, results in increased levels of tumor antigens in the tumor presentation pathway. It should be done. Other combination therapies that may result in combination blocking and synergistic effects of PD-1 and CTLA-4 through cell death include radiation, surgery, or hormone deprivation. Each of these protocols creates a source of tumor antigens in the host. Angiogenesis inhibitors can also be combined with a combination blockade of PD-1 and CTLA-4. Inhibiting angiogenesis kills tumor cells, which may also be a source of tumor antigens to be supplied into the host antigen presentation pathway.

In some cases, in the methods described herein, the FBX044 inhibitor may be administered with an inhibitor directed to other immune check point molecules. In some cases, the inhibitors may be directed to other immune check points, for example, lymphocyte activation gene 3 LAG 3, T-cell membrane protein 3, and V-domain immunoglobulin suppressor of T-cell activation (VISTA). In some cases, the inhibitors may be directed against TIGIT signal transduction pathway. In some cases, the inhibitors may be directed against the ligands for TIGIT signaling. In some cases, the inhibitor may be directed against BTLA signaling.

In some cases, the chemical entity that is an inhibitor of an immune checkpoint may be a non-antibody type immune checkpoint inhibitor.

### Cytokine Therapy Agents

In some cases, the FBX044 inhibitor may be administered in combination with cytokine therapy agents. In some embodiments, cytokine therapy agents that regulate the immune system, are recognizable according to their structures.

In some cases, in the methods described herein, the FBX044 inhibitor may be administered with a cytokine therapy agent of the interleukin family. The interleukin family includes cytokines such as IL-2, 3, 4, 5, 6, 7, 9, 12, 13, and 15. These cytokines are small (10-20 kDaltons) proteins that all share a 3-dimensional structure of 4 antiparallel alpha helices. The receptors of this cytokine family share amino acid sequence homologies especially in their extracellular domains.

In some cases, in the methods described herein, the FBX044 inhibitor may be administered with a cytokine therapy agent of the tumor necrosis factor (TNF) family. The tumor necrosis factor (TNF) family includes compounds such as TNF-α, TNF-β (lymphotoxin), nerve growth factor (NGF), and the CD40, Fas, CD27, and CD30 ligands. The ligands of this family are either secreted or remain membrane anchored, and function as homotrimers of about 15 kDalton monomers. The receptors of this family share amino acid sequence homologies.

In some cases, in the methods described herein, the FBX044 inhibitor may be administered with a cytokine therapy agent of the interferon (IFN) family. The IFN family includes compounds such as IFN-α, IFN-β, and IFN-γ, and is distinguished by the unique biologic property of stimulating cells to prevent viral replication.

In some cases, in the methods described herein, the FBX044 inhibitor may be administered with a cytokine therapy agent of the interferon chemokine family. The chemokine family includes molecules such as IL-8, macrophage inhibiting protein (MIP), and Rantes. These cytokines are small (about 10 kDaltons) and bind to a distinct family of receptors that have 7 membrane spanning alpha helices, and that are coupled to guanine nucleotide binding proteins (G proteins).

Administration of FBX044 with Cancer Vaccines

In some cases, in the methods described herein, the FBX044 inhibitor may be administered with a immunopotentiator such as cancer vaccine. Cancer vaccines can include preventive or prophylactic vaccines (intended to prevent the development of cancer in healthy people), and therapeutic vaccines (intended to treat existing cancers by enhancing the body's natural defenses against cancer).

In some cases, in the methods described herein, the FBX044 inhibitor may be administered with a cancer vaccine that may be an RNA vaccine. In some cases, the RNA vaccines comprise a ribonucleic acid (RNA) polynucleotide having an open reading frame encoding at least one cancer antigen polypeptide or immunogenic fragment thereof (eg, an immunogenic fragment capable of inducing an immune response against cancer). Other embodiments include at least one ribonucleic acid (RNA) polynucleotide having an open reading frame encoding two or more antigens or epitopes capable of inducing an immune response against cancer. In some embodiments, the mRNA cancer vaccine has an open reading frame encoding a concatemer of at least two activated oncogene variant peptides. In some embodiments, the mRNA cancer vaccine further comprises an mRNA having an open reading frame encoding one or more conventional cancer antigens.

In some cases, in the methods described herein, the FBX044 inhibitor may be administered with a cancer vaccine that is a peptide cancer vaccine. In some embodiments, the peptide cancer vaccine encodes a concatemer of at least two activated oncogene variant peptides. In some cases, the cancer peptide vaccines comprise a polypeptide encoding at least one cancer antigen polypeptide or immunogenic fragment thereof (e.g., an immunogenic fragment capable of inducing an immune response against cancer).

Combination with Other Therapies

In some instance, the administration of the therapeutic agents disclosed herein can be used in combination with conventional cancer therapies or pharmaceutical formulations useful for treating cancer or infectious diseases. These treatments can include surgical procedures, radiation therapy and/or ablation therapy (e.g., laser therapy, infrared therapy and the like).

Cancer therapies including dendritic cell therapy, chemokines, cytokines, tumor necrosis factors (e.g., TNF-α), chemotherapeutic agents (e.g., adenosine analogs (e.g., cladribine, pentostatin), alkyl sulfanates (e.g., busulfan)), anti-tumoral antibiotics (e.g., bleomycin, dactinomycin, daunorubicin, doxorubicin, epirubicin, idarubicin, mitoxantrone, mitomycin), aziridines (e.g., thiotepa), camptothecin analogs (e.g., irinotecan, topotecan), cryptophycins (e.g., cryptophycin 52, cryptophicin 1), dolastatins (e.g., dolastatin 10, dolastatin 15), enedyine anticancer drugs (e.g., esperamicin, calicheamicin, dynemicin, neocarzinostatin, neocarzinostatin chromophore, kedarcidin, kedarcidin chromophore, C-1027 chromophore, and the like), epipodophyllotoxins (e.g., etoposide, teniposide), folate analogs (e.g., methotrexate), maytansinoids (e.g., maytansinol and maytansinol analogues), microtubule agents (e.g., docetaxel, paclitaxel, vinblastine, vincristine, vinorelbine), nitrogen mustards (e.g., chlorambucil, cyclophosphamide, estramustine, ifosfamide, mechlorethamine, melphalan), nitrosoureas (e.g., carmustine, lamustine, streptoxacin), nonclassic alkylators (e.g., altretamine, dacarbazine, procarbazine, temozolamide), platinum complexes (e.g., carboplatin, cisplatin), purine analogs (e.g., fludarabine, mercaptopurine, thioguanine), pyrimidine analogs (e.g., capecitabine, cytarabine, depocyt, floxuridine, fluorouracil, gemcitabine), substituted ureas (e.g., hydroxyurea)]; anti-angiogenic agents (e.g., canstatin, troponin I), biologic agents (e.g., ZD 1839, virulizin and interferon), antibodies and fragments thereof (e.g., anti EGFR, anti-HER-2/neu, anti-KDR, IMC-C225), anti-emetics (e.g., lorazepam, metroclopramide, and domperidone), epithelial growth factor inhibitors (e.g., transforming growth factor beta 1), anti-mucositic agents (e.g., dyclonine, lignocaine, azelastine, glutamine, corticoid steroids and allopurinol), anti-osteoclastic agents (e.g., bisphosphonates (e.g., etidronate, pamidronate, ibandronate, and osteoprotegerin)), hormone regulating agents (e.g., anti-androgens, LHRH agonists, anastrozole, tamoxifen), hematopoietic growth factors, anti-toxicity agents (e.g., amifostine), kinase inhibitors (gefitinib, imatinib), and mixtures of two or more thereof.

With regard to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype” or “drug response genotype”). Thus, methods for tailoring an individual's prophylactic or therapeutic treatment according to that individual's drug response genotype may be used. Pharmacogenomics allows a clinician or physician to target prophylactic or therapeutic treatments to patients who will most benefit from the treatment and to avoid treatment of patients who will experience toxic drug-related side effects. The clinician or physician can thereby tailor the type of treatment that may be necessary to the specific patient.

In the described methods of immunotherapy, the immune response may be further augmented by the administration of compounds that may act as an immunostimulatory compound. Exemplary immunostimulatory compounds include toll like receptor (TLR) agonists (e.g., TLR4, TLR7, TLR9), N-acetylmuramyl-L-alanine-D-isoglutamine (MDP), lipopolysaccharides (LPS), genetically modified and/or degraded LPS, alum, glucan, colony stimulating factors (e.g., EPO, GM-CSF, G-CSF, M-CSF, pegylated G-CSF, SCF, IL-3, IL6, PIXY 321), interferons (e.g., γ-interferon, α-interferon), interleukins (e.g., IL-2, IL-7, IL-12, IL-15, IL-18), MEW Class II binding peptides, saponins (e.g., QS21), unmethylated CpG sequences, 1-methyl tryptophan, arginase inhibitors, cyclophosphamide, antibodies that block immunosuppressive functions (e.g., anti-CTLA4 antibodies), and mixtures of two or more thereof. Exemplary TLR4 agonists include lipopolysaccharides (LPS); E. coli LPS; and P. gingivalis LPS. Exemplary TLR7 agonists include imidazoquinoline compounds (e.g., imiquimod, resiquimod and the like); and loxoribine. Other exemplary immunostimulatory compounds may include ipilimumab (YERVOY®, Bristor-Myers Squibb Company), anti-VEGF-A, or a combination of cytokines, growth factors, other inhibitors and antibodies against other target antigens, such as CTLA-4. Bevacizumab (AVASTIN®, Genentech), an antibody against EGFR, erlotinib (TARCEVA®, Genentech and OSI Pharmaceuticals), oral Bcr-Abl tyrosone kinase (tyrosone), dasatinib (SPRYCEL®, Bristol-Myers Squibb Company), IL-21, pegged IFN-α2b, the tyrosine kinase inhibitor axitinib (INLYTA®, Phizer, Inc.), and. Other therapeutic agents such as the MEK inhibitor tramethinib (MEKINIST®, GlaxoSmithKline) can also be used in combination with the methods and compositions disclosed herein.

### Pharmaceutical Compositions

Compositions comprising the chemical entities of the disclosure herein may comprise a pharmaceutical composition, for example a pharmaceutical composition containing one Ab or combination of Abs, or a nucleic acid capable of binding to an antigen (e.g., siRNA against FBX044), and a pharmaceutically acceptable carrier. As used herein, “pharmaceutically acceptable carrier” includes any and all solvents, dispersion media, coatings, antibacterial and antifungal agents, isotonic and absorption delaying agents, and other agents that are physiologically compatible. Preferably, the carrier is suitable for intravenous, intramuscular, subcutaneous, parenteral, spinal or epidermal administration (e.g., by injection or infusion). Pharmaceutical compositions of the present invention may comprise one or more pharmaceutically acceptable salts, antioxidants, aqueous and non-aqueous carriers, and/or adjuvants such as preservatives, wetting agents, emulsifying agents and dispersing agents. In addition, the compound may form a solvate with water or a common organic solvent. Such solvates are also envisioned.

The pharmaceutical compositions of the present disclosure comprising the inhibitors described herein as active ingredients may comprise a pharmaceutically acceptable carrier or additive, depending on the route of administration. Examples of such carriers or additives include water, pharmaceutically acceptable organic solvents, collagen, polyvinyl alcohol, polyvinylpyrrolidone, carboxyvinyl polymers, sodium carboxymethyl cellulose, sodium polyacrylate, sodium alginate, water-soluble dextran, carboxy. Methylstarch sodium, pectin, methylcellulose, ethylcellulose, xanthan gum, arabic gum, casein, gelatin, agar, diglycerin, glycerin, propylene glycol, polyethylene glycol, vaseline, paraffin, stearyl alcohol, stearic acid, human serum albumin (HSA), mannitol, Sorbitol, lactose, pharmaceutically acceptable surfactants and the like. The additives used are selected from, but not limited to, those described above or combinations thereof, as appropriate, depending on the dosage form of the present disclosure.

The formulation of the pharmaceutical composition varies depending on the route of administration selected (e.g., solution, emulsion). Suitable compositions comprising the chemical entity to be administered, e.g, PD-1 antibody or siRNA against FBX044, can be prepared in a physiologically acceptable vehicle or carrier. With respect to solutions or emulsions, suitable carriers include, for example, aqueous or alcoholic/aqueous solutions, emulsions, or suspensions, including saline and buffering media. Parenteral vehicles may include sodium chloride solution, ringer dextrose, dextrose and sodium chloride, lactated ringer, or fixed oil. Intravenous vehicles can include various additives, preservatives, or fluids, nutrients, or electrolyte supplements.

Various aqueous carriers such as sterile phosphate buffered saline, bacteriostatic water, water, buffered water, 0.4% saline, 0.3% glycine, etc., and albumin, may be subject to mild chemical modification. Other proteins may be included to enhance stability, such as proteins, globulin.

The therapeutic formulation of the chemical entity disclosed herein, for example, a checkpoint inhibitor or a FBX044 inhibitor may be prepared by mixing the inhibitor with the desired degree of purity in the form of a lyophilized formulation or aqueous solution with an optional physiologically acceptable carrier, excipient, or stabilizer (see, for example, Remington's Pharmaceutical Sciences 16th edition, Osol, A. Ed. (1980)), and prepared for storage. Acceptable carriers, excipients, or stabilizers are non-toxic to the recipient at the dosages and concentrations employed, and include saline and/or buffers such as phosphoric acid, citric acid, and other organic acids; antioxidants including ascorbic acid and methionine; preservatives (such as, octadecyldimethylbenzylammonium chloride; hexamethonium chloride; benzalkonium chloride, benzethonium chloride; phenol, butyl or benzyl alcohol; alkylparabens such as methyl or propylparaben; catechol; resorcinol; cyclohexanol; 3-Pentanol; and m-cresol, etc.); low molecular weight (less than about 10 residues) polypeptides; proteins, such as serum albumin, gelatin, or immunoglobulin; hydrophilic polymers such as polyvinylpyrrolidone; amino acids such as glycin, glutamine, asparagine, histidine, arginine, or lysine; monosaccharides, disaccharides, and other carbohydrates including glucose, mannose, or dextrin; chelating agents such as EDTA; sugars such as sucrose, mannitol, trehalose, or sorbitol; salt-forming counterions such as sodium; metal complexes (e.g., Zn-protein complexes); and/or nonionic surfactants such as TWEEN™, PLURONICS™, or polyethylene glycol (PEG).

The formulations herein may also include two or more active compounds (for example, an active chemical entity target PD-1 and an active chemical entity targeting BX044) preferably those having complementary activities that do not adversely affect each other, depending on the needs of the particular indication being treated. Such molecules are preferably present in combination in an amount effective for the intended purpose.

The active ingredient can be in a colloidal drug delivery system (e.g., in a colloidal drug delivery system, for example, in microcapsules prepared by, for example, coacervation techniques or by interfacial polymerization, e.g., hydroxyethyl cellulose or gelatin microcapsules and poly-(methylmethacrylate) microcapsules, respectively. Liposomes, albumin microspheres, microemulsions, nanoparticles, and nanocapsules), or can also be encapsulated in macroemulsions. Such techniques are described in Remington's Pharmaceutical Sciences 16th edition, Osol, A. et al. Ed. (1980).

The formulation used for in vivo administration can be made sterile by filtration through sterile filtration membranes. Aqueous suspensions may contain active compounds mixed with excipients suitable for making aqueous suspensions. Such excipients are suspending agents such as sodium carboxymethyl cellulose, methyl cellulose, hydroxypropyl methyl cellulose, sodium alginate, polyvinylpyrrolidone, tragacant gum and acacia gum. Dispersants or wetting agents may be naturally occurring phospholipids such as, for example. Recitin, or a condensate of an alkylene oxide with a fatty acid, such as polyoxyethylene stearate, or a condensate of an ethylene oxide with a long-chain aliphatic alcohol, such as heptadecaethyl-eneoxycetanol, or polyoxyethylene sorbitol monoole. It can be a condensate of an ethylene oxide such as ate with a partial ester obtained from a fatty acid and hexitol, or a condensate of an ethylene oxide with a partial ester obtained from a fatty acid and hexitol anhydride, such as polyethylene sorbitan monooleate. Aqueous suspensions may also contain one or more preservatives, such as ethyl, or n-propyl, p-hydroxybenzoate.

The chemical entities (e.g., antibodies or inhibitors) described herein can be lyophilized for storage and reconstituted with a suitable carrier prior to use. For example, PD-1 antibodies and FBX044 antibodies described herein can be prepared and administered as a co-formation. In one aspect, at least two of the antibodies recognize and bind to different antigens. In another embodiment, at least two of the antibodies can specifically recognize and bind to different epitopes of the same antigen.

Any suitable lyophilization and reconstruction techniques can be employed. Those skilled in the art will appreciate that lyophilization and reconstitution may result in varying degrees of loss of antibody activity and may have to be supplemented by adjusting the level of use. Dispersible powders and granules suitable for the preparation of aqueous suspensions by the addition of water can result in active compounds mixed with dispersants or wetting agents, suspending agents, and one or more preservatives. Suitable dispersants or wetting agents and suspending agents are exemplified by those already described above.

The concentration of the chemical entity (e.g., PD-1 antibody or FBX044 antibody) in these formulations may vary widely, for example, from less than about 0.5% by weight to about 20% weight, usually from about 1% by weight or at least about 1% by weight to about 15 or 20% by weight. The concentration of the chemical entity is selected based on the liquid volume, viscosity, etc. according to the specific administration mode to be applied. Therefore, a typical pharmaceutical composition for parenteral injection can be configured to contain 1 mL sterile buffered water and 50 mg antibody. A typical composition for intravenous infusion can be configured to include 250 mL of sterile Ringer's solution and 150 mg of antibody. Practical methods for preparing compositions for parenteral administration are known or apparent to those of skill in the art, e.g., Remington's Pharmaceutical Science, 15th ed., Mack Publishing Company, Easton, Pa. The effective dosage of the antibody is in the range of from about 0.01 mg to about 1000 mg per kg body weight per dose.

The pharmaceutical composition can be in the form of a sterile injectable aqueous, oily suspension, dispersion, or sterile powder for an immediate preparation of a sterile injectable solution or dispersion. Suspensions can be formulated according to known techniques using those suitable dispersants or wetting agents and suspending agents described above. The sterile injectable preparation may be a sterile injectable solution or suspension of a non-toxic parenterally acceptable diluent or solvent such as, for example, 1,3-butanediol. The carrier can be a solvent or dispersion medium containing, for example, water, ethanol, polyol (e.g., glycerol, propylene glycol, liquid polyethylene glycol, etc.), a suitable mixture thereof, vegetable oil, Ringer's solution, and isotonic sodium chloride solution. In addition, sterile fixed oils have traditionally been adopted as solvents or suspension media. For this purpose, any non-irritating fixed oil may be employed, including synthetic mono- or diglycerides. In addition, fatty acids such as oleic acid are used in the preparation of injections.

In all cases, the form must be sterile and fluid enough to be easily injected. Appropriate fluidity can be maintained, for example, by the use of coating agents such as lecithin, by the maintenance of the required particle size in the case of dispersion, and by the use of surfactants. It must be stable under manufacturing and storage conditions and must be preserved against the contaminating effects of microorganisms such as bacteria and fungi. Blocking the action of microorganisms can be provided by various antibacterial and antifungal agents such as parabens, chlorobutanol, phenols, sorbic acid, thimerosal and the like. In many cases it is desirable to include isotonic agents such as sugar or sodium chloride. Long-term absorption of the injectable composition can be provided by using an agent that delays absorption in the composition, such as aluminum monostearate or gelatin.

Compositions useful for administration can be formulated with uptake or absorption enhancers to increase their effectiveness. Such enhancers include, for example, salicylate, glycocholate/linoleate, glycolate, aprotinin, bacitracin, SDS, caplate and the like. See, for example, Fix (J. Pharma. Sci., 85: 1282-1285 (1996)) and Oliyai and Stella (Ann. Rev. Pharmacol. Toxicol., 32: 521-544 (1993)).

A composition described herein may be intended for use in inhibiting target activity, including binding of a target to its cognate receptor or ligand, target-mediated signaling, and the like. For example, a composition described herein may be intended for use in inhibiting FBX044 activity and/or PD-1 antibody). In particular, the compositions can exhibit inhibitory properties at concentrations that are substantially free of side effects and are therefore useful for long-term therapeutic protocols. For example, co-administration of an antibody composition with another more toxic cytotoxic drug can effectively reduce the toxic side effects of a patient while achieving beneficial inhibition of the condition or disorder being treated.

In addition, the hydrophilic and hydrophobic properties of the compositions intended for use in the present disclosure may be designed to be balanced, thereby enhancing their availability for both in vitro and especially in vivo use, but such a balance. In some embodiments, the compositions intended for use in the present disclosure can have appropriate solubility in an aqueous medium that allows absorption and bioavailability in the body, and the compound has a putative effect across the cell membrane. It also has a certain solubility in the lipids that allow it to enter the site. Therefore, the expected effectiveness of antibody compositions is maximized when they can be delivered to the target antigen active site.

### Administration and Dosing

In one aspect, the methods of the present disclosure include the step of administering a pharmaceutical composition. In certain embodiments, the pharmaceutical composition is a sterile composition. The compositions comprising the chemical entities herein can be administered by any suitable means, including parenteral, subcutaneous, intraperitoneal, intrapulmonary, and intranasal, and if topical treatment is desired, intralesional administration. Parenteral injections include intravenous, intraarterial, intraperitoneal, intramuscular, intradermal, or subcutaneous administration. In addition, the compositions described herein (e.g., FBX044 inhibitor) is preferably administered by pulse injection, especially at reduced doses (e.g., FBX044 antibody). Preferably, the dosing is given by injection, most preferably intravenously or subcutaneously, depending on whether the partial administration is short-term or long-term. Other methods of administration are also envisioned, including topical, particularly transdermal, transmucosal, rectal, oral, or topical administration, e.g., through a catheter placed near the desired site.

The methods of the present disclosure introduce therapeutic agents directly or indirectly into mammalian subjects, including but not limited to injection, ingestion, nasal, topical, transdermal, parenteral, inhalation spray, vaginal, or rectal administration. It is done using any medically acceptable means for. As used herein, the term “parenteral” includes subcutaneous, intravenous, intramuscular, and intra-incisional injections, as well as catheter or infusion methods. Intradermal, intramammary, intraperitoneal, intrathecal, post-ocular, intrapulmonary injections, or surgical transplants to specific sites are also envisioned.

In one embodiment, administration is directed to the site of the cancer or to the affected tissue in need of treatment, either by direct injection into a site where the formulation can be delivered internally, or through a sustained delivery or sustained release mechanism. For example, a biodegradable microsphere or capsule or other biodegradable polymer composition (e.g., soluble polypeptide, antibody, or small molecule) capable of sustained delivery of the composition is implanted near or to the site of the cancer can be included in the formulations of the present disclosure.

The dosing regimen is adjusted to provide the optimal desired response, e.g. a therapeutic response or minimal side effects. For example, when administering anti-FBX044 antibody or anti-PD-L1 Ab, the dosage ranges from about 0.0001 to about 100 mg/kg (body weight), typically from about 0.001 to about 20 mg/kg (body weight). In some cases, they may range more typically from about 0.01 to about 10 mg/kg (body weight). Preferably, the dosage is in the range of 0.1 to 10 mg/kg (body weight). For example, the dosage may be 0.1, 0.3, 1, 3, 5 or 10 mg/kg (body weight), more preferably 0.3, 1, 3, or 10 mg/kg (body weight). The dosing schedule is typically designed to achieve exposure resulting in sustained receptor occupancy (RO) based on the typical pharmacokinetic properties of the chemical or biological entity. In some cases, exemplary treatment regimens can be once per week, once every two weeks, once every three weeks, once every four weeks, once every month, once every three months, or once every three to six months. Entails. Dosage and scheduling can vary during the course of treatment. For example, the dosing schedule may be such that the pharmaceutical compositions (e.g., comprising FBX044 siRNA) is (i) administered every 2 weeks on a 6-week cycle; (ii) administered 6 times every 4 weeks, then every 3 months; (iii) administered every 3 weeks; (iv) 3 to 10 mg/kg (body weight) is administered once, and then 1 mg/kg (body weight) is administered every 2 to 3 weeks. In other cases, given that IgG4 Abs typically have a half-life of 2-3 weeks, the preferred dosing regimen for anti-PD-1 or anti-FBX044 Abs of the present invention may be via intravenous administration, from 0.3 to 10 mg/kg (body weight), preferably 3 to 10 mg/kg (body weight), more preferably 3 mg/kg (body weight), and such Abs are up to a cycle of 6 or 12 weeks until complete response or confirmation of progressive disease. It is administered every 14 days.

Administration of multiple agents, including, but not limited to, chemotherapeutic agents, (e.g., an FBX044 antibody compositions with a PD-1 inhibitory agent) described herein, is also envisioned herein. The amount of inhibitor or antibody composition in a given dosage may vary depending on the size of the individual being treated and the nature of the disorder being treated. In exemplary treatments, about 1 mg/day, 5 mg/day, 10 mg/day, 20 mg/day, 50 mg/day, 75 mg/day, 100 mg/day, 150 mg/day, 200 mg/day, 250 mg/day, 500 mg/day, or 1000 mg/day may need to be administered. These concentrations can be administered as a single dosage form or as multiple doses. Standard dose-response studies, first modeled on animals and then in clinical trials, reveal optimal dosages for specific disease states and patient populations.

It is also envisaged in the present disclosure that the amount of the therapeutic agents (e.g., FBX044 and PD-1 inhibitors) in a given dosage may vary depending on the size of the individual being treated and the nature of the disorder being treated. Both inhibitor compositions can be administered in doses ranging from 0.1 to 15 mg as intravenous infusions every 1 to 4 weeks for 30 to 60 minutes until disease progression or unacceptable toxicity. In various embodies, the dose can be 1, 2, 3, 4, 5, 6, 7, 8, 9, 10 mg/kg. It is also clear that dosing can be modified when conventional therapeutic agents are administered in combination with the therapeutic agents of the present disclosure.

In some methods, two or more mAbs (for example, FBX044 antibody and PD-1 antibody) with different binding specificities may be administered simultaneously, in which case the dosage of each Ab administered falls within the indicated range. Antibodies are usually administered in multiple times. The interval between single administrations can be, for example, weekly, every two weeks, every three weeks, every month, every three months or every year. The interval can also be irregular as indicated by measuring the blood level of the pharmaceutical entity (for example, FBX044 antibody or FBX044 siRNA) in the patient. In some methods, the dosage is adjusted to achieve a plasma Ab concentration of about 1 to 1,000 μg/mL, and in some methods a plasma Ab concentration of about 25 to 300 μg/mL. In some methods, the dosage is adjusted to achieve a plasma siRNA concentration of about 1 to 1,000 μg/mL, and in some methods a plasma siRNA concentration of about 25 to 300 μg/mL.

On the other hand, the composition comprising the therapeutic agent (e.g., the FBX044 Antibody) can be administered as a sustained release formulation, in which case less frequent administration is required. Dosage and frequency vary depending on the half-life of Ab in the patient. In general, human Abs exhibit the longest half-life, followed by humanized Abs, chimeric Abs and non-human Abs. The dosage and frequency of administration can vary depending on whether the treatment is prophylactic or therapeutic. In prophylactic applications, relatively low dosages are typically administered at relatively sparse intervals over an extended period of time. Some patients continue to receive treatment for the rest of his life. In therapeutic applications, it is often required to administer relatively high doses at relatively short intervals until the progression of the disease is reduced or terminated, preferably until the patient shows partial or complete recovery of disease symptoms. Thereafter, the patient can be administered a prophylactic regimen.

The therapeutic composition can also be delivered to the patient at multiple sites. Multiple doses may be provided simultaneously or may be administered over a period of time. In certain cases, it is beneficial to provide a continuous stream of therapeutic composition. Periodically, for example, once an hour, once a day, once every two days, twice a week, three times a week, once a week, every other week, once every three weeks, once a month, or Additional therapy can be given at longer intervals.

The actual dosage level of the active ingredient in the pharmaceutical composition of the present invention can be varied to obtain an amount of active ingredient effective to achieve the desired therapeutic response to the particular patient, composition and mode of administration, while not being excessively toxic to the patient. The selected dosage level depends on the activity of the particular composition of the invention employed, the route of administration, the time of administration, the rate of excretion of the particular compound employed, the duration of treatment, and other drugs, compounds and/or substances used in combination with the particular composition employed. The age, sex, weight, condition, general health and previous medical history of the patient being treated, and other factors well known in the medical field. The composition of the present invention can be administered through one or more routes of administration using one or more of various methods well known in the art. As will be appreciated by those skilled in the art, the route and/or mode of administration will vary depending on the desired outcome.

Measurement of Effects of Treatment with Pharmaceutical Compositions

The methods herein are expected to reduce tumor size or tumor loading in a subject and/or reduce metastasis in a subject. In various embodiments, the method reduces tumor size by 10%, 20%, 30% or more. In various embodiments, the methods herein reduce tumor size by 10%, 15%, 20%, 25%, 30%, 35%, 40%, 45%, 50%, 55%, 60%, 65%, 70%, 75%, 80%, 85%, 90%, 95%, or 100% reduction.

The methods herein are also expected to reduce tumor burden and reduce or prevent tumor recurrence once the cancer is in remission.

In various embodiments, the FBX044 and/or PD-1 inhibitors described herein, as well as combinations or compositions thereof, regulate immune cells in tumors. In some embodiments, the FBX044 and/or PD-1 inhibitors herein, as well as combinations or compositions thereof, increase the number of natural killer (NK) cells in tumors and/or NK cells. In some embodiments, the FBX044 and/or PD-1 inhibitors herein, as well as combinations or compositions thereof, increase the cytolytic activity of natural killer (NK) cells in tumors. In various embodiments, the FBX044 inhibitors or compositions described herein reduce the number of regulatory T cells in a tumor and/or inhibit regulatory T cell function. For example, in various embodiments, the FBX044 or compositions described herein inhibit the Treg's ability to down-regulate the immune response or migrate to the site of the immune response.

In various embodiments, the FBX044 and/or PD-1 FBX044 described herein, and combinations or compositions thereof, increase the number of cytotoxic T cells in the tumor and/or enhances activity, e.g., boosts, increases, or promotes CTL activity. For example, in various embodiments, the FBX044 inhibitors or compositions described herein increase perforin and granzyme production by CTL and increase the cytolytic activity of CTL.

In various embodiments, the FBX044 and/or PD-1 inhibitors described herein, and combinations or compositions thereof, increase the number of dendritic cells (DCs) in the tumor and/or inhibits the immunotolerant function of dendritic cells (e.g., immunotolerant action). For example, in various embodiments, the inhibitors or compositions described herein reduce the immunotolerant effect of CD8+dendritic cells.

### Kits

As an additional aspect, the disclosure includes a kit comprising one or more compounds or compositions packaged in a manner that facilitates the use of the compounds or compositions to practice the methods of the present disclosure. In one embodiment, such kit is packaged in a container, such as a sealed bottle or tube, as described herein in a compound or composition (e.g., alone or in combination with another antibody or third agent). A label describing the use of the compound or composition in the practice of the method is affixed to the container or included in the package. Preferably, the compound or composition is packaged in unit dosage form. The kit may further include a device suitable for administration of the composition by a specific route of administration or for practicing a screening assay. Preferably, the kit comprises a label describing the use of the inhibitor composition.

Additional aspects and details of the present disclosure will be apparent from the following implementations, which are not intended to be limiting and are intended to be illustrated.

### EXAMPLES

The following examples are included for illustrative purposes only and are not intended to limit the scope of the invention.

### Example 1: Identification of Regulators of H3K9me3 in Cancer Cells

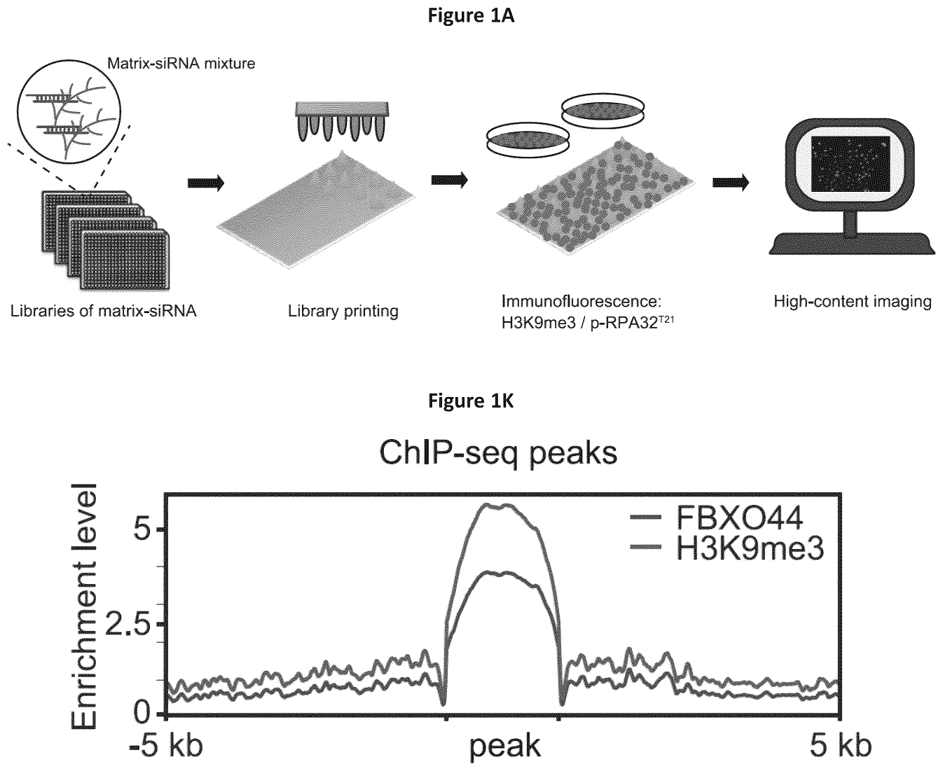

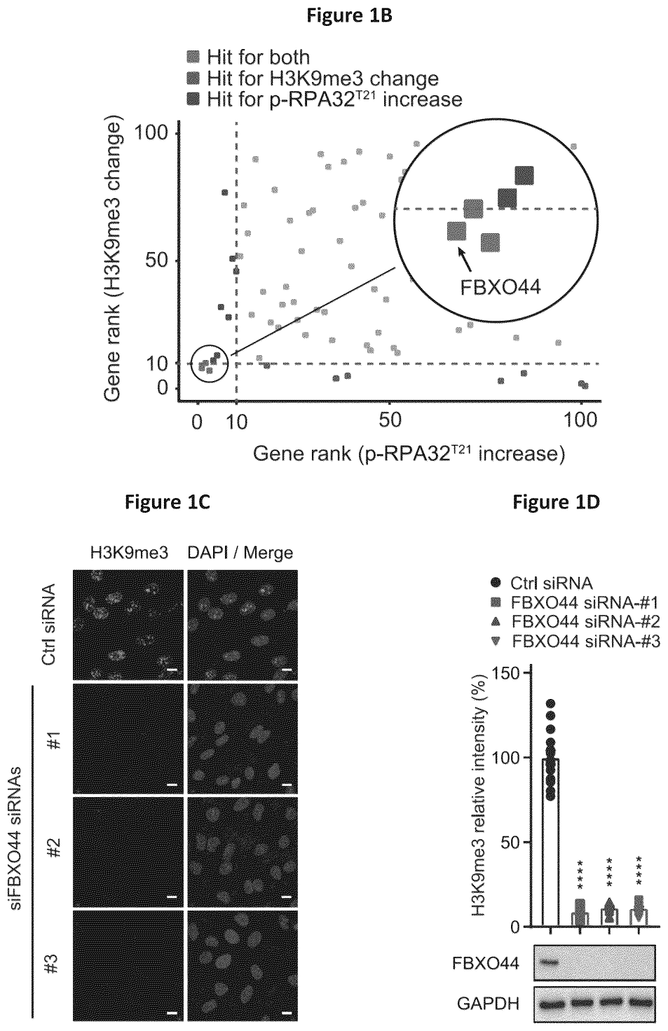

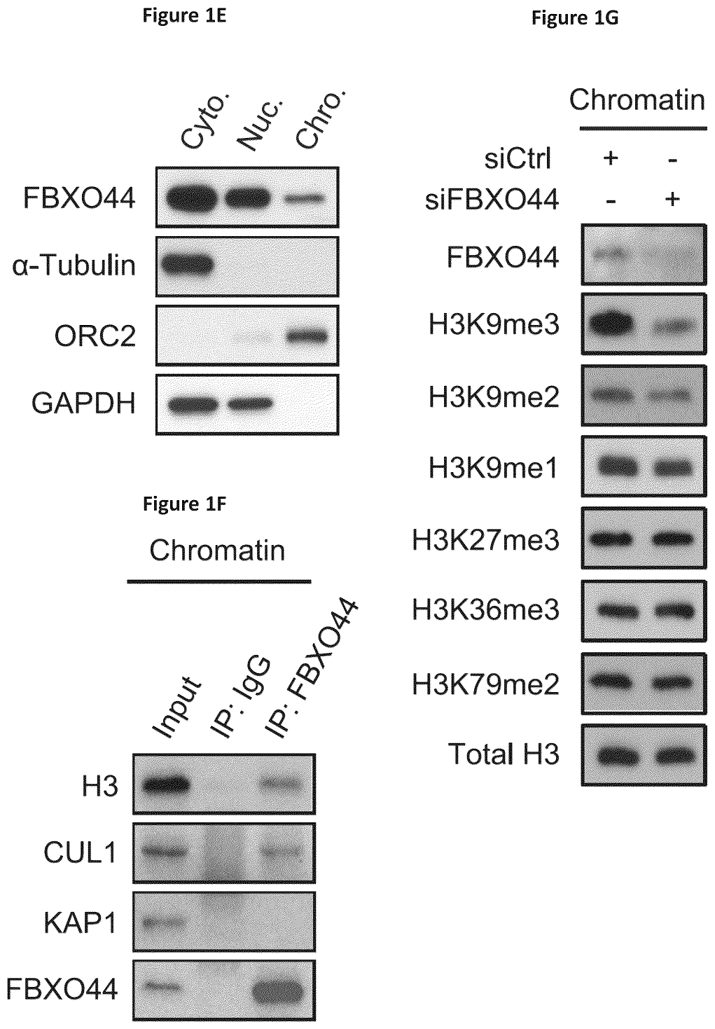

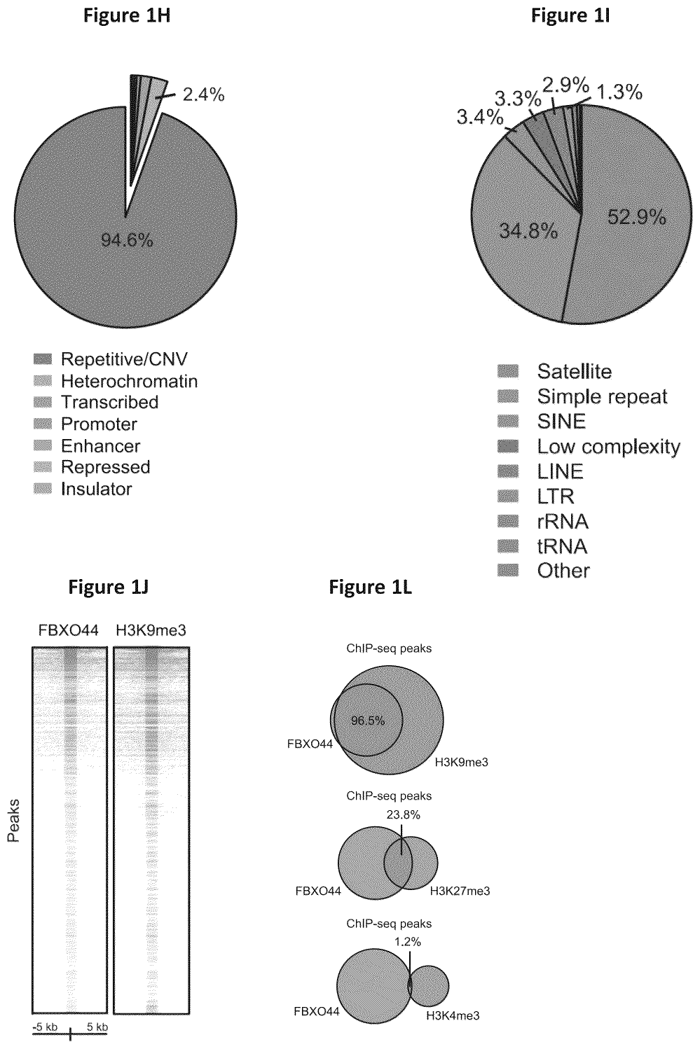

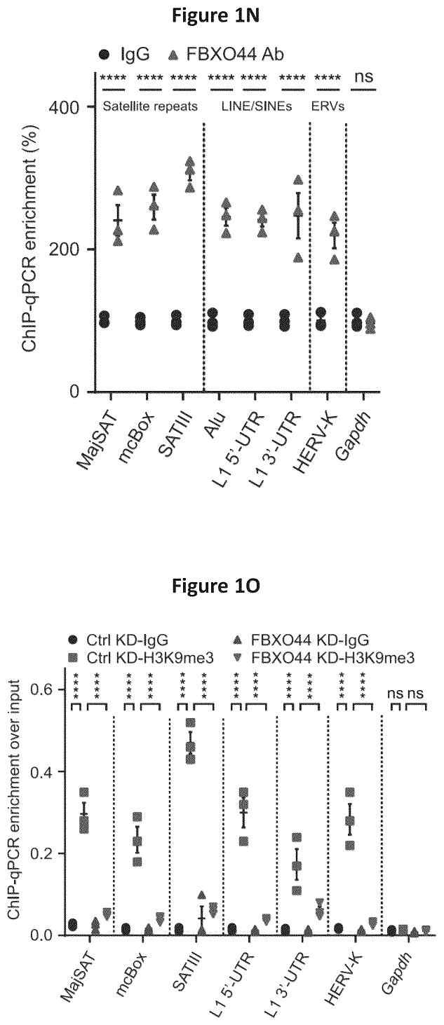

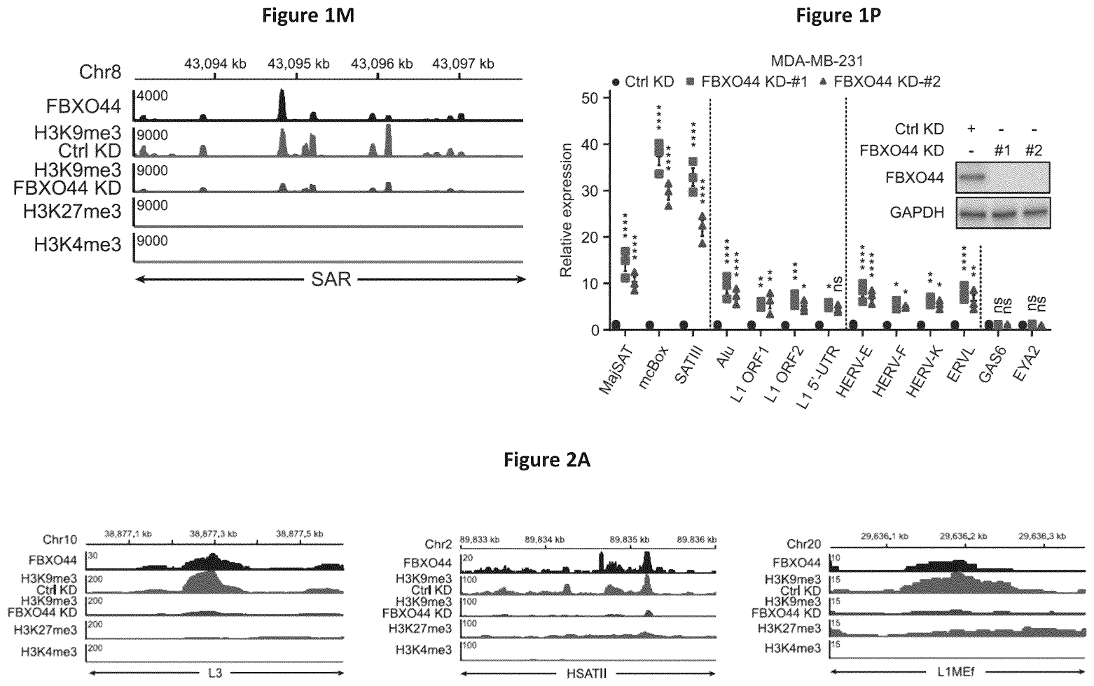

To identify H3K9me3 regulators, a quantitative imaged-based RNA interference (RNAi) screen was performed in a panel of cancer cell lines, using a cell-spot microarray approach, modified for detection of DAPI, p-RPA32^{T21}, and H3K9me3 (FIG. 1A).

Cell lines and culture conditions: all cancer cell lines were purchased from the American Tissue Culture Collection (ATCC) and grown in recommended culture media. Specifically, MDA-MB-231, BT-549, MCF7, A549, and H446 cells were cultured in RPMI 1640 (Corning), HEK293T, HEK293FT, and HeLa cells were cultured in DMEM (Corning), MDA-MB-231-luc cells were cultured in MEM (Corning), U2OS and HCT116 cells were cultured in McCoy's 5A (Corning). The above media were supplemented with 10% fetal bovine serum (FBS), 2 mM glutamine, and 100 U/mL penicillin and streptomycin. Patient-derived glioma stem cells (GSCs) were obtained (Xie et al., 2018) and cultured in Neurobasal medium (Thermo Fisher Scientific, #11360070) supplemented with B27 without vitamin A (Thermo Fisher Scientific, #A3353501), EGF, and bFGF (20 ng/mL each; R&D Systems), sodium pyruvate (Thermo Fisher Scientific, #11360070), and GlutaMAX (Thermo Fisher Scientific, #35050061). All cells were incubated at 37° C. in 5% CO₂. The cell cultures were authenticated by short tandem repeat (STR) analysis.

Gene silencing: FBXO44 short interfering RNA (siRNAs) (no. SI00145663, SI00145670, SI03078551) were purchased from Qiagen, with SI00145663 and SI00145670 used in most experiments. Other siRNAs used included SUV39H1 (Qiagen no. SI00048685 and SI02665019), GATAD2A (Qiagen no. SI04318636), GATAD2B (Dharmacon no. J-013892-06-0005), CHD4 (Qiagen no. SI00024563), CUL4B (Qiagen no. SI04215015 and SI04292540), CUL1 (Qiagen no. SI02225657 and SI02225664), RBBP4 (Dharmacon no. L-012137-00-0005), RBBP7 (Dharmacon no. L-011375-00-0005), IRF3 (Qiagen no. SI02626526), and IRF7 (Qiagen no. SI00448672). siRNA transfections were performed using Lipofectamine RNAiMAX Reagent (Thermo Fisher Scientific). Unless otherwise noted, cells were harvested 72 hrs post-transfection. A cell-spot microarray approach (Rantala et al., 2011) was modified for detection of DAPI, p-RPA32T21, and H3K9me3.

Results from siRNA-mediated knockdown (KD) showed that among the top hits, KD of FBXO44 decreased chromatin-associated H3K9me3 modifications and increased p-RPA32T21 levels (FIG. 1B).

Immunofluorescence (IF): IF was performed to detect chromatin associated H3K9me3 in siRNA of FBXO44 or control. Briefly, cells were grown on Laminin-coated coverslips (neuVitro no. GG-12-laminin), washed twice with cold PBS, and then fixed with 4% paraformaldehyde for 20 min at 25° C. followed by permeabilization with 0.5% Triton X-100 in PBS for 5 min. Cells were fixed with chilled methanol for 5 min at −20° C. After 3×10 min washes in PBS, cells were incubated in blocking buffer (10 mM Tris-Cl [pH 7.5], 120 mM KCl, 20 mM NaCl, 0.5 mM EDTA, 0.1% Triton X-100, 10% milk, 2% BSA) overnight at 4° C. The relevant primary antibodies diluted in blocking buffer were then added. Specifically, 1:500 dilution for anti-H3K9me3 (Abcam no. ab8898). Cells were washed 3×10 min with KCMT buffer (10 mM Tris-Cl [pH 7.5], 120 mM KCl, 20 mM NaCl, 0.5 mM EDTA, 0.1% Triton X-100) and incubated with Alexa Fluor 488-labeled anti-rabbit (Thermo Fisher Scientific no. A-21441) or Alexa Fluor 594-labeled anti-mouse (Cell Signaling Technologies no. 8890) antibodies at 1:500 dilution for 1 hr at 25° C. After 3×10 min washes with KCMT buffer, cells were stained with DAPI (Thermo Fisher Scientific no. D1306), coverslips mounted with anti-fade mounting medium (Vector Laboratories no. H-1000), and slides examined by fluorescence microscopy (Nikon Inverted TE300) or confocal microscopy (Zeiss LSM 710 NLO).

IF results of siRNA-mediated KD of FBXO44 in MDA-MB-231 cells showed a decrease of chromatin-associated H3K9me3 (FIG. 1C).

Immuneblotting: immunoblotting was performed to confirm that the siRNA-mediated knockdown (KD) of FBXO44 decreased chromatin-associated H3K9me3 modifications levels. Briefly, cells were lysed in cold RIPA buffer (50 mM Tris-HCl [pH 7.4], 150 mM NaCl, 1 mM EDTA, 1% NP-40, 0.01% SDS) supplemented with protease and phosphatase inhibitors (50 mM NaF, 1 mM PMSF, 1 mM Na₃VO₄, 1 μg/mL Aprotinin, 1 μg/mL Leupeptin, 1 μg/mL Pepstatin). Lysates were briefly sonicated, clarified, then subjected to SDS-PAGE and transferred to PVDF membranes using a Bio-Rad transfer apparatus. Membranes were blocked with 5% non-fat milk or BSA in TBS containing 0.1% Tween-20 (TBST) at 25° C. for 1 hr, followed by incubation with primary antibody overnight at 4° C. Membranes were washed 3×10 min in TBST and incubated with species-specific HRP-conjugated secondary antibodies (Pierce) for 1 hr at 25° C. After 3×10 min washes in TBST, membranes were developed using an enhanced chemiluminescence (ECL) reagent before being exposed to film or ChemiDoc Imaging System (Bio-Rad).

IF results showed that siRNA-mediated KD of FBXO44 decreased chromatin-associated H3K9me3 modifications (FIG. 1D).

### Example 2: Identification of FBXO44 in Chromatin-Bound Fractions of DNA

In order to detect FBXO44 in cytoplasmic, nuclear, and chromatin-bound fractions, fractionation followed by immunoblotting for FBXO44 was performed.

Sample preparation: for direct western blotting, chromatin was extracted using a commercial kit (Abcam no. ab117152), sonicated in RIPA buffer, and dissolved in 4×SDS loading buffer. For chromatin fractionation experiments, cells were resuspended in buffer A (10 mM HEPES [pH 7.9], 10 mM KCl, 1.5 mM MgCl2, 0.1% Triton X-100, 0.34 M sucrose, 10% glycerol, 1 mM DTT, 50 mM NaF, 1 mM PMSF, 1 mM Na₃VO₄, 1 μg/mL Aprotinin, 1 μg/mL Leupeptin, 1 μg/mL Pepstatin) and incubated on ice for 5 min, followed by centrifugation at 1,300×g for 5 min. The cytoplasmic supernatant was then collected. The nuclei pellet was washed once in buffer A and lysed in buffer B (3 mM EDTA, 0.2 mM EGTA, 1 mM DTT, with protease/phosphatase inhibitors described above) on ice for 30 min. After centrifugation at 12,000×g for 20 min, the nucleoplasmic supernatant was collected. The insoluble chromatin pellet was then washed once with buffer B and centrifuged as described above. The chromatin pellet was then resuspended in RIPA buffer for 20 min at 4° C. and sonicated extensively to release chromatin-bound proteins. After centrifugation at 12,000×g for 20 min, chromatin-associated proteins were collected for electrophoresis. Immunoblot was performed as described in example 1.

Results from fractionation experiments showed that FBXO44 was detected in the cytoplasm, nucleus, and bound to chromatin (FIG. 1E).

### Example 3: Identification of Histone H3 Interaction with FBXO44

To investigate protein interaction with FBXO44 on chromatin, immunoprecipitation (IP) and immunoblotting of chromatin fraction were performed.

Immunoprecipitation or co-immunoprecipitation (IP or co-IP): for IP, cells were lysed in IP buffer (50 mM Tris-HCl [pH 7.4], 125 mM NaCl, 1 mM EDTA, 0.1% Triton X-100) with inhibitors (50 mM NaF, 1 mM PMSF, 1 mM Na₃VO₄, 1 μg/mL Aprotinin, 1 μg/mL Leupeptin, 1 μg/mL Pepstatin) followed by sonication and centrifugation to clear insoluble debris. Lysates were then incubated with protein G agarose beads and IgG antibody of the same species as the IP antibody for 2 hr at 4° C. to reduce non-specific binding. The cleared lysates were then incubated with IP antibody overnight at 4° C. Specifically, for Flag IP, 5 μg of anti-Flag antibody (Sigma-Aldrich no. F7425) was used for 2500 μg of protein lysate. For Myc IP, anti-Myc antibody (Cell Signaling Technology no. 2276) was used at 1:1000 dilution. For FBXO44 IP, 5 μL of anti-FBXO44 antibody (Sigma-Aldrich no. HPA003363) was used for 10⁶ cells. Beads were washed with IP buffer, boiled in 1×SDS gel loading buffer, and subjected to electrophoresis.

Results from immunoprecipitation (IP) verified FBXO44 interacted with Histone H3 in chromatin fractions (FIG. 1F). Immunoblotting of chromatin fraction was performed as described in example 2. Immunoblotting results confirmed that FBXO44 KD reduced chromatin-associated H3K9me3, and to a lesser extent H3K9me1 and H3K9me2 modifications (FIG. 1G). H3K27me3, H3K36me3, and H3K79me2 modifications were unaffected.

### Example 4: Identification of Repetitive Elements (RE) Associated with FBXO44

To investigate genomic sequence associated with FBXO44, chromatin IP sequencing (ChIP-seq), ChIP-qPCR, and quantitative (q)RT-PCR were performed.

ChIP-seq: libraries were made using the NEBNext Ultra II DNA Library Prep Kit for Illumina (NEB). Libraries were pooled and single end sequenced (1×75) on an Illumina NextSeq 500 using the High Output V2 kit (Illumina). Read data was processed in BaseSpace (basespace.illumina.com). ChIP-seq reads were then aligned to the Homo sapiens genome (hg19) using Bowtie 2 with default parameters, which allows multiple alignment. Peaks were called using Partek software with default parameters. The midpoint of each estimated fragment by Partek and its location on the genome was calculated. The genome was divided into non-overlapping windows of the default 100 bp. An aligned read was considered to be located in a window if the midpoint of its estimated fragment was within the window. The number of midpoints in each window was counted and an empirical distribution of window counts was created. A zero-truncated negative binomial model was fit to the distribution, and a peak was determined based on the FDR (0.001, default) calculated from the model. Overlapping enriched windows were merged into regions and reported. For analysis of REs from ChIP-seq data, a list of FBXO44 peaks associated with REs was created by intersecting FBXO44 binding peaks with RE loci obtained from RepeatMasker, which was used in the downstream analysis. Reads were mapped and assigned to the FBXO44-associated REs using RepEnrich2 with the recommended parameters (https://github.com/nerettilab/RepEnrich2). The resulting counts for REs were analyzed by the edgeR package to obtain CPM values. The H3K27me3 and H3K4me3 ChIP-seq data in MDA-MB-231 cells were obtained from GSE87169 (Lee et al., 2016). The BigWig files were obtained using deeptools bamCoverage command with—normalizeUsing CPM. The heatmap and profile plots for ChIP-seq data were performed by deeptools plotHeatmap and plotProfile in the scale-regions mode. Venn diagrams were obtained using VennDiagram package. The ChIP-seq binding signal from BAM files was visualized in The Integrative Genomics Viewer (IGV).

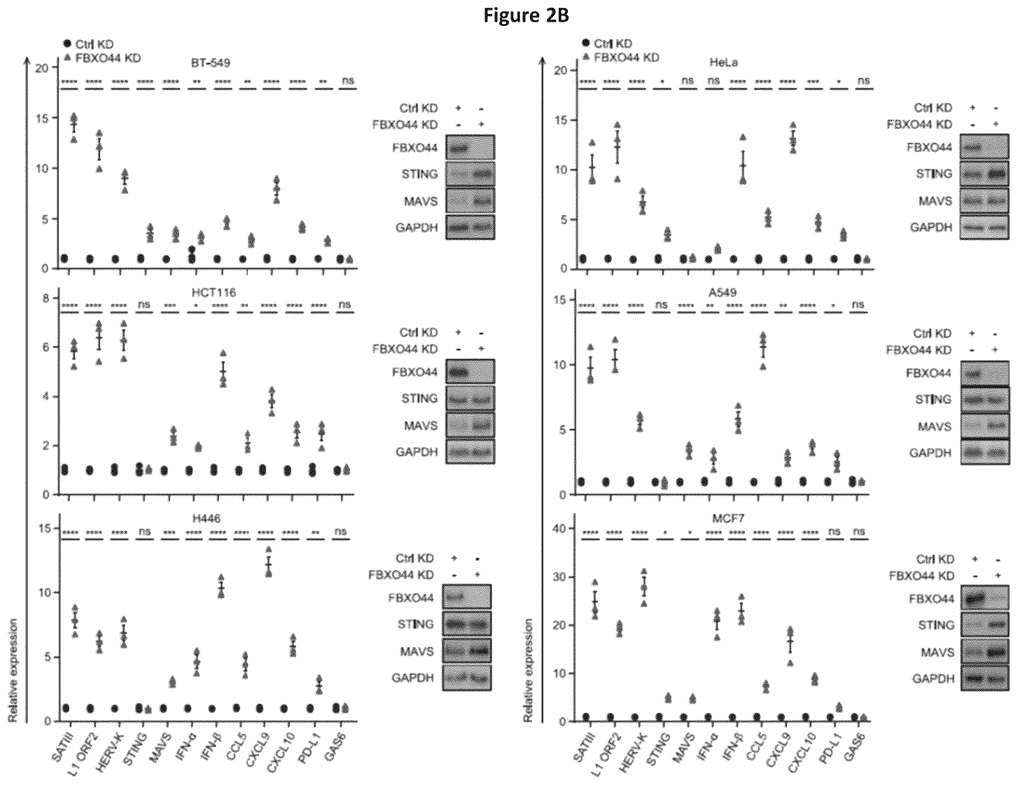

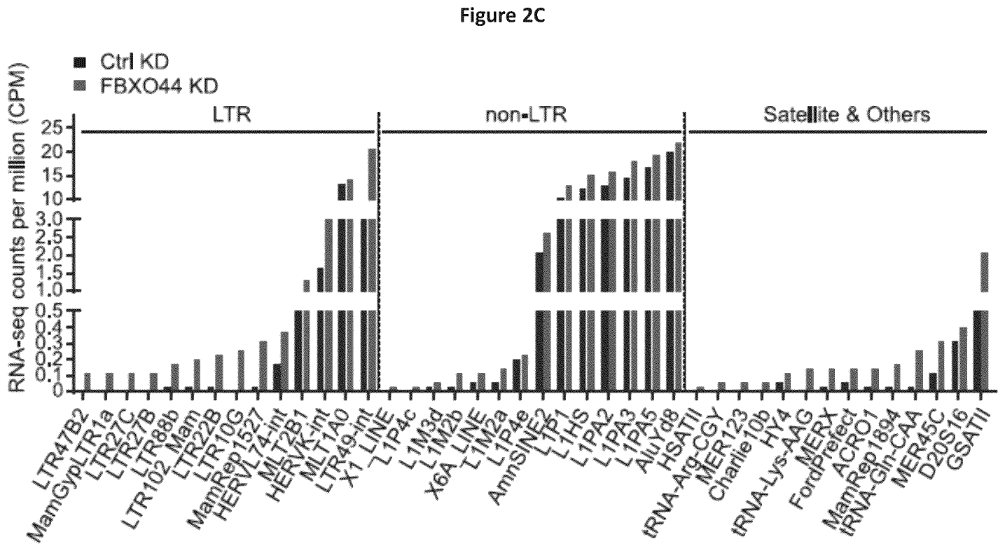

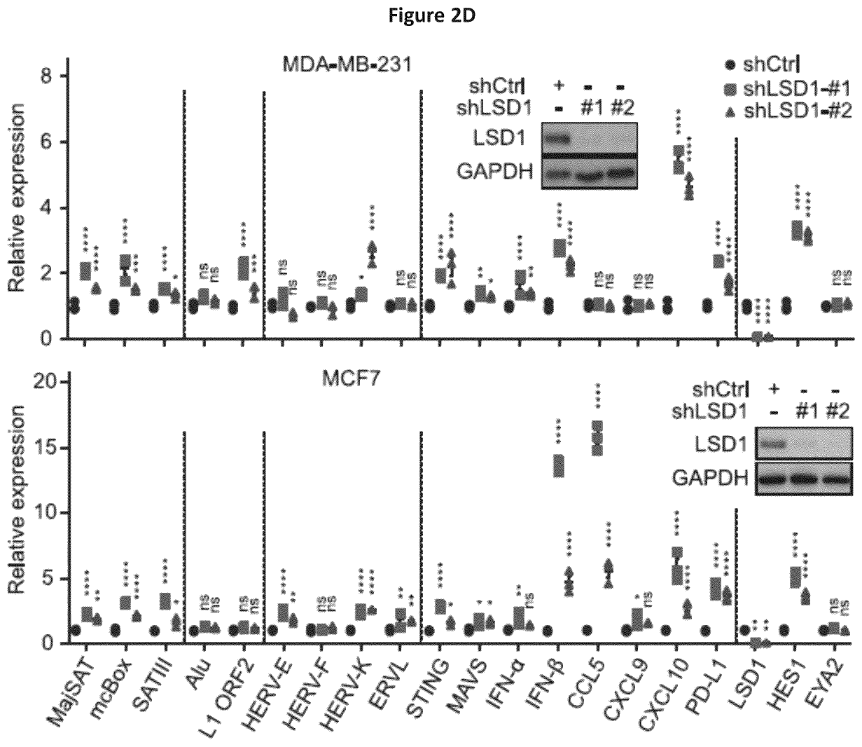

Results from ChIP-seq revealed that FBXO44 was highly enriched at repetitive DNA (94.6%) and heterochromatin (2.4%), with little binding at promoters, enhancers, or transcribed regions (<3% total; FIG. 1H). Specifically, FBXO44 binding localized to various RE subtypes (FIG. 1I). FBXO44 chromatin binding sites strongly co-localized with H3K9me3, and much less with H3K27me3 or H3K4me3 modifications, across the genome and specifically at REs (FIGS. 1J-1M; FIG. 2A).

ChIP-qPCR: for Flag and FBXO44 ChIP experiments, cells were cross-linked with 1% formaldehyde for 10 min at 25° C. and reactions stopped by adding glycine to a final concentration of 0.125 M for 5 min at 25° C. Cells were rinsed 3× with PBS and re-suspended in ChIP lysis buffer (50 mM Tris-HCl [pH 8.1], 1% SDS, 10 mM EDTA, 0.2 mM PMSF, 1 μg/mL Aprotinin, 1 μg/mL Leupeptin). Specifically, lysates prepared from 106 cells in 130 μL lysis buffer were introduced into micro sonication tubes (Covaris no. 520045). For H3K9me3 ChIP experiments, native ChIP was performed. Cells were harvested and washed with cold PBS and native chromatin extracted using a commercial chromatin extraction kit (Abcam no. ab117152). For H2AK119ub native ChIP, 1 μL of anti-H2AK119ub antibody (Cell Signaling Technology no. 8240) was used for 106 cells. For all ChIP experiments, DNA was fragmented by sonication in a S220 Focused-Ultrasonicator (Covaris) for 7 min (Duty cycle—5%, Intensity—4, Cycles/Burst—200). After centrifugation, the supernatant was diluted 1:10 by adding ChIP dilution buffer (Tris-HCl [pH 8.1], 0.01% SDS, 1.1% Triton X-100, 1.2 mM EDTA, 16.7 mM NaCl, 0.2 mM PMSF, 1 μg/mL Aprotinin, 1 μg/mL Leupeptin), 5% saved for input, and the remaining lysate pre-cleared for 2 hrs with 15 μl protein G agarose before overnight incubation with the indicated antibody. Specifically, for Flag ChIP, 2.5 μL of anti-Flag antibody (Cell Signaling Technology no. 14793) was used for 106 cells. For FBXO44 ChIP, 5 μL of anti-FBXO44 antibody (Sigma-Aldrich no. HPA003363) was used for 106 cells. For SUV39H1 ChIP, 6 μL of anti-SUV39H1 antibody (Millipore no. 05-615) was used for 106 cells. For γH2AX ChIP, 4 μL of anti-γH2AX antibody (Abeam no. 2893) was used for 106 cells. For H3K9me3 ChIP, the ChIPAb+Trimethyl-Histone H3 (Lys9) kit (Millipore no. 17-10242) was used, typically with 1 μL of anti-H3K9me3 antibody for 106 cells. Bound material was recovered after incubation with 15 μL of protein G beads at 4° C. Beads were then washed sequentially with low salt buffer (20 mM Tris-HCl [pH 8.1], 0.1% SDS, 1% Triton X-100, 2 mM EDTA, 150 mM NaCl), high salt buffer (20 mM Tris-HCl [pH 8.1], 0.1% SDS, 1% Triton X-100, 2 mM EDTA, 500 mM NaCl), LiCl buffer (10 mM Tris-HCl [pH 8.1], 0.25 M LiCl, 1% IGEPAL-CH630, 1% Na deoxycholate, 1 mM EDTA) and TE (10 mM Tris-HCl [pH 8.1], 1 mM EDTA). Elution was performed by adding 200 μL of freshly prepared elution buffer (1% SDS, 0.1 M NaHCO₃) to 15 μL protein G beads and rotating the sample for 15 min at 25° C. After centrifugation, the chromatin IP and input were reverse-cross-linked by adding 5M NaCl (4 for input and 8 μL for IP sample) and incubated overnight at 65° C., and then DNA extracted using the Gel Extraction Kit (Qiagen no. 28704). Primers used included;

Maj SAT-
(SEQ ID NO: 1)
GGCGAGAAAACTGAAAATCACG
and
(SEQ ID NO: 2)
CTTGCCATATTCCACGTCCT,
mcBox-
(SEQ ID NO: 3)
AGGGAATGTCTTCCCATAAAAACT
and
(SEQ ID NO: 4)
GTCTACCTTTTATTTGAATTCCCG,
SATIII-
(SEQ ID NO: 5)
AATCAACCCGAGTGCAATC[[N]]GAATGGAATCG
and
(SEQ ID NO: 6)
TCCATTCCATTCCTGTACTCGG,
ALU-
(SEQ ID NO: 7)
AATGGTACGATCTCGGCTCA
and
(SEQ ID NO: 8)
TAGCCAGGTGTGGTGACTTG,
L1 5′UTR-
(SEQ ID NO: 9)
GATGATGGTGATGTACAGATGGG
and
(SEQ ID NO: 10)
AGCCTAACTGGGAGGCACCC,
L1 3′UTR-
(SEQ ID NO: 11)
TGTATACATGTGCCATGCTGGTGC
and
(SEQ ID NO: 12)
AATGAGATCACATGGACACAGGAAG,
HERV-K-
(SEQ ID NO: 13)
AGAGGAAGGAATGCCTCTTGCAGT
and
(SEQ ID NO: 14)
TTACAAAGCAGTATTGCTGCCCGC,
and
Gapdh-
(SEQ ID NO: 15)
TCGAACAGGAGGAGCAGAGAG
and
(SEQ ID NO: 16)
TACTAGCGGTTTTACGGGCG.

Results from ChIP-qPCR experiments confirmed enrichment of FBXO44 bound at various subfamilies of satellite repeats (Maj SAT, mcBox, SATIII), SINE/LINEs (Alu, L1), and ERV (HERV-K) (FIG. 1N). FBXO44 targeting diminished H3K9me3 modifications at these REs (FIGS. 1M-10, and FIG. 2A).

Quantitative (q)RT-PCR: total RNA was isolated using the RNeasy Plus Mini Kit (Qiagen no. 74134). cDNA was synthesized using qScript cDNA SuperMix (Quantabio no. 95048) following the manufacturer's instructions. qPCR reactions were performed using SYBR Select Master Mix (Thermo Fisher Scientific no. 4472908) and a Stratagene Mx3000P instrument (Agilent Technologies). Thermal cycling conditions included an initial denaturation step of 95° C. for 10 min and 40 cycles at 95° C. for 15 sec and 60° C. for 60 sec. Analyses were carried out in triplicate for each data point. The qPCR primers used for gene expression analysis of human REs and genes included MajSAT-GGCGAGAAAACTGAAAATCACG (SEQ ID NO: 1) and CTTGCCATATTCCACGTCCT (SEQ ID NO: 2), mcBox-AGGGAATGTCTTCCCATAAAAACT (SEQ ID NO: 3) and GTCTACCTTTTATTTGAATTCCCG (SEQ ID NO: 4), SATIII-AATCAACCCGAGTGCAATCGAATGGAATCG (SEQ ID NO: 5) and TCCATTCCATTCCTGTACTCGG (SEQ ID NO: 6), Alu-AATGGTACGATCTCGGCTCA (SEQ ID NO: 7) and TAGCCAGGTGTGGTGACTTG (SEQ ID NO: 8), L1 ORF1-TGGCCCCCACTCTCTTCT (SEQ ID NO: 18) and TCAAAGGAAAGCCCATCAGACTA (SEQ ID NO: 19), L1 ORF2-GCCATTGCTTTTGGTGTTTT (SEQ ID NO: 20) and AAATGGTGCTGGGAAAACTG (SEQ ID NO: 21), L1 5′-UTR-AAGCAAGCCTGGGCAATG (SEQ ID NO: 22) and ACGGAATCTCGCTGATTGCTA (SEQ ID NO: 23), HERV-E-GGTGTCACTACTCAATACAC (SEQ ID NO: 24) and GCAGCCTAGGTCTCTGG (SEQ ID NO: 25), HERV-F-CCTCCAGTCACAACAACTC (SEQ ID NO: 26) and TATTGAAGAAGGCGGCTGG (SEQ ID NO: 27), HERV-K-CAGTCAAAATATGGACGGATGGT (SEQ ID NO: 28) and ATTGGCAACACCGTATTCTGCT (SEQ ID NO: 29), ERVL-ATATCCTGCCTGGATGGGGT (SEQ ID NO: 30) and GAGCTTCTTAGTCCTCCTGTGT (SEQ ID NO: 31), GAPDH-CGACCACTTTGTCAAGCTCA (SEQ ID NO: 32) and AGGGGTCTACATGGCAACTG (SEQ ID NO: 33), FBXO44-GCCCAGTAATGAGTGTCCACG (SEQ ID NO: 34) and AGATTGCGGGTCCAAGTACC (SEQ ID NO: 35), SUV39H1-CCTGCCCTCGGTATCTCTAAG (SEQ ID NO: 36) and ATATCCACGCCATTTCACCAG (SEQ ID NO: 37), IFN-α-AATGACAGAATTCATGAAAGCGT (SEQ ID NO: 38) and GGAGGTTGTCAGAGCAGA (SEQ ID NO: 39), IFN-β-GCCATCAGTCACTTAAACAGC (SEQ ID NO: 40) and GAAACTGAAGATCTCCTAGCCT (SEQ ID NO: 41), MX1-CTGCACAGGTTGTTCTCAGC (SEQ ID NO: 42) and GTTTCCGAAGTGGACATCGCA (SEQ ID NO: 43), IRF3-CATGTCCTCCACCAAGTCCT (SEQ ID NO: 44) and GGCTTGTGATGGTCAAGGTT (SEQ ID NO: 45), IRF7-TCAACACCTGTGACTTCATGT (SEQ ID NO: 46) and GTGGACTGAGGGCTTGTA (SEQ ID NO: 47), CCL5-CCAGCAGTCGTCTTTGTCAC (SEQ ID NO: 48) and CTCTGGGTTGGCACACACTT (SEQ ID NO: 49), CXCL9-CCAGTAGTGAGAAAGGGTCGC (SEQ ID NO: 50) and AGGGCTTGGGGCAAATTGTT (SEQ ID NO: 51), CXCL10-GCCTTCGATTCTGGATTCAG (SEQ ID NO: 52) and GTGGCATTCAAGGAGTACCTC (SEQ ID NO: 53), cGAS-GCCGCCGTGGAGATATCAT (SEQ ID NO: 54) and GGCGGTTTTGGAGAAGTTGA (SEQ ID NO: 55), STING-ATATCTGCGGCTGATCCTGC (SEQ ID NO: 56) and TTGTAAGTTCGAATCCGGGC (SEQ ID NO: 57), RIG-I, CCAGCATTACTAGTCAGAAGGAA (SEQ ID NO: 58) and CACAGTGCAATCTTGTCATCC (SEQ ID NO: 59), MAVS-CAGAACTGGGCAGTACCC (SEQ ID NO: 60) and AGGAGACAGATGGAGACACA (SEQ ID NO: 61), IFNGR1-TTCCATCTCGGCATACAGCAA (SEQ ID NO: 62) and TCTTTGGGTCAGAGTTAAAGCCA (SEQ ID NO: 63), IFNGR2-CTCCTCAGCACCCGAAGATTC (SEQ ID NO: 64) and GCCGTGAACCATTTACTGTCG (SEQ ID NO: 65), PTPN2-TGCAGTTTAACACGACTGTGA (SEQ ID NO: 66) and GAAGAGTTGGATACTCAGCGTC (SEQ ID NO: 67), PD-L1-GTGGCATCCAAGATACAAACTCAA (SEQ ID NO: 68) and TCCTTCCTCTTGTCACGCTCA (SEQ ID NO: 69), GAS6-GGTAGCTGAGTTTGACTTCCG (SEQ ID NO: 70) and GACAGCATCCCTGTTGACCTT (SEQ ID NO: 71), EYA2-CAGCGATTGTCTGGATAAACTGA (SEQ ID NO: 72) and GGAGGTGGGTAAGCTGTATAGG (SEQ ID NO: 73), HES1-CGTGCGAGGGCGTTAATA (SEQ ID NO: 74) and GGGTAGGTCATGGCATTGAT (SEQ ID NO: 75), and LSD1-GTGGACGAGTTGCCACATTTC (SEQ ID NO: 76) and TGACCACAGCCATAGGATTCC (SEQ ID NO: 77). qPCR primers used for analysis of mouse REs and genes included FBXO44-TACCTTCCATTCATCGCCTCC (SEQ ID NO: 78) and CATTGACCTGGTTACACTCTGG (SEQ ID NO: 79), SUV39H1-TGTCAACCATAGTTGTGATCC (SEQ ID NO: 80) and GCATGTTGTAATCAAAGGTGAG (SEQ ID NO: 81), Maj SAT II-CTTGCCATATTCCACGTCCT (SEQ ID NO: 2) and GGCGAGAAAACTGAAAATCACG (SEQ ID NO: 1), MinSAT II-TTGGAAACGGGATTTGTAGA (SEQ ID NO: 82) and CGGTTTCCAACATATGTGTTTT (SEQ ID NO: 83), LINE1-CTGCCGTCTACTCCTCTTGG (SEQ ID NO: 84) and TTTGGGACACAATGAAAGCA (SEQ ID NO: 85), MERV-L-GACACCTTTTTTAACTATGCGAGCT (SEQ ID NO: 86) and TTTCTCAAGGCCCACCAATAGT (SEQ ID NO: 87), STING-GGTCACCGCTCCAAATATGTAG (SEQ ID NO: 88) and CAGTAGTCCAAGTTCGTGCGA (SEQ ID NO: 89), MAVS-CTGCCTCACAGCTAGTGACC (SEQ ID NO: 90) and CCGGCGCTGGAGATTATTG (SEQ ID NO: 91), IFN-α-CGGTGCTGAGCTACTGGC (SEQ ID NO: 92) and TTTGTACCAGGAGTGTCAAGG (SEQ ID NO: 93), IFN-β-GGTGGAATGAGACTATTGTTG (SEQ ID NO: 94) and AGGACATCTCCCACGTC (SEQ ID NO: 95), IFN-γ-AAAGAGATAATCTGGCTCTGC (SEQ ID NO: 96) and GCTCTGAGACAATGAACGCT (SEQ ID NO: 97), CCL5-TCCTTCGAGTGACAAACACG (SEQ ID NO: 98) and CCCTCACCATCATCCTCACT (SEQ ID NO: 99), CXCL9-TGAGGTCTTTGAGGGATTTGTAGTG (SEQ ID NO: 100) and GGAACCCTAGTGATAAGGAATGCA (SEQ ID NO: 101), CXCL10-GACGGTCCGCTGCAACTG (SEQ ID NO: 102) and CTTCCCTATGGCCCTCATTCT (SEQ ID NO: 103), PD-L1-GACCAGCTTTTGAAGGGAAATG (SEQ ID NO: 104) and CTGGTTGATTTTGCGGTATGG (SEQ ID NO: 105), and GAPDH-TGACCTCAACTACATGGTCTACA (SEQ ID NO: 106) and CTTCCCATTCTCGGCCTTG (SEQ ID NO: 17). Results from quantitative (q)RT-PCR analysis showed that FBXO44 KD activated the transcription of various RE subfamilies (FIG. 1P and FIG. 2B), including REs that displayed decreased H3K9me3 level in FBXO44 KD cells, indicating they were direct targets of FBXO44. RNA-seq confirmed the upregulated expression of various REs in FBXO44 KD cells (FIG. 2C). Of note, the RE subtypes activated by FBXO44 KD partially overlapped with those activated by targeting Histone H3K4 demethylase, LSD1 (FIG. 2D). Together, these findings demonstrate that FBXO44 co-localized with H3K9me3 modifications and was essential for RE transcriptional silencing in cancer cells.

### Example 5: Identification of FBXO44 and Associated Proteins on REs

Protein mass spectrometry was performed to investigate FBXO44 and associated proteins on chromatin.

Mass spectrometry: for analysis of FBXO44 in WCE, Flag-FBXO44 was introduced into Flp-In TREx-HeLa cells (ThermoFisher Scientific no. R78007) and FBXO44 protein complexes affinity purified. For analysis of FBXO44 in chromatin fractions, MDA-MB-231 (control) or MDA-MB-231-Flag-FBXO44 cells were enriched in S phase by double thymidine block (two sequential 2 mM overnight incubations) and released into normal medium for 3 hr and harvested, washed 2× with cold PBS, and chromatin isolated. Chromatin was dissolved in 2 mL IP buffer and sonicated until the solution cleared. After centrifugation at 12,000×g for 20 min at 4° C., the supernatants were collected and adjusted to 3 mL with IP buffer and pre-cleared by incubation with 10 μL of normal rabbit IgG and 60 μL of protein G agarose beads for 2 hr at 4° C. The supernatants were then incubated with 12.5 μL of anti-Flag antibody (Sigma-Aldrich no. F7425) overnight at 4° C. After 3×10 min washes with IP buffer, the immunoprecipitates were eluted 3× with 150 μL of Flag peptide elution buffer (Sigma-Aldrich no. FLAGIPT1). Trichloroacetic acid (Sigma-Aldrich no. T0699) was then added to the eluents to a final concentration of 20% and incubated on ice for 1 hr. After centrifugation at 14,000×g for 25 min at 4° C., pellets were washed with 500 μl of ice-cold acetone. After another centrifugation at 14,000×g for 25 min at 4° C., the pellets were air-dried in a fume hood for 30 min and stored at −20° C. Mass spectrometry analysis was performed using duplicate samples.

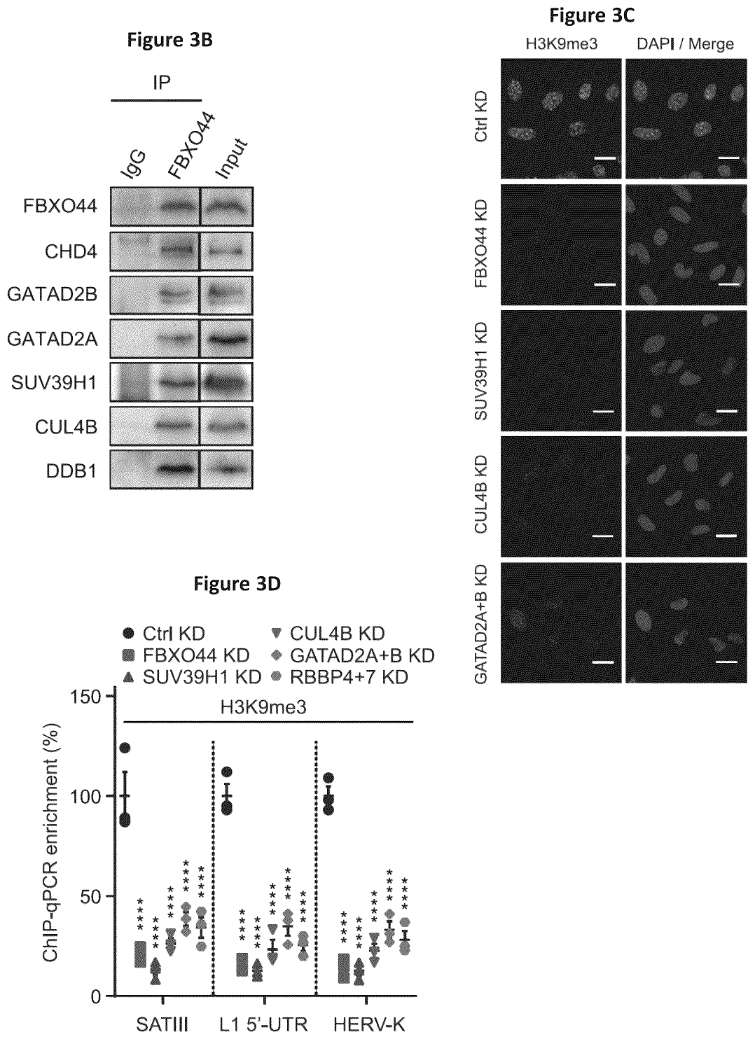

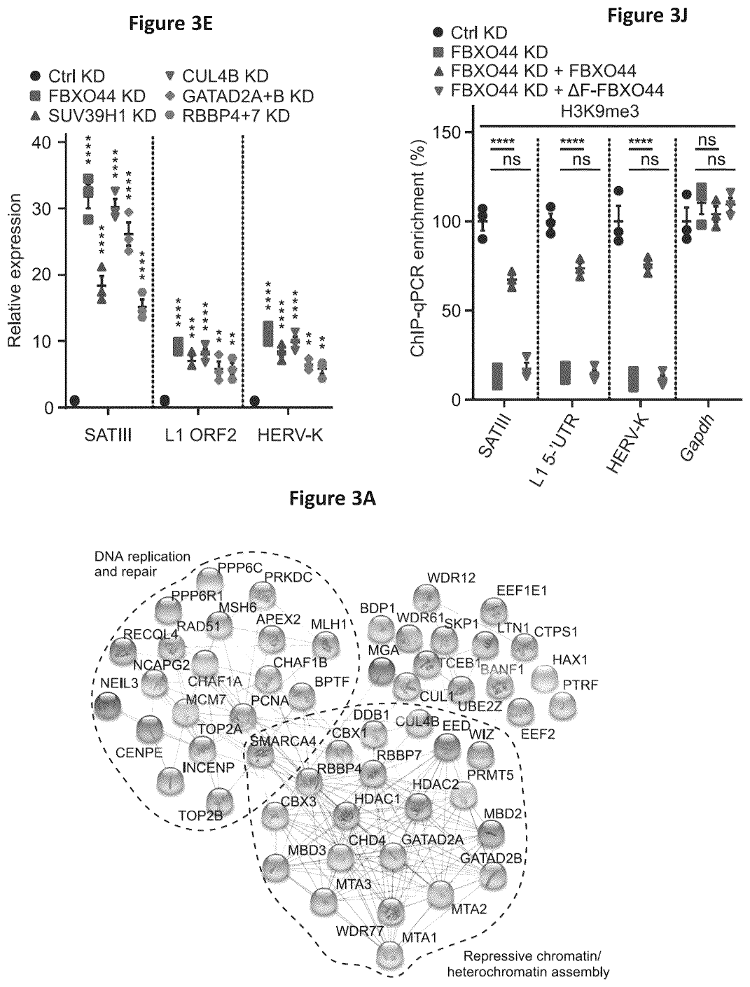

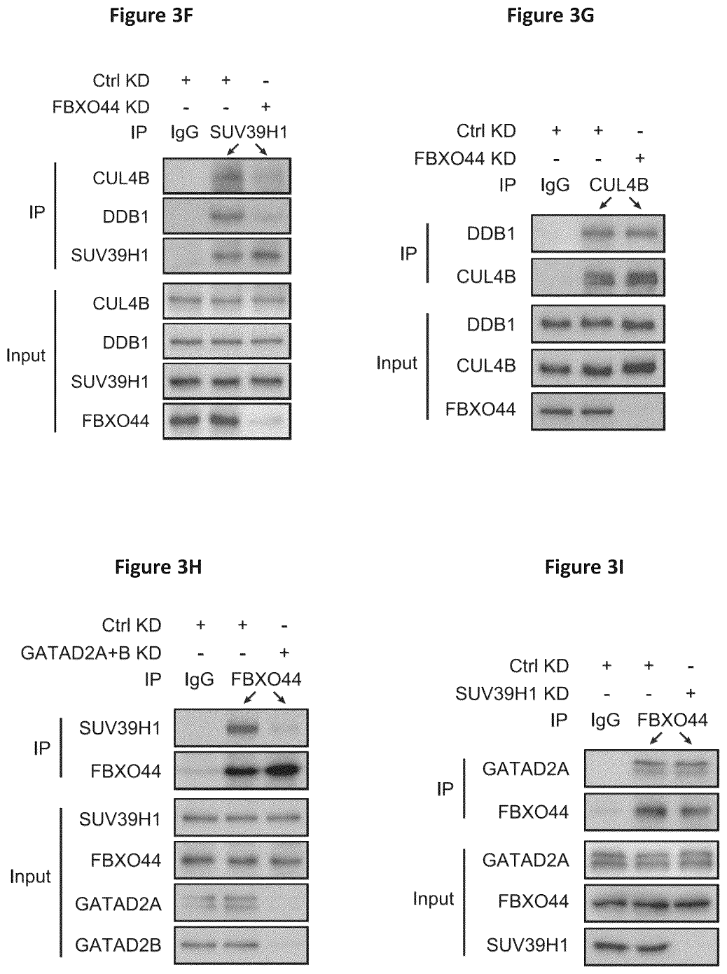

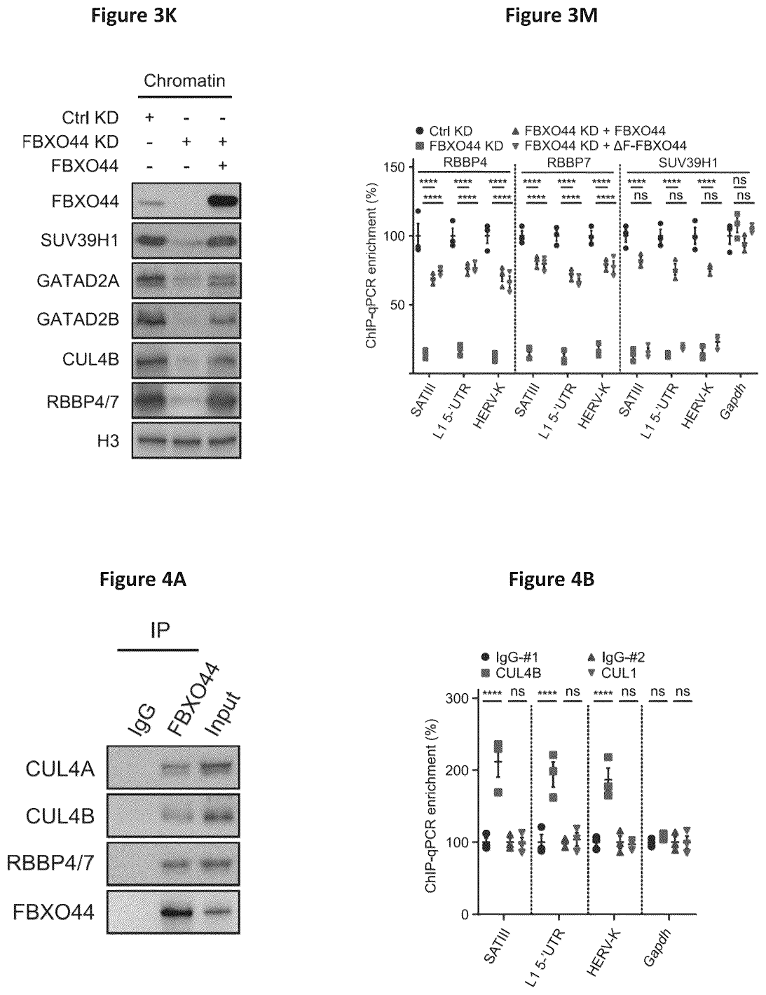

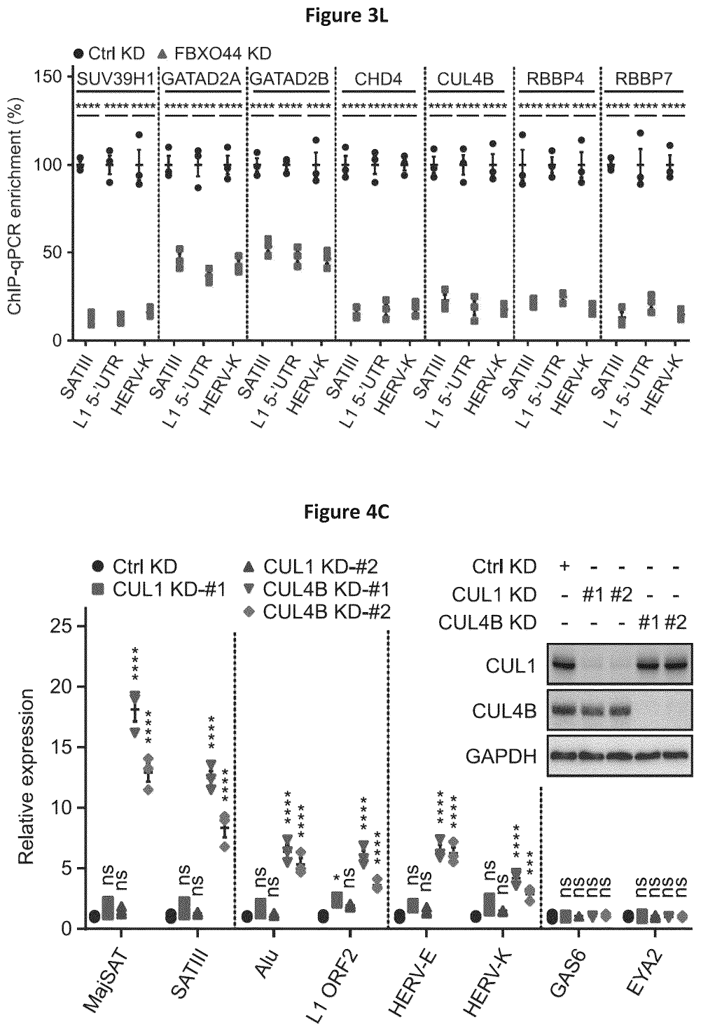

Protein mass spectrometry analysis revealed that FBXO44 interacted with several chromatin modifiers/remodelers previously implicated in heterochromatin assembly (FIG. 3A), including components of Mi-2/NuRD (GATAD2A/B, CHD4, MBD2, MTA1-3, and HDAC1-2), CTBP transcriptional co-repressor (PRMTS, MEP50, WIZ), and polycomb repressor complex (PRC2)/EED-EZH2 (EED). FBXO44 also interacted with the CRL4 ubiquitin ligase (DDB1 and CUL4B), as well as DDB1 and CUL4-associated factors (DCAFs) RBBP4 and RBBP7, which was implicated in regulation of H3K9me3 and H3K27me3 in human cells.

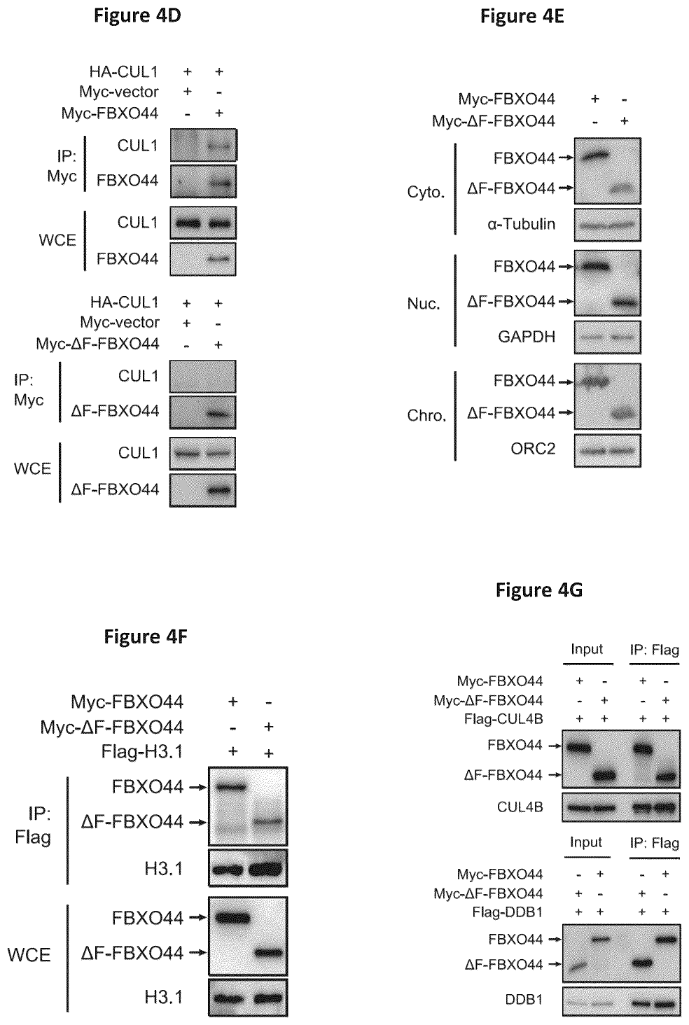

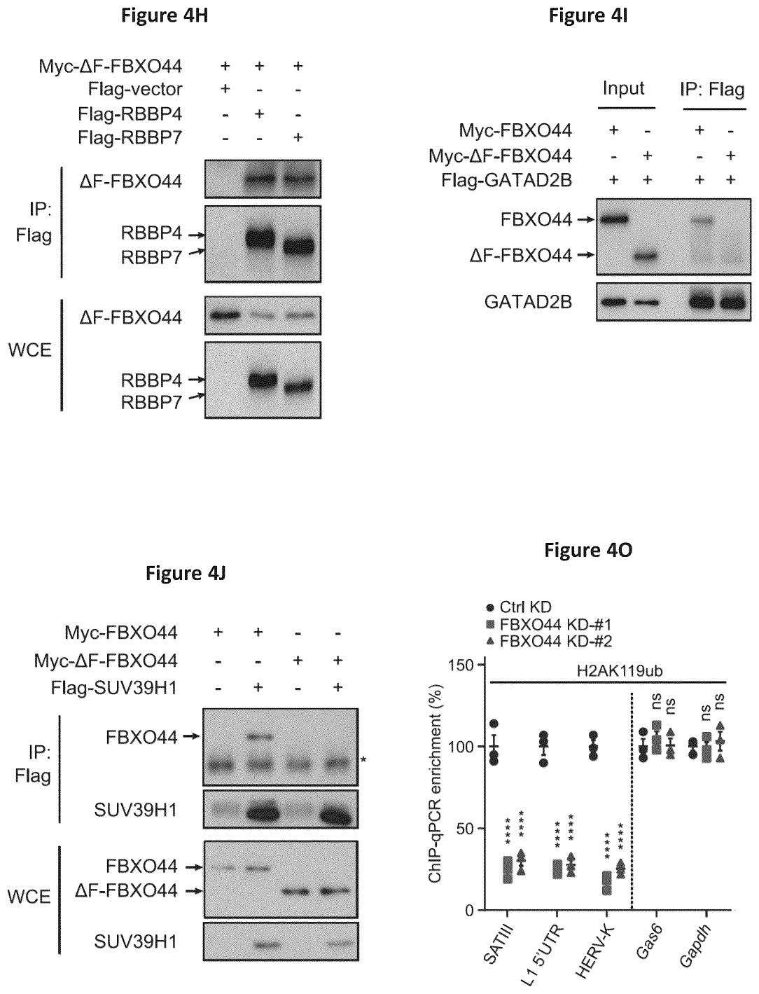

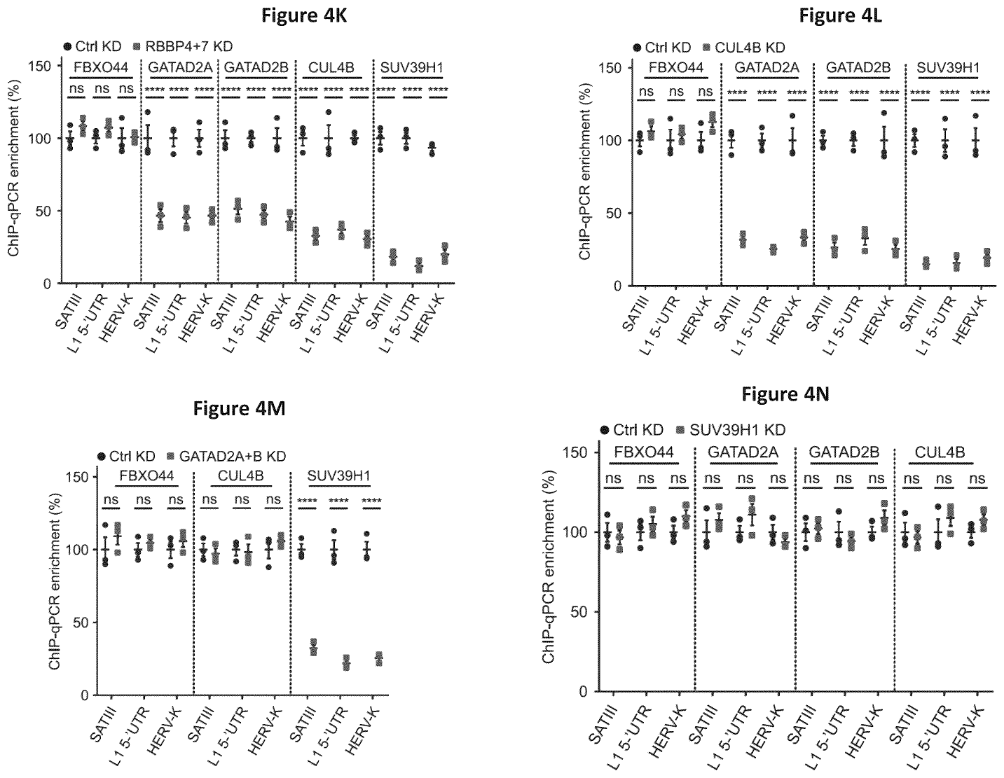

To confirm mass spectrometry results, co-IP was performed using method described in example 3. Co-IP result confirmed FBXO44 interacted with components of Mi-2/NuRD and CRL4, as well as RBBP4/7 (FIGS. 3B and 4A). Moreover, FBXO44 interacted with H3K9me3 methyltransferase SUV39H1 (FIG. 3B).

To determine if FBXO44 cooperated with SUV39H1, CRL4RBBP4/7 and Mi-2/NuRD in RE silencing, each enzyme/complex component was targeted, and H3K9me3 and RE transcription levels were analysed. Methods for siRNA KD, IF, ChIP, and qRT-PCR were described in previous examples. Targeting SUV39H1 or CUL4B by KD, or co-KD of Mi-2/NuRD components GATAD2A+B, decreased total chromatin associated H3K9me3 modifications, comparable to FBXO44 KD (FIG. 3C). ChIP experiments showed targeting SUV39H1, CUL4B, GATAD2A+B, or RBBP4+7 decreased H3K9me3 modifications at various REs, similar to FBXO44 KD (FIG. 3D). These KD cells also activated RE transcription comparable to FBXO44 KD cells (FIG. 3E). Although IP experiments demonstrated that FBXO44 interacted with CUL1 in chromatin fractions (FIG. 1F) ChIP experiments showed CUL4B, but not CUL1, was enriched at REs (FIG. 4B). Further, CUL1 KD did not activate RE transcription (FIG. 4C). Collectively, these results suggest FBXO44 serves a CUL1-independent function by cooperating with SUV39H1, CRL4RBBP4/7, and Mi-2/NuRD to transcriptionally silence REs in cancer cells.

Next, to characterize FBXO44's molecular interactions with interactions with SUV39H1, CRL4RBBP4/7, and Mi-2/NuRD, Co-IP experiments were performed as described in example 3. For SUV39H1 IP, anti-SUV39H1 antibody (Millipore no. 05-615) was used at 1:100 dilution. For CUL4B IP, 2 μg of anti-CUL4B antibody (Proteintech no. 12916-1-AP) was used for 2500 μg of protein lysate. Results showed that showed that endogenous SUV39H1 interacted with CRL4 components, CUL4B and DDB1, and these interactions were FBXO44-dependent (FIG. 3F). However, FBXO44 KD did not affect CRL4 complex assembly (FIG. 3G). Interaction of FBXO44 and SUV39H1 depended on Mi-2/NuRD components GATAD2A+B, although interaction between FBXO44 and GATAD2B was SUV39H1-independent (FIGS. 3H-3I).

### Example 6: Identification of FBXO44 Functions as an Adaptor Protein, Acting Upstream of FBXO44-Associated Proteins in RE Silencing

FBXO44 consists of an N-terminal F-box domain and C-terminal F-box-associated domain (FBA), which presumably binds substrates. To investigate function of FBXO44, siRNA-mediated KD, coIP, immunoblotting, and ChIP experiments were performed as described in previous examples. Results from ChIP and immunoblotting experiments showed that expression of an siRNA-resistant cDNA encoding F-box deleted FBXO44 (AF-FBXO44) that did not interact with CUL1 but appropriately localized in cells (FIGS. 4D-4E), could not compensate for FBXO44 in mediating H3K9me3 modifications at REs in FBXO44 KD cells (FIG. 3J). Co-IP experiments revealed that AF-FBXO44 interacted with Histone H3.1, CUL4B/DDB1, and RBBP4/7, but not GATAD2B or SUV39H1 (FIGS. 4F-4J). Together, these data suggest that FBXO44 functions as an adaptor protein, with its FBA domain mediating interactions with chromatin and CRL4RBBP4/7 and N-terminal F-box-containing region required for interaction with SUV39H1 and Mi-2/NuRD.

To investigate recruitment of FBXO44 and its associated proteins, siRNA-mediated KD and ChIP experiments were performed as described in previous examples. Results showed that as FBXO44 targeting decreased the interaction of SUV39H1, CUL4B, and Mi-2/NuRD with chromatin, a phenotype rescued by expression of an siRNA-resistant FBXO44 cDNA (FIG. 3K), FBXO44 might recruit these enzymes to REs to initiate transcriptional silencing. Results from ChIP experiments showed that FBXO44 KD prevented recruitment of SUV39H1, CUL4B, RBBP4/7, and Mi-2/NuRD (GATAD2A/B and CHD4) to REs (FIG. 3L). Targeting RBBP4+7 inhibited recruitment of SUV39H1, CUL4B, and GATAD2A/B, but not FBXO44 (FIG. S2K). CUL4B reduction prevented SUV39H1 and GATAD2A/B recruitment, although FBXO44 was unaffected (FIG. 4L). Further, targeting GATAD2A+B inhibited SUV39H1 recruitment, but not FBXO44 or CUL4B (FIG. 4M). In contrast, SUV39H1 KD had no effect on FBXO44, CUL4B, RBBP4/7, or GATAD2A/B recruitment to REs (FIG. 4N). These data suggest that FBXO44 functions upstream of SUV39H1, CRL4RBBP4/7, and Mi-2/NuRD in RE silencing. In support of this, introduction of AF-FBXO44 into FBXO44 KD cells promoted recruitment of RBBP4/7, but not SUV39H1, to REs (FIG. 3M). ChIP experiments also demonstrated that FBXO44 KD diminished mono-ubiquitylation of H2AK119 at REs (FIG. 4O), a repressive chromatin modification mediated, in part, by CRL4.

### Example 7: Identification of FBXO4 in Cell Cycle

Mass spectrometry was performed as described in previous examples and results revealed that FBXO44 interacted with several DNA replication proteins, including PCNA, CHAF1A/B, and MCMI (FIG. 3A). In order to investigate the relationship of FBXO44 and cell cycle synchronization, thymidine block, IF, co-IP experiments were performed as described in previous example. In the thymidine block IF method, inhibitors of DNA synthesis including thymidine, aphidicolin, and hydroxyurea are used to trap cells in S phase. High concentration of thymidine interrupts the deoxynucleotide metabolism pathway, thereby halting DNA replication. As treatment with thymidine arrests cells throughout S phase, a double thymidine block procedure (which involves releasing cells from a first thymidine block before trapping them with a second thymidine block) is generally used to induce a more synchronized early S phase blockade.

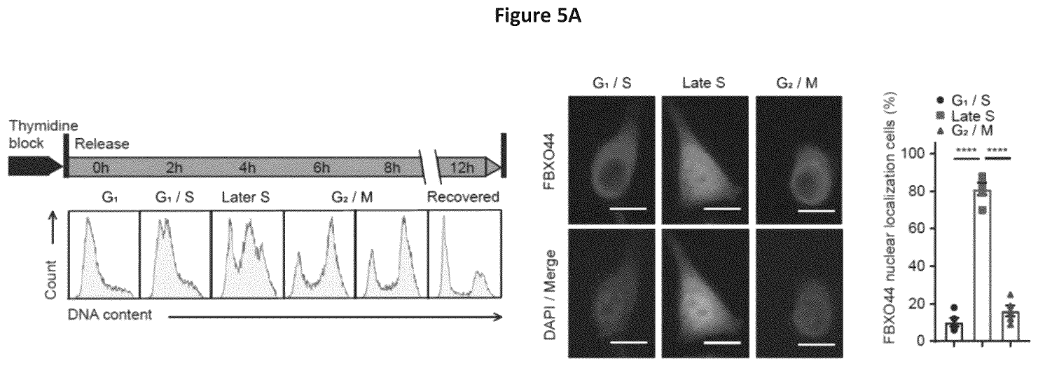

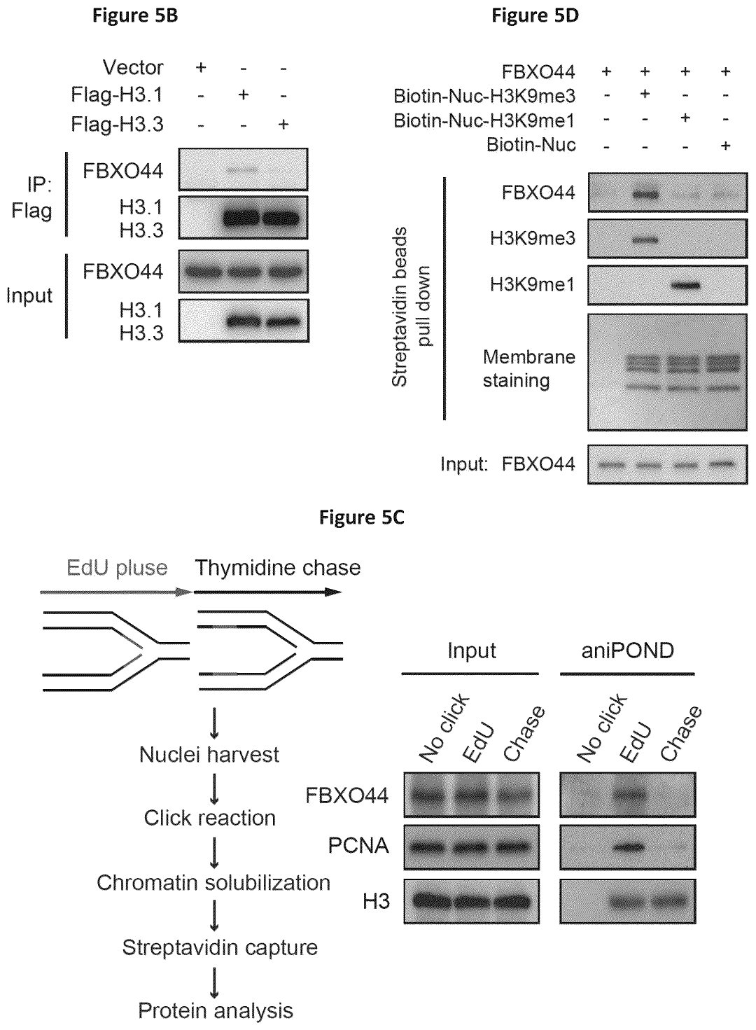

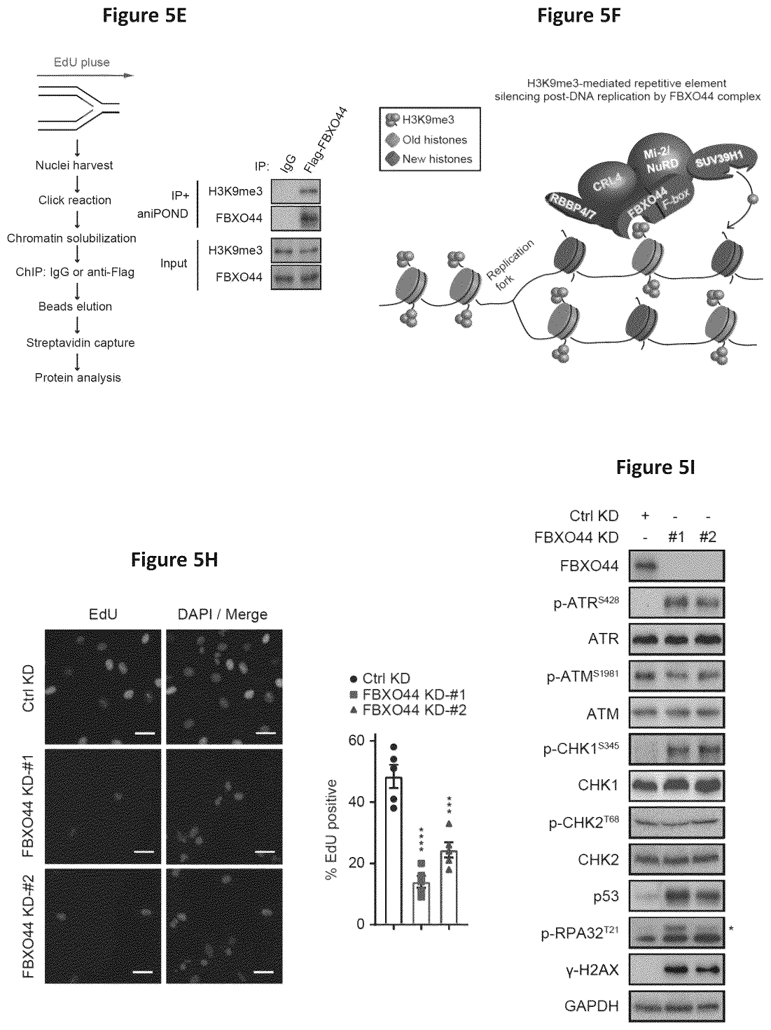

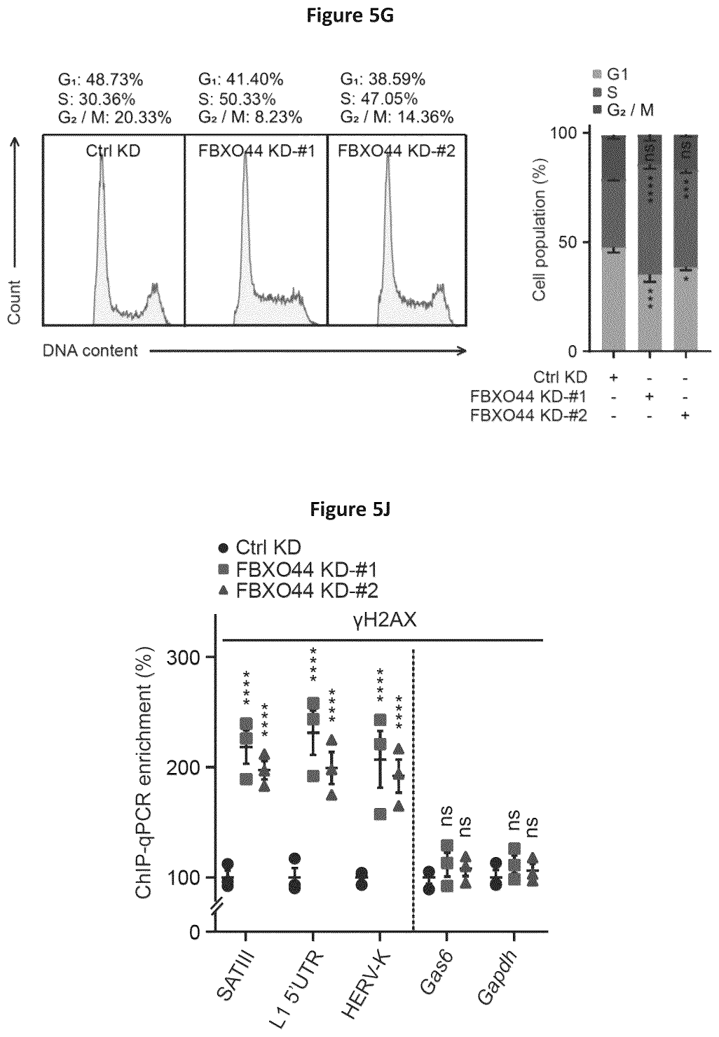

IF results showed that FBXO44 accumulated in the nucleus in S phase (FIG. 5A). Co-IP experiments also showed that FBXO44 preferentially interacted with DNA replication-associated Histone H3.1, relative to Histone H3.3 (FIG. 5B), which is deposited by a DNA synthesis-independent pathway.

### Example 8: Identification of FBXO44 and Chromatin Binding

To investigate FBXO44's interaction with chromatin as a function of DNA replication, accelerated native isolation of protein on nascent DNA (aniPOND) analysis with modifications and immunoblotting were performed. Immunoblotting was performed as described in previous examples.

aniPOND analysis: briefly, MDA-MB-231 cells (˜8.5×10⁷) were cultured in medium containing 10 μM EdU for 30 min and then harvested or chased with medium containing 10 μM thymidine for 60 min and harvested. Nuclei were isolated by addition of nuclear extraction buffer. Biotin-azide click reactions were performed on a rotator for 1 hr at 4° C., and 50 μL of streptavidin beads (Cell Signaling Technology no. 3419) were used for streptavidin capture for each sample. Beads were washed 3×10 min and boiled in 1×SDS gel loading buffer prior to electrophoresis. For aniPOND analysis of FBXO44 binding to H3K9me3-modified nucleosomes, MDA-MB-231-Flag-FBXO44 cells (˜1.7×10⁸) were incubated with 10 μM EdU for 30 min, harvested, subjected to biotin-azide click reactions, and chromatin sonicated prior to IP with IgG or anti-Flag antibody (Sigma-Aldrich no. F3165). Chromatin was eluted 3× with Flag peptide solution (150 ng/μL) and subjected to affinity purification with streptavidin beads.

Results showed that FBXO44 specifically interacted with newly replicated chromatin and dissociated within 60 min following DNA replication (FIG. 5C).

Next, to identify the potential trigger for FBXO44 chromatin binding, in vitro binding assays were performed.

In vitro binding assays: synthetic biotinylated nucleosomes (EpiCypher no. 16-0006, 16-0315, 16-0325; 25 μg) were incubated with 10 μL of streptavidin beads (Cell Signaling Technology no. 3419) in binding buffer (50 mM Tris-HCl [pH 7.4], 125 mM NaCl, 1 mM EDTA, 0.1% Triton X-100, 50 mM NaF, 1 mM PMSF, 1 mM Na₃VO₄, 1 μg/mL Aprotinin, 1 μg/mL Leupeptin, 1 μg/mL Pepstatin) on a rotator for 30 min at 4° C. Then, 8 μg of recombinant FBXO44 protein (OriGene no. TP760409) was added and incubated for 2 hr at 4° C. in 250 total volume. Freeze-thaw cycles were avoided with recombinant proteins. After centrifugation, the beads were washed 3×10 min with binding buffer and boiled in 1×SDS gel loading buffer prior to electrophoresis.

Results from in vitro binding assays using recombinant FBXO44 and synthetic H3K9me3-, H3K9me1-, or un-modified nucleosomes revealed that FBXO44 selectively bound to H3K9me3-modified nucleosomes (FIG. 5D), consistent with ChIP-seq data that showed FBXO44 co-localized with H3K9me3 modifications in cells (FIGS. 1J-1M and 2A). Results from a modified aniPOND experiment demonstrated that FBXO44 bound H3K9me3-modified nucleosomes of newly replicated chromatin (FIG. 5E). All together, these results demonstrate that FBXO44 bound H3K9me3-modified nucleosomes at the replication fork to initiate RE silencing post-DNA replication (FIG. 5F).

### Example 9: Identification of FBXO44/SUV39H1 Inhibition and Relationship to DNA Replication Stress and DNA Double-Strand Breaks (DSBs)

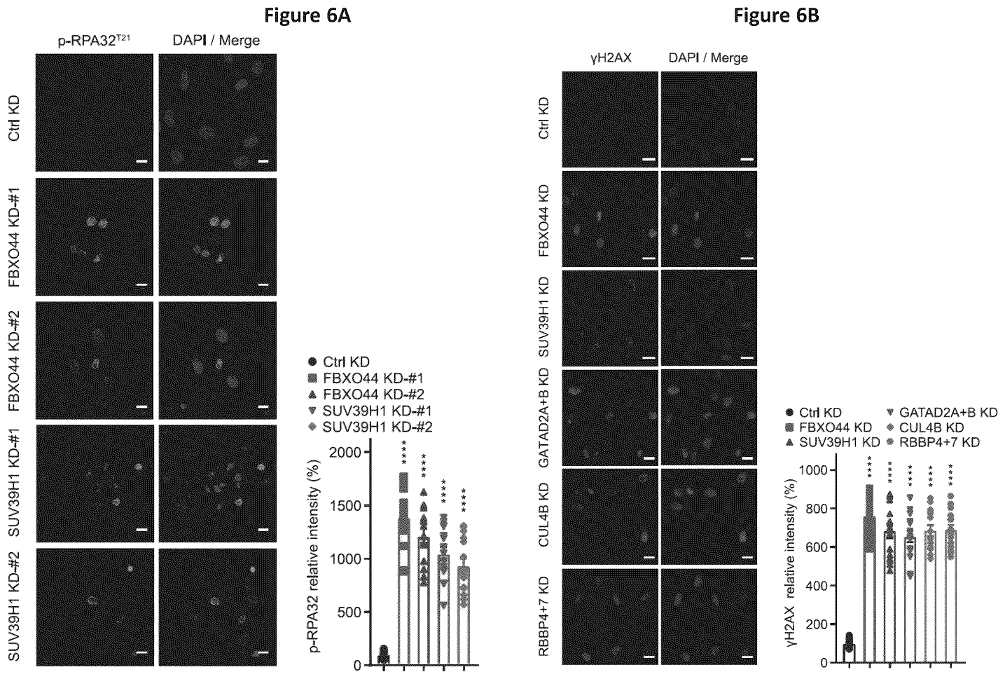

To investigate level of p-RPA32^{T21}, a marker for DNA replication stress, IF was performed as described in previous examples. The relevant primary antibodies diluted in blocking buffer were then added. Specifically, 1:2000 dilution for anti-p-RPA32^{T21} (GeneTex no. GTX62664) was used. Results showed that FBXO44/SUV39H1 KD increased the level of p-RPA32^{T21} in cancer cells, indicating DNA replication stress (FIG. 6A).

Flow cytometry, IF, and immunoblotting were performed to analyse cell cycle. Samples were analyzed using an LSR Fortessa instrument (BD PharMingen) and data analyzed with FlowJo Software (Treestar). Results showed that FBXO44 KD cells also accumulated in S phase and displayed reduced EdU incorporation (FIGS. 5G-5H). Furthermore, immunoblot was performed as described in previous examples and results showed that FBXO44 KD activated the DNA replication checkpoint, as indicated by increased p-ATR^{S428} and p-CHK1^{S345} (FIG. 5I). Targeting FBXO44/SUV39H1, GATAD2A+B, CUL4B, or RBBP4+7, increased γH2AX, indicating DNA double-strand breaks (DSBs) (FIGS. 51 and 6B), and activated p53 expression (FIG. 5I). ChIP was also performed as described in previous examples and results showed that FBXO44 KD induced γH2AX was enriched at REs compared to randomly selected control genes (FIG. 5J). Thus, FBXO44/SUV39H1 inhibition promotes DNA replication stress and DSBs in cancer cells.

### Example 10: Identification of FBXO44 and Interaction with RNA and DNA from REs

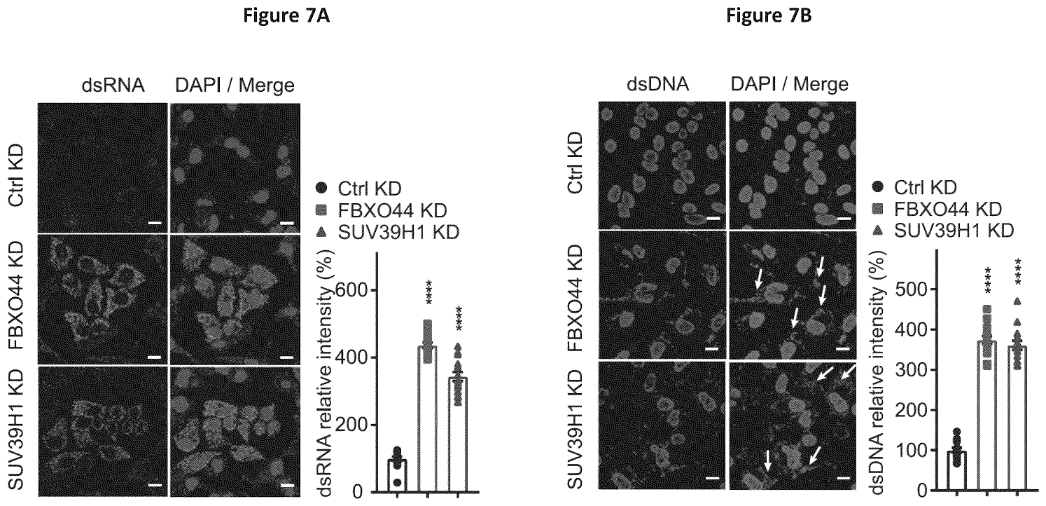

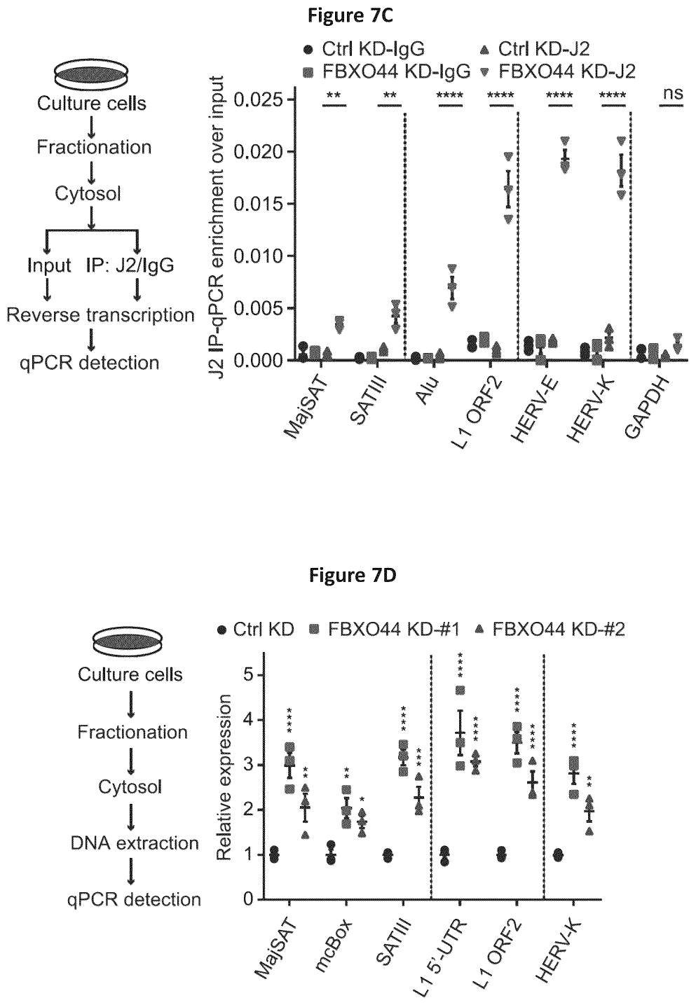

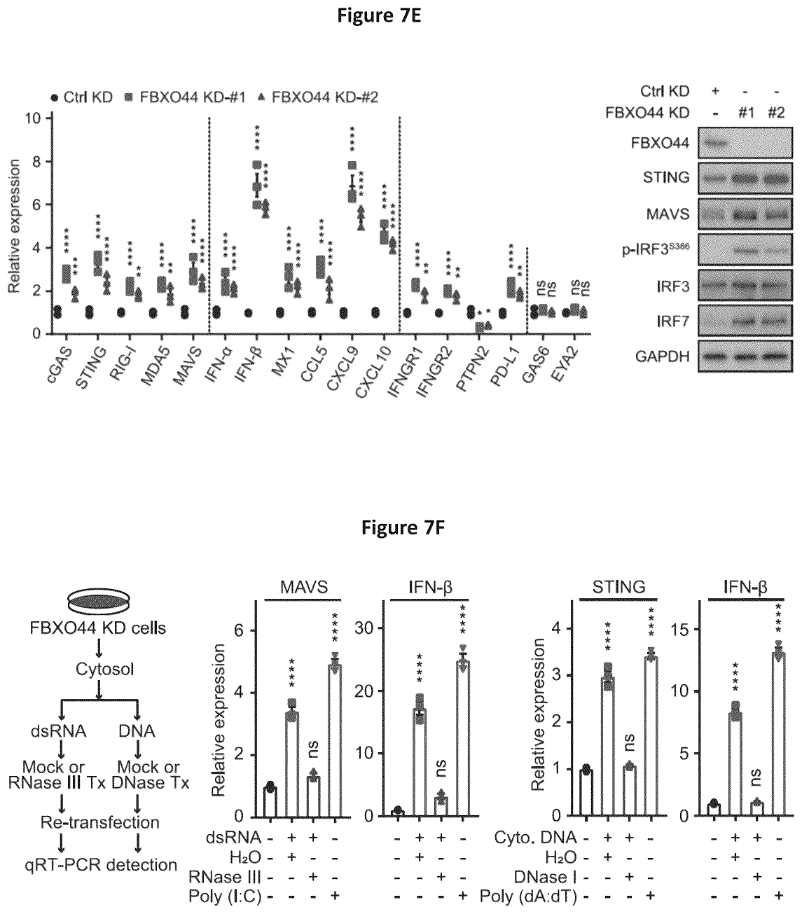

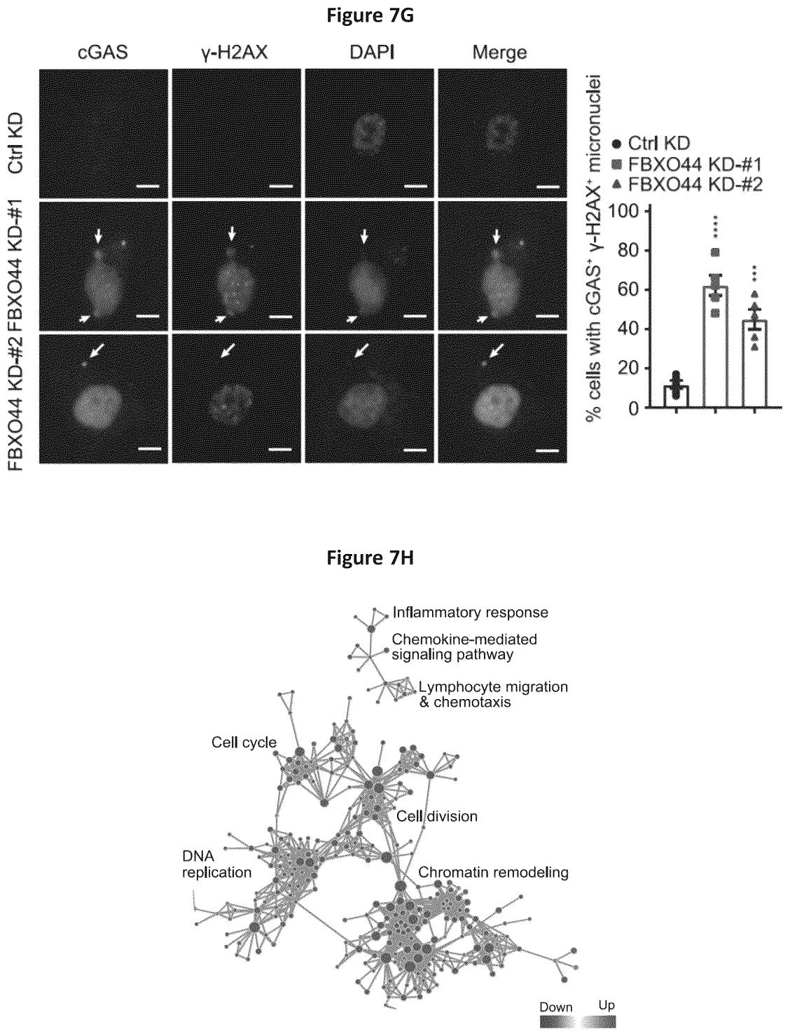

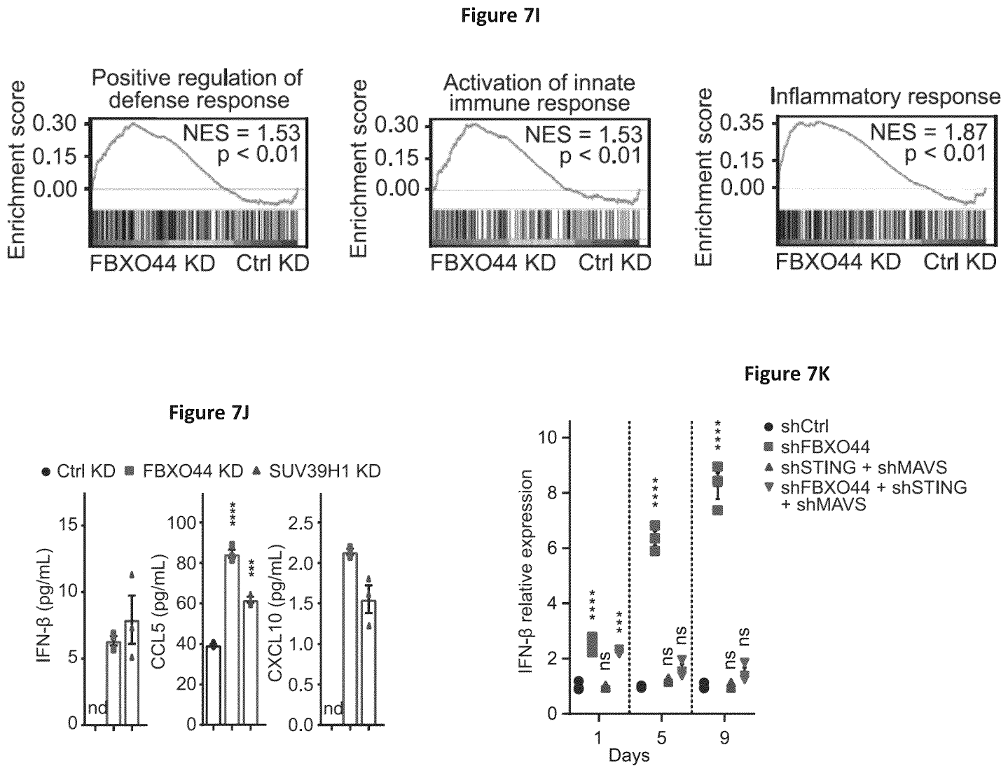

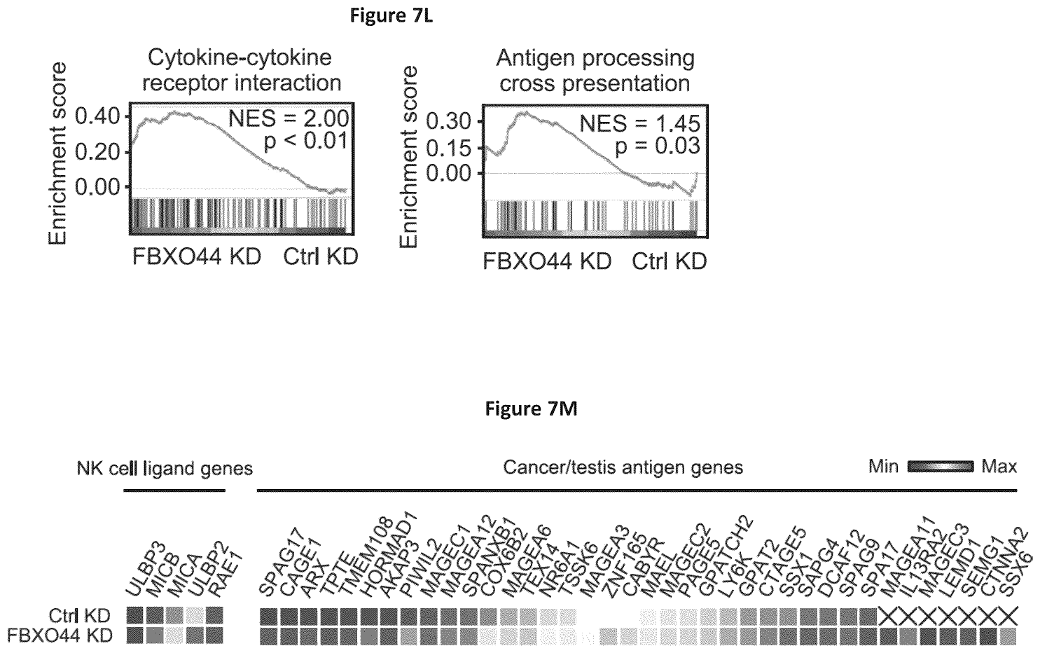

To investigate dsRNA and dsRNA in relation to FBXO44/SUV39H1, IF was performed as described in previous examples. The relevant primary antibodies diluted in blocking buffer were then added. Specifically, 1:60 dilution for anti-dsRNA (Millipore no. MABE1134) or 1:1000 dilution for anti-dsDNA (Abcam no. ab27156) were used. IF results showed that FBXO44/SUV39H1 targeting induced the accumulation of cytosolic dsRNA and dsDNA in cancer cells (FIGS. 7A-7B).

Immunoprecipitation (IP) of the cytosolic dsRNA and RNase A protection experiments were performed to investigate FBXO44 and interaction with RNA and DNA. KD, IP, ChIP, and q-PCR experiments were performed as described in previous examples.

Cytoplasmic DNA isolation and analyses: equal amounts of MDA-MB-231 cells (5×106) transfected with control or 2 different FBXO44 siRNAs were harvested and washed with cold PBS. Cytoplasmic fractions were extracted from the cells after the nuclear fraction was removed through centrifugation using the Nuclear Extract Kit (Active Motif no. 40010) according to manufacturer's instructions. Cytoplasmic fraction (390 μL solution) was combined with 25 μL 5M NaCl, then treated with 8 μL RNase A/T1 Mix (Thermo Fisher Scientific no. EN0551) for 30 min at 37° C. prior to DNA extraction using the Gel Extraction Kit (Qiagen no. 28704). The amount of indicated RE DNA in cytosol were determined using qPCR primers in ChIP analysis and normalized based on gapdh level.

Cytoplasmic dsRNA isolation and analyses: control or FBXO44 KD MBA-MB-231 cells (4.5×10⁶) were harvested and cytoplasmic fractions extracted using the Nuclear Extract Kit (Active Motif no. 40010) according to the manufacturer's instructions. An equal volume of 70% ethanol (350 μL) was added to the cytoplasmic fractions and then RNA isolated using the RNeasy Plus Mini Kit (Qiagen no. 74134). The total RNA was dissolved with 38 μL RNase-free H₂O. Then 2 μL total RNA was used as input and the remainder divided into 2 tubes (18 μL in each). J2 antibody (Scicons no. 10010200) and normal mouse IgG (Santa Cruz no. sc-2025) were conjugated (2 μg per pulldown) to 20 μL protein G agarose (Millipore no. 16-266) by rotation for 2 hr at 4° C. To each tube containing total RNA, 1 μL of RNase A (10 mg/mL, Sigma-Aldrich no. R6513) was added and then mixed with 1 mL IP buffer (50 mM Tris-HCl [pH 7.4], 125 mM NaCl, 1 mM EDTA, 0.1% Triton X-100), followed by incubation with conjugated protein G agarose beads for 2 hr at 4° C. Beads were washed 3× with IP buffer and incubated in 50 μL proteinase K digestion solution (1×TE, 100 mM NaCl, 1% SDS, and 1 μL of 20 mg/mL Proteinase K solution (Thermo Fisher Scientific no. AM2546)) for 20 min at 45° C. After centrifugation, the eluate (50 μL) was added to 300 μL Buffer RLT Plus from the RNeasy Plus Mini Kit (Qiagen no. 74134) and RNA isolated. The final product containing dsRNA was denatured at 95° C. for 5 min, followed by reverse transcription and qPCR reactions using the primers in gene expression analysis.

RNAase protection assay: RNA protection assay was performed as described previously with modifications. Total RNA from MDA-MB-231 cells (2×10⁶) transfected with control or 2 different FBXO44 siRNAs was isolated using the RNeasy Plus Mini Kit (Qiagen no. 74134) and 4 μg of total RNA dissolved in 49.5 μL RNase protection buffer (10 mM Tris-HCl, pH 7.5, 350 mM NaCl). Then 0.5 μL RNase A (10 mg/mL, Sigma-Aldrich no. R6513) was added and the mixture incubated for 30 min at 37° C. dH₂O was added to the control sample. dsRNA was isolated and denatured at 95° C. for 5 min. Reverse transcription was carried out using qScript cDNA SuperMix (Quantabio no. 95048) and qPCR reactions performed using primers listed in the “qPCR analyses” section. dsRNA enrichment for selected REs was calculated by (RE/GAPDH) RNase A/(RE/GAPDH) control.

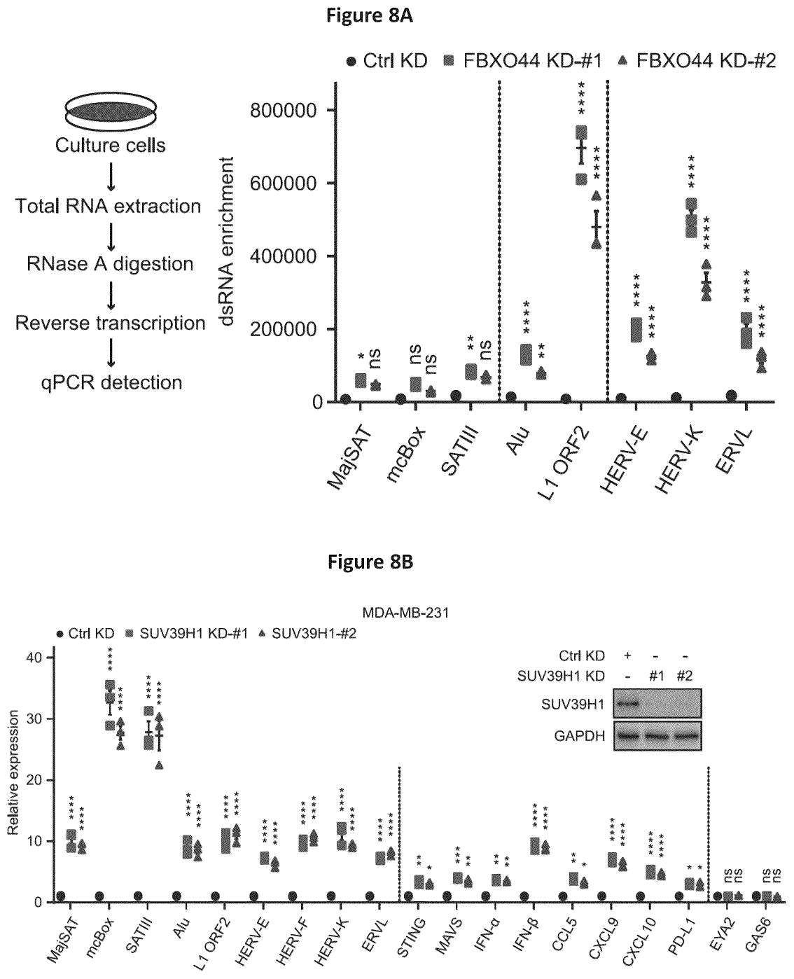

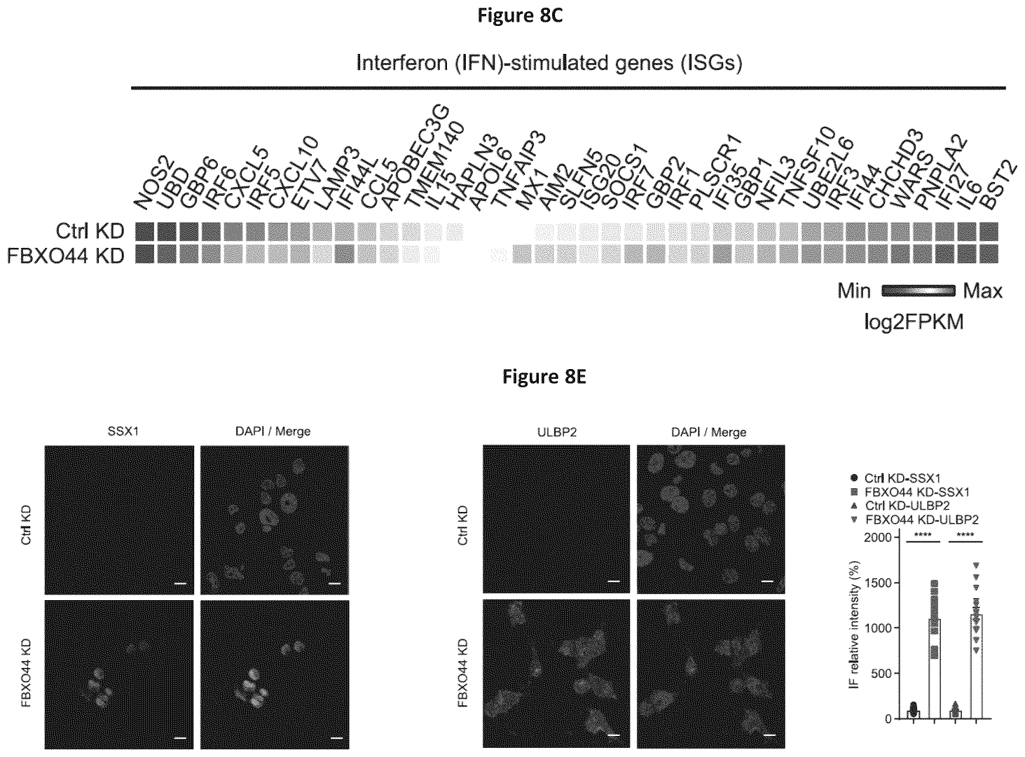

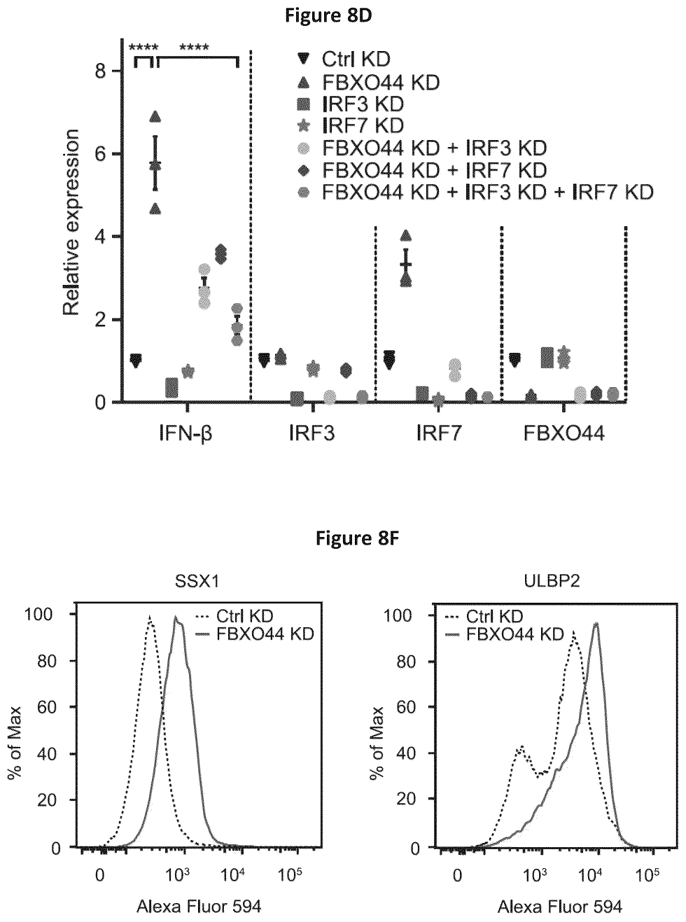

Results showed that FBXO44 KD cells contained dsRNA generated from various REs subtypes (FIGS. 7C and 8A). FBXO44 KD cells also contained cytosolic dsDNA generated from these RE subtypes (FIG. 7D).

### Example 11: Identification of FBXO44 and Activation of Antiviral Pathway

To investigate the interaction of FBXO44 and RIG-I/MDA5-MAVS and cGAS-STING antiviral pathways, KD, qRT-PCR, immunoblotting experiments were performed as described previously.

Results showed that RIG-I/MDA5-MAVS and cGAS-STING antiviral pathways were activated in FBXO44/SUV39H1 KD cells (FIGS. 7E, 2B, and 8B). FBXO44 KD cells also displayed increased p-IRF3S386, a modification that stimulates IRF3 nuclear translocation and activity, and increased IRF7 expression (FIG. 7E).

To investigate the activation of MAVS or STING transcription, transfection experiment of cytosolic dsRNA or dsDNA isolated from FBXO44 KD cells was performed. qRT-PCR and IF experiments were performed as described in previous examples.

Cytoplasmic dsRNA/DNA re-transfection: transfection of cytosolic dsRNA or dsDNA isolated from FBXO44 KD cells was performed. Briefly, preparation of cytoplasmic fractions of cultured cells was performed and cytoplasmic DNA extracted using the Gel Extraction Kit (Qiagen no. 28704) after incubation with RNase A/T1 Mix (Thermo Fisher Scientific no. EN0551) as detailed in the “Cytoplasmic DNA isolation and qPCR analyses” section. For dsRNA extraction, the cytoplasmic fraction was used for total RNA isolation using the RNeasy Plus Mini Kit (Qiagen no. 74134) and total RNA digested with RNase A solution to preserve dsRNA as detailed in the “RNase protection assay” section. The concentrations of cytoplasmic dsRNA and DNA were measured using a Nanodrop 2000 spectrophotometer (Thermo Fisher Scientific). For the cytoplasmic DNA re-transfection assay, 1 μg cytoplasmic DNA was digested with 1 μL DNase I (Thermo Fisher Scientific no. EN0521) or 1 μL H₂O (mock digestion) in 10 μL reaction mixture for 30 min at 37° C. prior to re-transfection into MDA-MB-231 cells with 1.5 μL Lipofectamine 2000 (Thermo Fisher Scientific). Transfection of 0.8 μg poly (dA:dT) (InvivoGen no. tlrl-patn) was used as a positive control. For cytoplasmic dsRNA re-transfection, μg of dsRNA was digested with 1 μL RNase III (Thermo Fisher Scientific no. AM2290) or 1 μL H₂O (mock digestion) in 5 μL reaction mixture for 60 min at 37° C. prior to re-transfection into MDA-MB-231 cells with 1.5 μL Lipofectamine 2000. Transfection of 0.8 μg poly (I:C) (InvivoGen no. tlrl-pic) was used as positive control. After 72 hr, RNA was isolated and qPCR reactions performed using primers in gene expression analysis.

Results showed that transfection of cytosolic dsRNA or dsDNA isolated from FBXO44 KD cells activated MAVS or STING transcription, respectively, in recipient cells (FIG. 7F). IF was also performed to investigate the level of cGAS and γH2AX, and results showed that FBXO44 KD cells also exhibited increased micronuclei that stained positive for cGAS and γH2AX compared to control cells (FIG. 7G), consistent with their generation via genomic instability. Together, these data suggest that cytosolic dsRNA and dsDNA generated from REs, as well as DNA replication stress, contribute to activate antiviral pathways in FBXO44 KD cells.

### Example 12: Identification of FBXO44 and Regulation of Defense Response, Innate Immune Response, and Inflammatory Response

RNA-seq was performed to investigate gene set enrichment due to FBXO44 KD. RNA-seq: polyA+ RNA was isolated using the NEBNext Poly(A) mRNA Magnetic Isolation Module and barcoded libraries made using the NEBNext Ultra II Directional RNA Library Prep Kit for Illumina (NEB). Libraries were pooled and single end sequenced (1×75) on an Illumina NextSeq 500 using the High Output V2 Kit (Illumina). Read data was processed in BaseSpace (basespace.illumina.com). Reads were then aligned to the Homo sapiens genome (hg19) using STAR aligner (https://code.google.com/p/rna-star/) with default settings. Differential transcript expression was determined using the Cufflinks Cuffdiff package (https://github.com/cole-trapnell-lab/cufflinks). GSEA was performed on pre-ranked gene lists upon FBXO44 KD using gene set permutation for statistical testing. GSEA results were visualized using Enrichment Map in Cytoscape. For analysis of RE expression from RNA-seq data, reads were mapped to the Homo sapiens genome (hg19) using Bowtie 2 and assigned to REs using RepEnrich2 with the recommended parameters (https://github.com/nerettilab/RepEnrich2). The RE annotation file (hg19_repeatmasker_clean.txt) was provided by RepEnrich2. The resulting counts for REs were analyzed by the edgeR package to obtain CPM (counts per million reads) values.

Gene set enrichment analysis (GSEA) of FBXO44 KD RNA-seq data was performed, and results revealed positive regulation of defense response, activation of innate immune response, and inflammatory response among the most upregulated gene expression pathways in FBXO44 KD cells (FIGS. 7H-7I). FBXO44 KD increased the expression of many IFN stimulatory genes (ISGs) in RNA-seq (FIG. 8C). qRT-PCR was performed to confirm that FBXO44/SUV39H1 KD activated the expression of IFN-α/β and several ISGs, including chemokines CCL5, CXCL9, and CXCL10, by qRT-PCR (FIGS. 7E, 2B, and 8B).

To confirm RNA-seq result, ELISA was performed to quantify IFN-β, CCL5, and CXCL10. ELISA results showed that FBXO44/SUV39H1 KD enhanced the secretion of IFN-β, CCL5, and CXCL10 (FIG. 7J), which promote intratumoral infiltration of effector T cells.

To investigate the effect of FBXO44 targeting on induction of antiviral pathway and ISGs, ChIP-seq was performed as previously described. ChIP-seq results showed that none of the protein-coding genes bound by FBXO44 and harboring H3K9me3 modifications were antiviral genes or ISGs, indicating the induced antiviral pathways and ISGs were indirect effects of FBXO44 targeting. In support of these data, transfection of cytosolic dsRNA or dsDNA isolated from FBXO44 KD cells activated IFN-β expression in recipient cells (FIG. 7F).

### Example 13: Identification of FBXO44 and Interferon (IFN) Signaling Pathway

Next, antiviral pathways in inducing IFN in FBXO44 KD cells were investigated. shRNA and lentiviral transduction experiments were performed. qRT-PCR and RNA-seq experiments were performed as described in previous examples.

Cell transfection and viral transductions: plasmid transfections were performed using jetPRIME transfection reagent (Polyplus) for HEK293T and HEK293FT cells and Lipofectamine 2000 (Thermo Fisher Scientific) for all other cell lines, following the manufacturer's instructions. Lentiviruses were generated using HEK293FT cells (Thermo Fisher Scientific) and vectors pLenti CMV Puro DEST (Addgene plasmid no. 17452), pLiX 402 (Addgene plasmid no. 41394), or pLKO.1 (Addgene plasmid no. 10878) using standard techniques. Single and multiple viral transductions were performed in the presence of 8 μg/mL polybrene (Sigma-Aldrich). Lentiviral shRNA vectors for human cells included non-targeting control (Dharmacon no. RHS6848), FBXO44 #1 (Dharmacon no. RHS3979-201785935), FBXO44 #2 (Dharmacon no. RHS3979-201789150), non-targeting control (Sigma no. SCH002), LSD1 #1 (Sigma no. TRCN0000046071), LSD1 #2 (Sigma no. TRCN0000382249), MAVS (Sigma no. TRCN0000236031), and STING (Sigma no. TRCN0000163296). Lentiviral shRNA vectors for mouse cells included non-targeting control (Dharmacon no. RHS6848), FBXO44 (Dharmacon no. RMM3981-201914287), SUV39H1 (Dharmacon no. RMM3981-201816562), non-targeting control (Sigma no. SCH002), MAVS (Sigma no. TRCN0000124769), and STING (Sigma no. TRCN0000346320).

FBXO44 KD induced the expression of IFN-α/β in cancer cell lines that failed to activate either MAVS or STING, indicating stimulation of either dsRNA or dsDNA sensing pathway was sufficient to trigger the antiviral response (FIG. 2B). Experiments from shRNA lentiviral transduction showed that co-targeting of MAVS+STING in FBXO44 KD cells rescued the induced IFN-β (FIG. 7K). Similarly, simultaneous targeting of IRF3+IRF7 rescued the induced IFN-β in FBXO44 KD cells more efficiently than KD of IRF3 or IRF7 alone (FIG. 8D). Taken together, these findings demonstrate that FBXO44/SUV39H1 inhibition promotes cytosolic accumulation of dsRNA and dsDNA that trigger RIG-I/MDA5-MAVS and cGAS-STING antiviral pathways and IFN signaling in cancer cells.

GSEA analysis of FBXO44 KD cellular RNA-seq data identified several upregulated immune-related gene expression pathways, including cytokine-cytokine receptor interaction and antigen processing cross presentation (FIG. 7L). FBXO44 KD increased expression of IFNGR1 and IFNGR2, which encode the IFN-γ receptor, and decreased IFN-γ signaling inhibitor PTPN2, indicating potentially augmented responses to immune cell-derived IFN-γ (FIG. 7E).

### Example 14: Identification of FBXO44 and Cancer Cell Immunogenicity In Vitro

Since FBXO44 KD associated with enhanced antigen processing and presentation, the effects on cancer cell immunogenicity related to FBXO44 KD were investigated. RNA-seq, IF, and flow cytometry experiments were performed as described in previous examples. For IF, the relevant primary antibodies diluted in blocking buffer were then added. Specifically, 1:50 dilution for anti-ULBP2 (Santa Cruz no. sc-53135), and 1:100 dilution for anti-SSX1 (Novus Biologicals no. NBP2-00614). For flow cytometry, cell surface expression of ULBP2 and SSX1, MDA-MB-231 cells were transfected with non-targeting or FBXO44 siRNAs and stained with anti-ULBP2 antibody (GeneTex no. GTX53048) at 1:50 dilution or anti-SSX1 antibody (Novus Biologicals no. NBP2-00614) at 1:50 dilution and analyzed on a LSR Fortessa instrument (BD PharMingen).

RNA-seq results showed that FBXO44 KD induced ˜40 cancer/testis antigens (CTAs) (FIG. 7M), including MAGE-A and SSX family members that are immunotherapy targets in human cancers. IF results showed that FBXO44 KD increased SSX1 protein expression (FIG. 8E). In orthogonal experiments, FBXO44 KD enhanced SSX1 surface expression on flow cytometry (FIG. 8F). Similarly, FBXO44 KD cells increased expression of several natural killer group 2D (NKG2D) ligands, including ULBP2, which was enhanced on the cell surface (FIGS. 7M, 8E-8F). Thus, FBXO44 inhibition promotes cancer cell immunogenicity in vitro.

### Example 15: Identification of FBXO44 KD and its Effects in Cancer Cell Growth, Proliferation, and Migration

To investigate the effect of FBXO44 KD on cell proliferation and apoptosis in cancer cell lines, cancer cell lines were used and cultured as described in previous examples. Growth curve over 5 days of cancer cells was investigated. Flow cytometry to detect apoptosis was performed. Flow cytometry data analysis and RNA-seq were performed as described previously.

Flow cytometry to detect apoptosis: apoptotic cells were detected using the FITC Annexin V Apoptosis Detection Kit I (BD Biosciences no. 556547) according to the manufacturer's protocol. Cells were analyzed on an LSR Fortessa instrument (BD PharMingen).

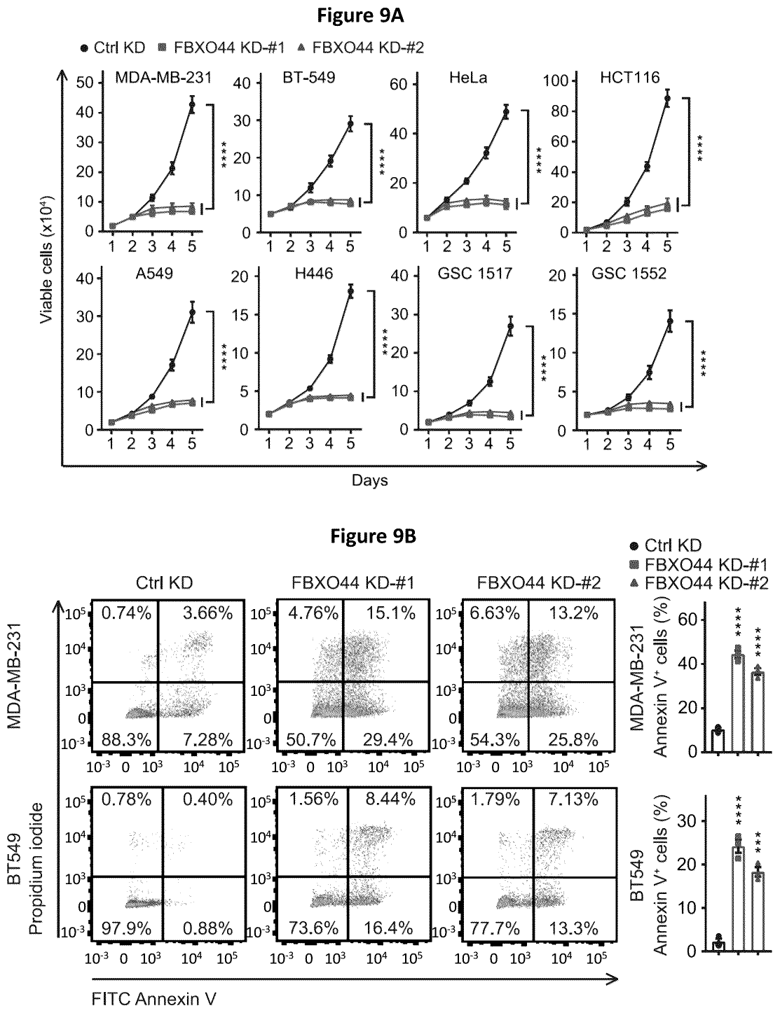

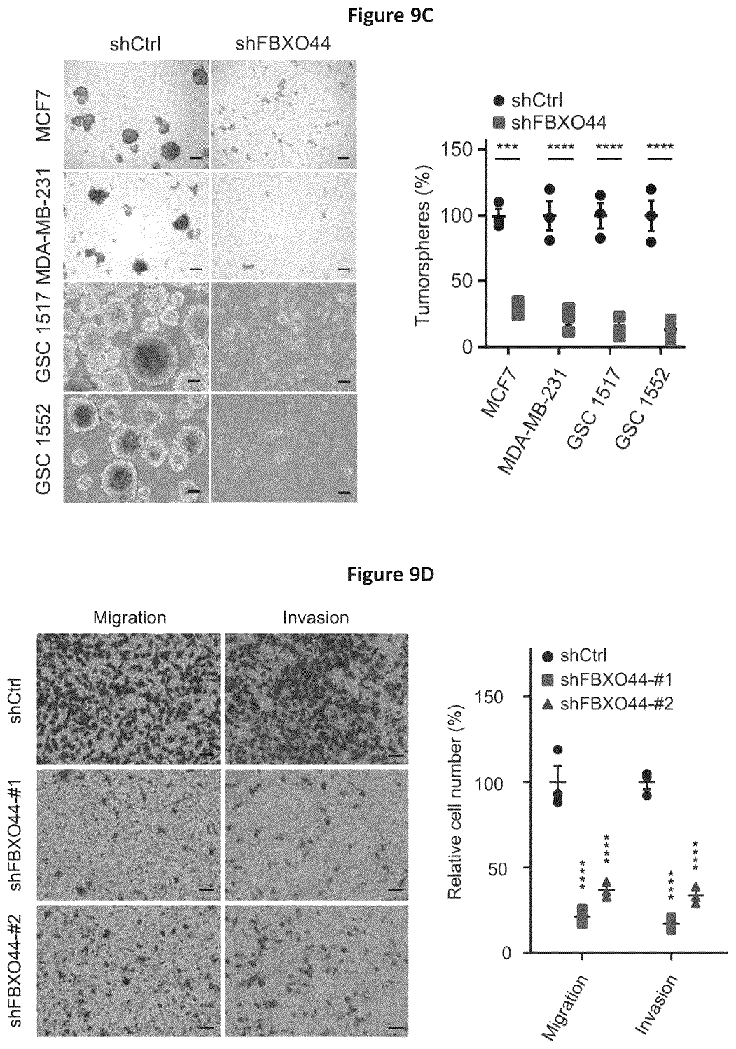

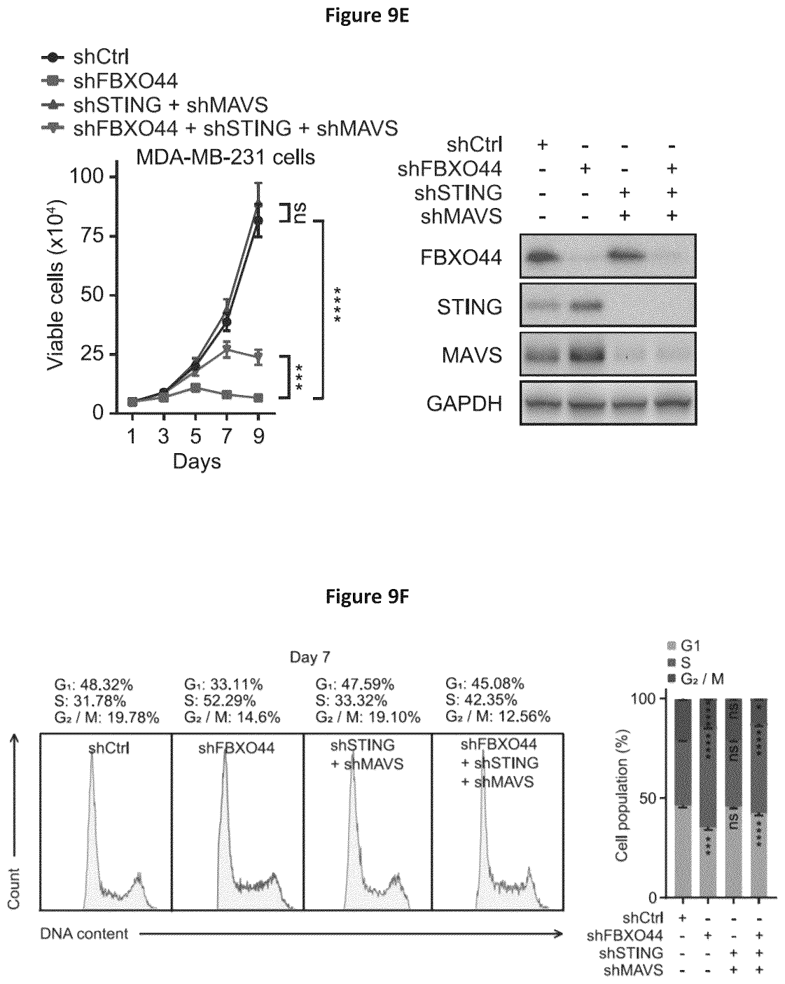

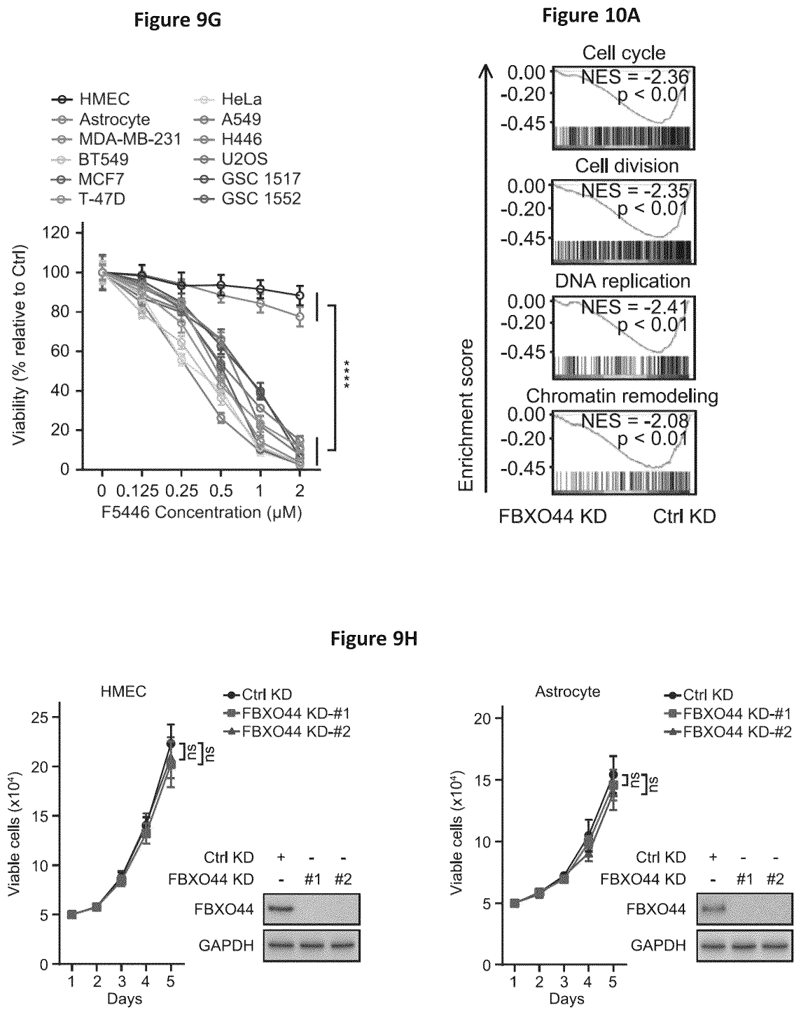

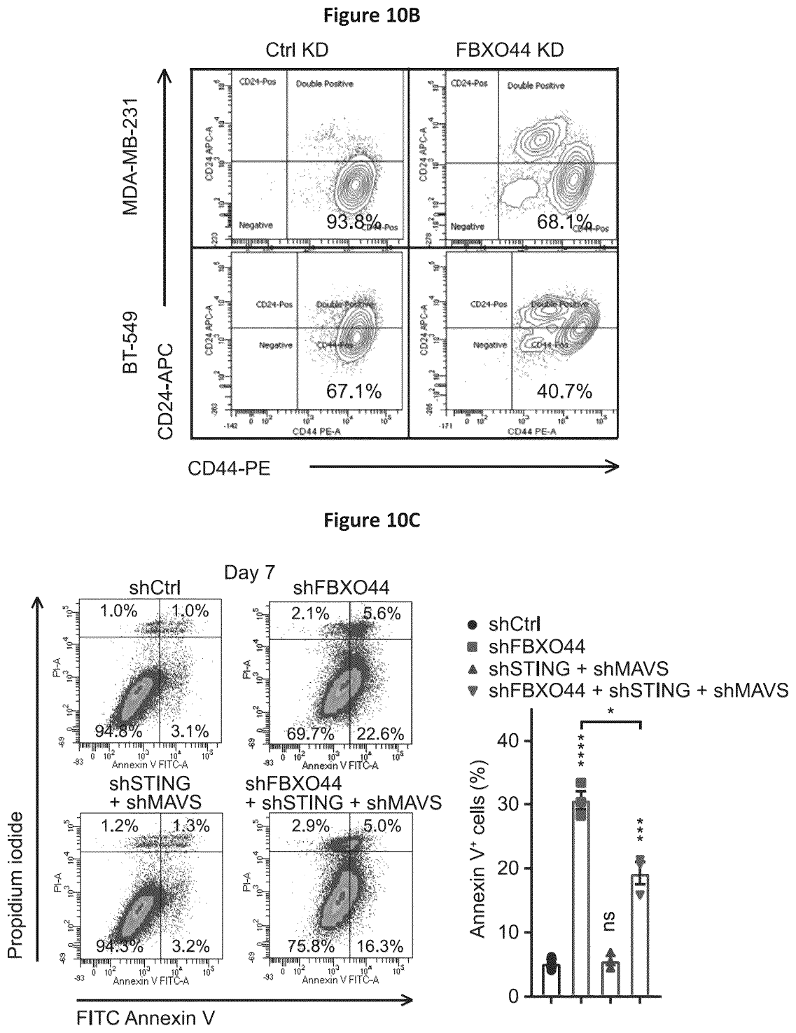

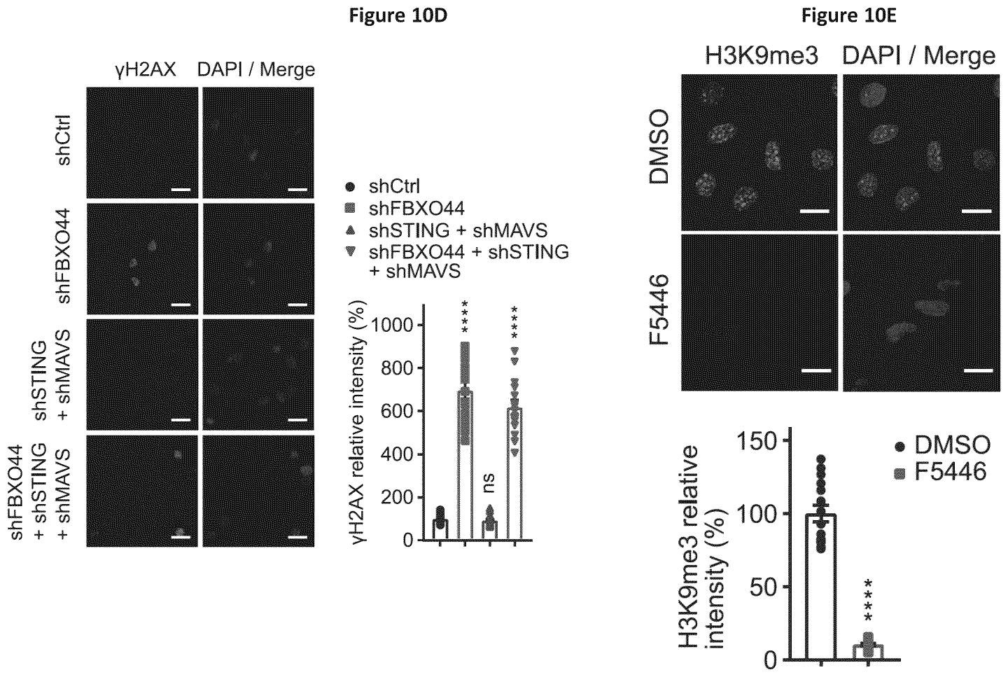

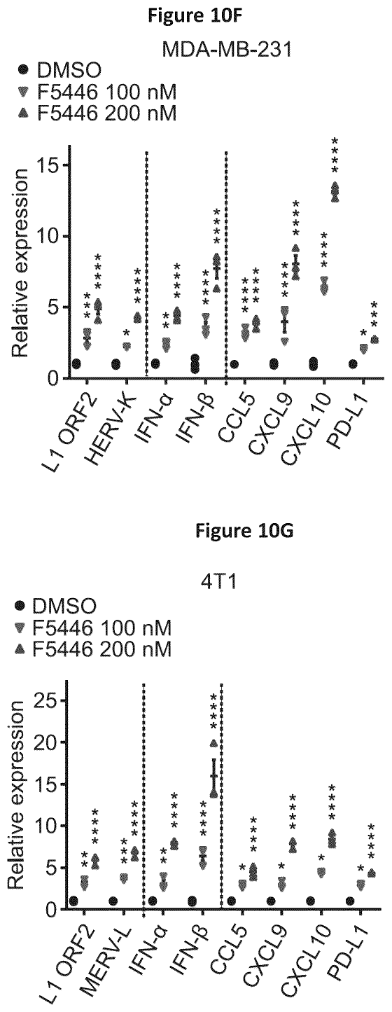

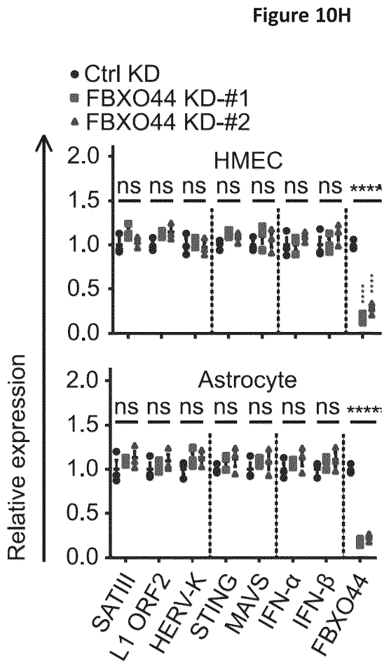

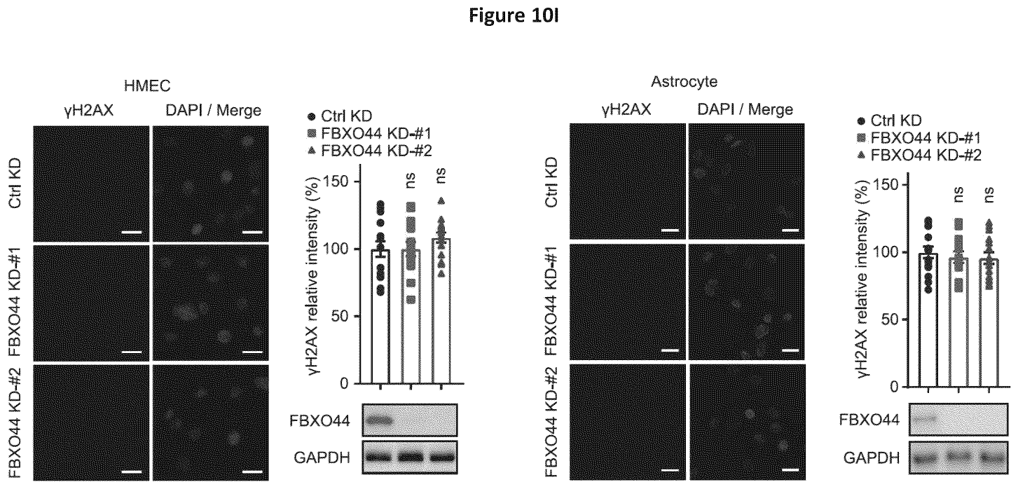

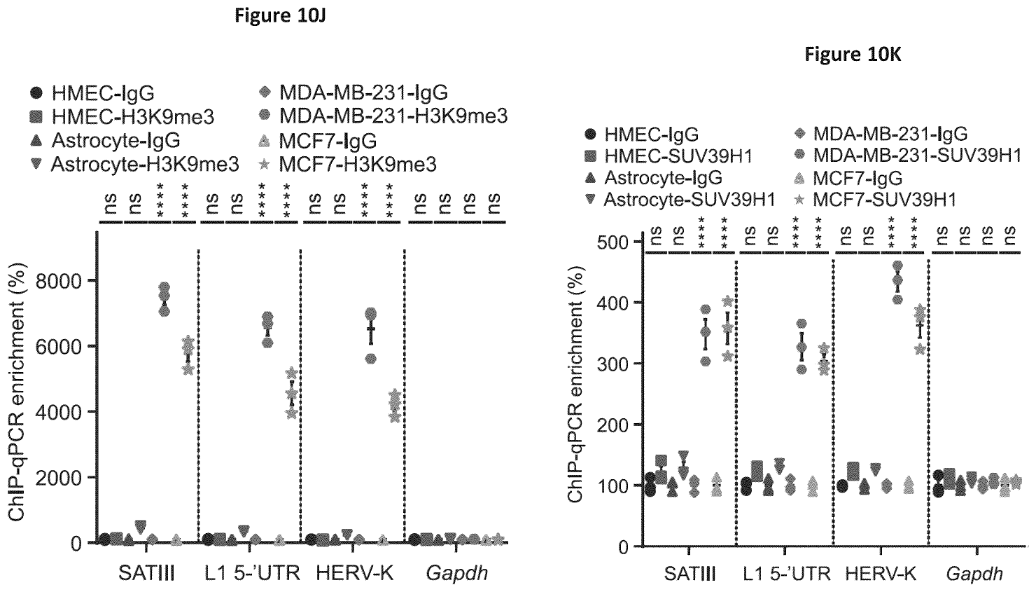

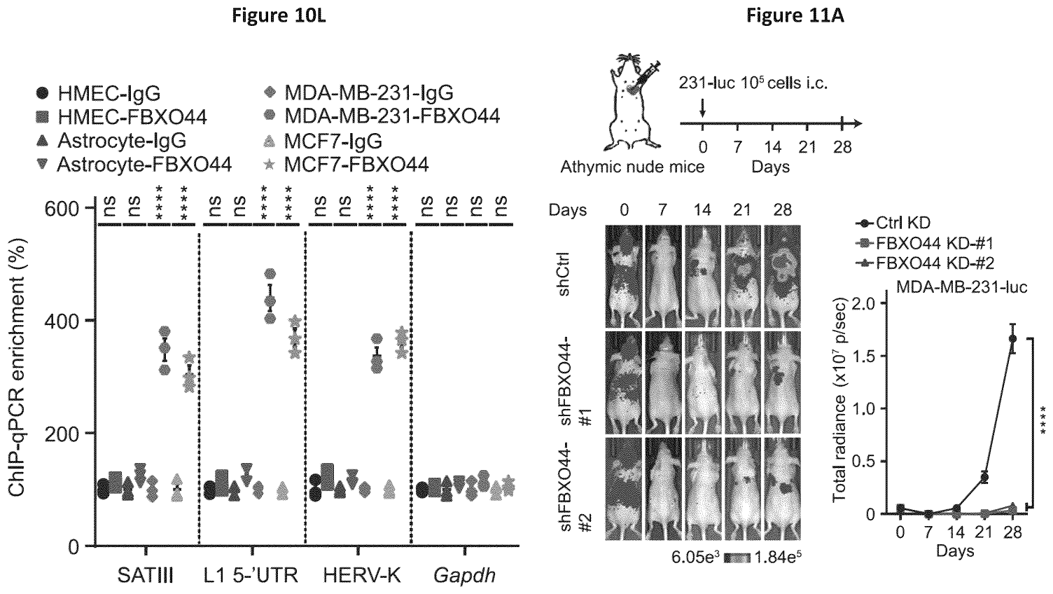

Activation of IFN signaling and genomic instability can promote cellular proliferation arrest and apoptosis. Results from cancer cell lines showed that, in preclinical studies, efficient FBXO44 KD diminished the proliferation (FIG. 9A), including patient-derived glioblastoma cultures (GSC1517 and GSC1552) enriched in cancer stem cells (CSCs), accompanied by increased apoptosis as shown by flow cytometry (FIG. 9B). Consistent with reduced proliferation, FBXO44 KD downregulated cancer-associated gene expression pathways, including cell cycle, cell division, and DNA replication, on RNA-seq (FIGS. 7H and 10A).

In order to further investigate the effect of FBXO44 KD, tumorsphere and cell migration/invasion experiments were performed. shRNA experiment was performed as described previously. Flow cytometry was performed for CSC quantifications.

Flow cyctometry for breast CSC quantification: cells were stained with anti-CD24-APC and anti-CD44-PE antibodies using the Breast Cancer Stem Cell Isolation Kit (MagCellect no. MAGH111). Cells were analyzed on a NovoCyte 3000 flow cytometer (ACEA Biosciences).

Tumorsphere assays: for MCF7 and MDA-MB-231 tumorsphere assays, 2.5×103 cells expressing the indicated shRNAs were plated into 6-well ultra-low attachment plates (Corning no. 3471) containing 500 μL of complete MammoCult Human Medium (STEMCELL Technologies no. 05620). Tumorspheres were counted after 10 days using a light microscope. For GSC tumorsphere assays, 1×105 cells expressing the indicated shRNAs were plated into regular 6-well plates containing Neurobasal medium supplemented with B27 without vitamin A, EGF, bFGF, sodium pyruvate, and GlutaMAX. Tumorspheres were counted 6 days after plating for Extreme Limiting Dilution Analysis (http://bioinf.wehi.edu.au/software/elda/). All experiments were performed in triplicate.

Cell migration/invasion assays: for migration assays, 4×104 MDA-MB-231 cells in 40 μl of medium containing 0.5% FBS were plated into the upper chamber of 24-well inserts with 8 μm pores (Trevigen no. 3484-024-01). Bottom wells contained 360 μL of medium supplemented with 10% PBS. After 24 hr, cells were fixed with cold methanol for 20 min. Non-migrating cells that remained in the upper chamber were gently removed using a cotton swab and cells that migrated to the bottom chambers were stained with 1% crystal violet. Invasion assays were performed using well inserts pre-coated with basement membrane (medium density). For both experiments, cells that migrated to the bottom chambers were quantified by imaging 4 randomly selected fields.

Results showed that FBXO44 KD decreased the fraction of breast cancer cells that expressed CSC markers (CD24− CD44+) shown by flow cytometry and inhibited tumorsphere formation of breast cancer and glioblastoma cells (FIGS. 9C and 10B). FBXO44 KD decreased cancer cell migration and invasion in vitro (FIG. 9D).

### Example 16: Identification of FBXO44 KD and Effects of MAVS+STING Co-Targeting

To investigate if co-KD of MAVS+STING can rescue effects of FBXO44 KD, growth curve, immunoblotting, shRNA, flow cytometry, and IF experiments were performed as described in previous examples.

Results showed that co-targeting of MAVS+STING partially rescued the decreased proliferation of FBXO44 KD cells in vitro (FIG. 9E). In addition, the S phase accumulation and decreased viability of FBXO44 KD cells were partially rescued by co-KD of MAVS+STING (FIGS. 9F and 10C). In contrast, co-KD of MAVS+STING failed to rescue the induced γH2AX in FBXO44 KD cells (FIG. 10D).

### Example 17: Identification of SUV39H1 Inhibitor, F5446, as a Potential Target for Cancer Treatment

To assess whether this epigenetic regulatory pathway could potentially be targeted for cancer treatment, SUV39H1 and its chemical inhibitor F5446 were used to perform experiments. IF, qRT-PCR, and growth curve experiments were performed as described in previous examples. Human mammary epithelial cells (HMECs) and primary astrocytes were also used in the experiments. HMECs (Lonza, #CC-2551) were cultured in MEGM BulletKit Medium (Lonza, #CC-3150). Primary astrocytes were cultured Astrocyte Medium (ScienCell, #1801) and cells were incubated at 37° C. in 5% CO₂. All cells were incubated at 37° C. in 5% CO₂. The cell cultures were authenticated by short tandem repeat (STR) analysis.

Results showed that F5446 treatment reduced chromatin-associated H3K9me3 (FIG. 10E) and activated the expression of various REs and IFN signaling in cancer cells (FIGS. 10F-10G), similar to SUV39H1 KD (FIG. 8B). F5446 treatment also reduced the survival of cancer cell lines and patient-derived glioblastoma cultures (FIG. 9G).

### Example 18: Identification of SUV39H1 Inhibitor, F5446, and its Effects on Normal Cells

To establish the potential of a therapeutic index for FBXO44/SUV39H1, the role of FBXO44/SUV39H1 in RE silencing in normal cells was investigated using human mammary epithelial cells (HMECs) or primary astrocytes. HMECs and primary astrocytes were cultured and treated with F5446 as described in example 17. Growth curve, immunoblotting, IF, ChIP experiments were performed as described in previous examples.

Results showed that FBXO44 KD did not affect the proliferation of HMECs or primary astrocytes (FIG. 9H), nor increased the expression of REs, MAVS/STING, or IFN-β (FIG. 10H), or increased γH2AX (FIG. 10I). In addition, F5446 treatment minimally affected HMEC and astrocyte viability (FIG. 9G). HMECs and astrocytes displayed decreased H3K9me3 levels and FBXO44/SUV39H1 binding at REs compared to cancer cell lines on ChIP (FIGS. 10J-10L), supporting cancer cell-specific effects of FBXO44/SUV39H1 inhibition on RE transcription.

### Example 19: Identification of FBXO44 Function In Vivo

To investigate the effect of FBXO44 in vivo, experiments using mouse model was performed. Immunohistochemistry (IHC) was performed to analyze mouse lung specimens,

Animal studies: mice were maintained under pathogen free conditions in 14 hrs light/10 hrs dark cycle with ad libitum access to food and water. All animal handling and procedures used were performed in accordance with protocols approved by the Institutional Animal Care and Use Committee of SBP or UCSD. For intracardiac injections, 10⁵ MDA-MB-231-luc cells transduced with lentivirus expressing non-targeting or FBXO44 shRNAs were resuspended in 100 μL DPBS and injected into the left ventricle of 6-week old female athymic nude mice (Envigo no. 069) anesthetized with isoflurane. Metastatic seeding was monitored weekly by intraperitoneal (i.p.) injection of 100 μL of 30 mg/mL D-Luciferin (Xenogen no.)CR-1001) 10 min prior to imaging using the IVIS Spectrum Xenogen Imaging System (Caliper Life Sciences). Images were analyzed using Living Image 3.0 software (Caliper Life Sciences). For ex vivo imaging of tissues, mice were injected with D-Luciferin prior to euthanasia, tissues harvested and placed in 24-well tissue culture plates containing 1 mL of 0.3 mg/mL D-Luciferin and imaged. Organs/tissues were fixed in 10% formalin and subjected to hematoxylin and eosin staining. For orthotopic injections of NSG mice, 3×10⁶ MDA-MB-231-luc cells were suspended in 100 μL of PBS and injected into the mammary fat pads of 4- to 6-week-old female mice. For F5446 drug treatments, mice were randomly divided into 3 groups at day 12. The mice were administered vehicle (10% Cremophor EL in PBS) or F5446 at doses of 10 and 20 mg/kg body weight via i.p. injection on days 12, 14, 17, 19, 21 and 24 post-inoculation of cells. Tumor size was measured every 4-5 days (long diameter and short diameter) with a caliper and tumor volume calculated as 0.5×length×width×width. Mice were imaged weekly to monitor tumor growth and metastasis in vivo using an IVIS Spectrum Xenogen Imaging System (Caliper Life Sciences). All mice were euthanized at day 25 except 3 mice were taken out from each group for tumor IHC staining of γH2AX and cleaved Caspase 3 and quantitative RT-PCR analysis at day 21.

Immunohistochemistry: For analysis of mouse lung specimens, lungs were removed and fixed in 10% formalin for 24 hr and paraffin embedded. Serial sections cut at 5 μm thickness were stained with hematoxylin (Leica no. 3801560) for 4 min followed by eosin (Leica no. 3801600) for 30 sec. Slides were then dehydrated, cleaned, and mounted with Cytoseal 60 mounting media (Thermo Fisher Scientific no. 8310-4). Tissue sections were scanned using Aperio ScanScope AT2.

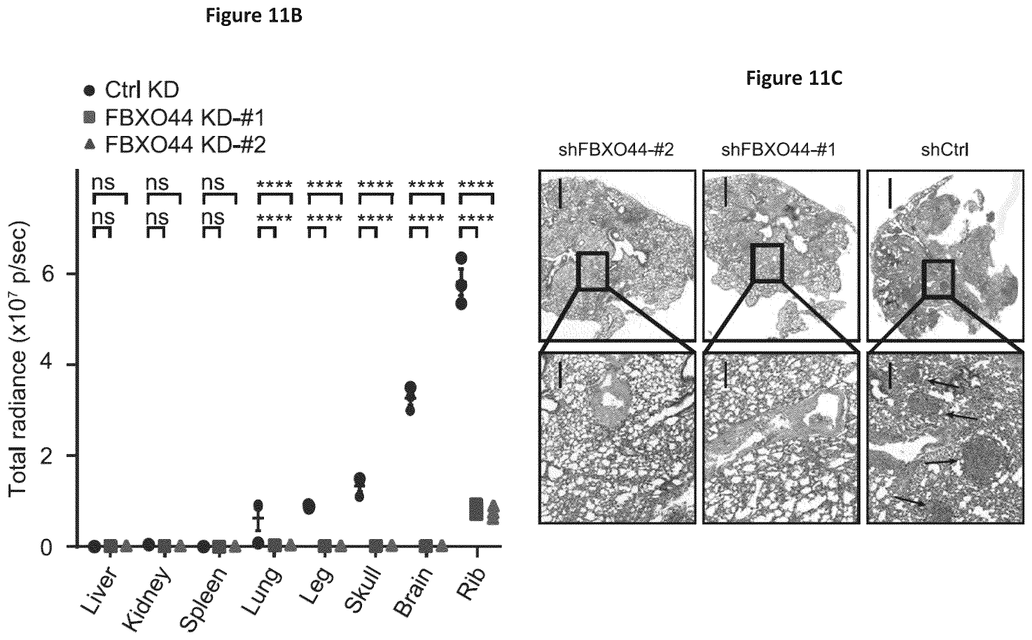

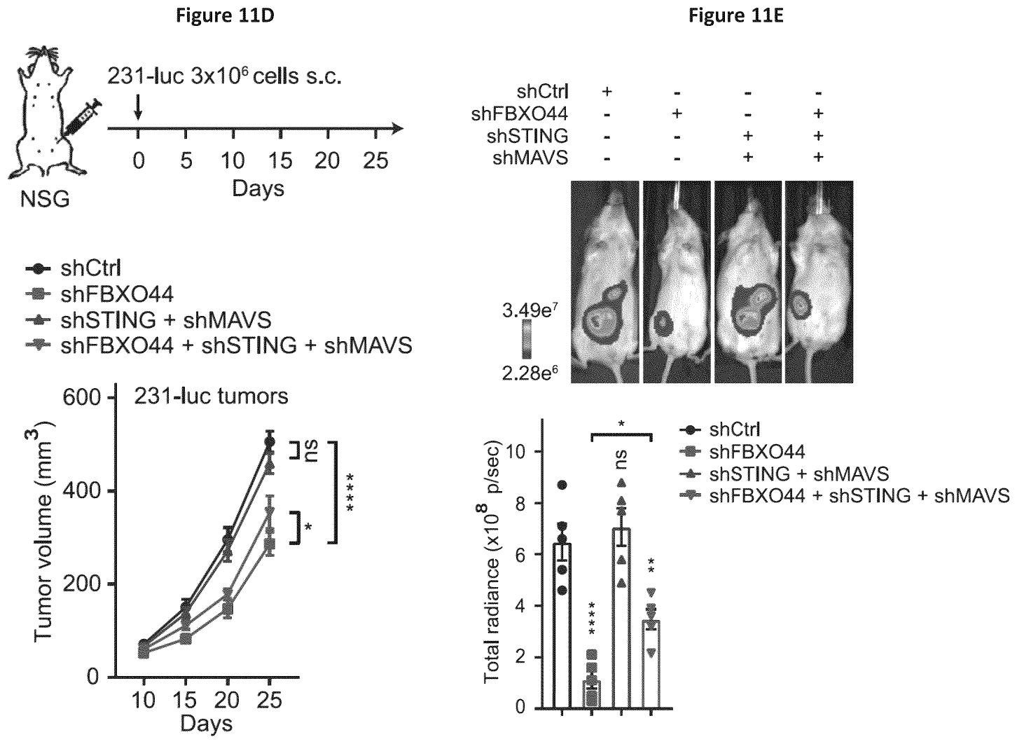

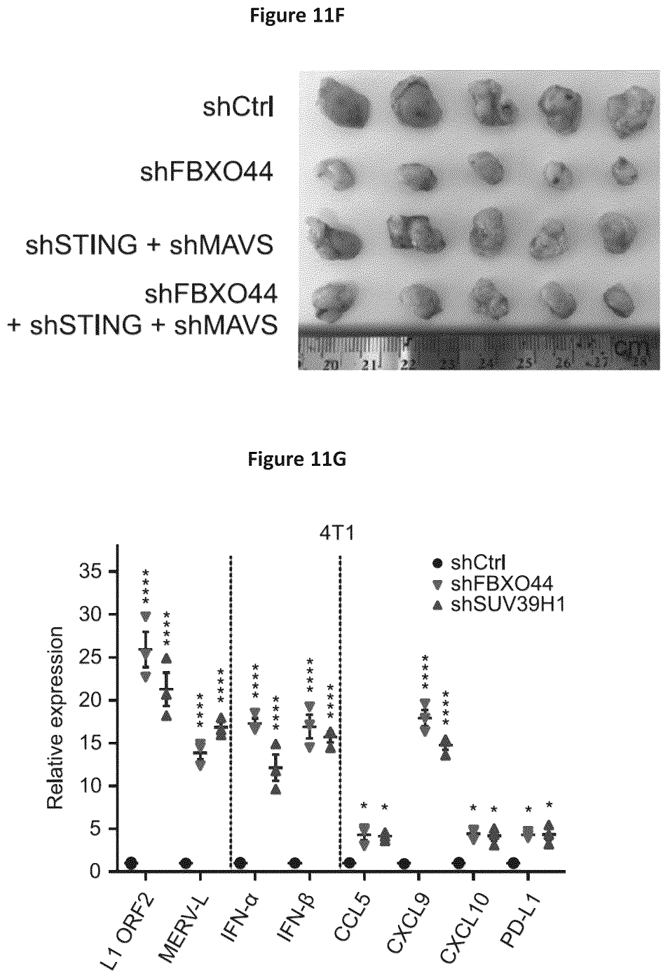

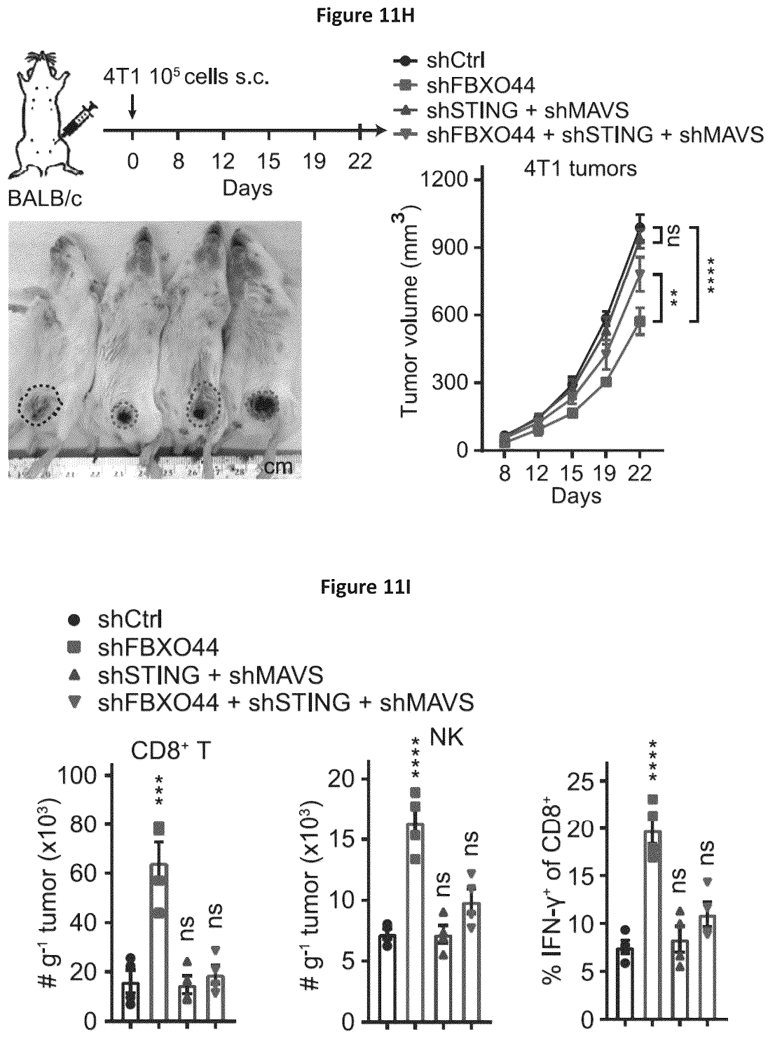

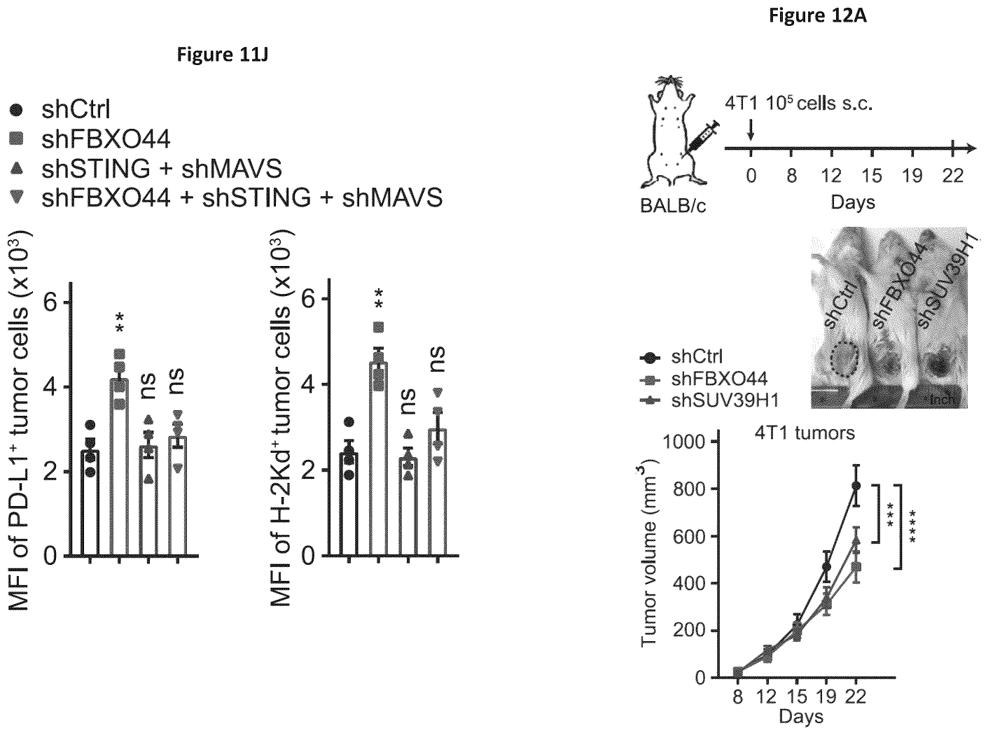

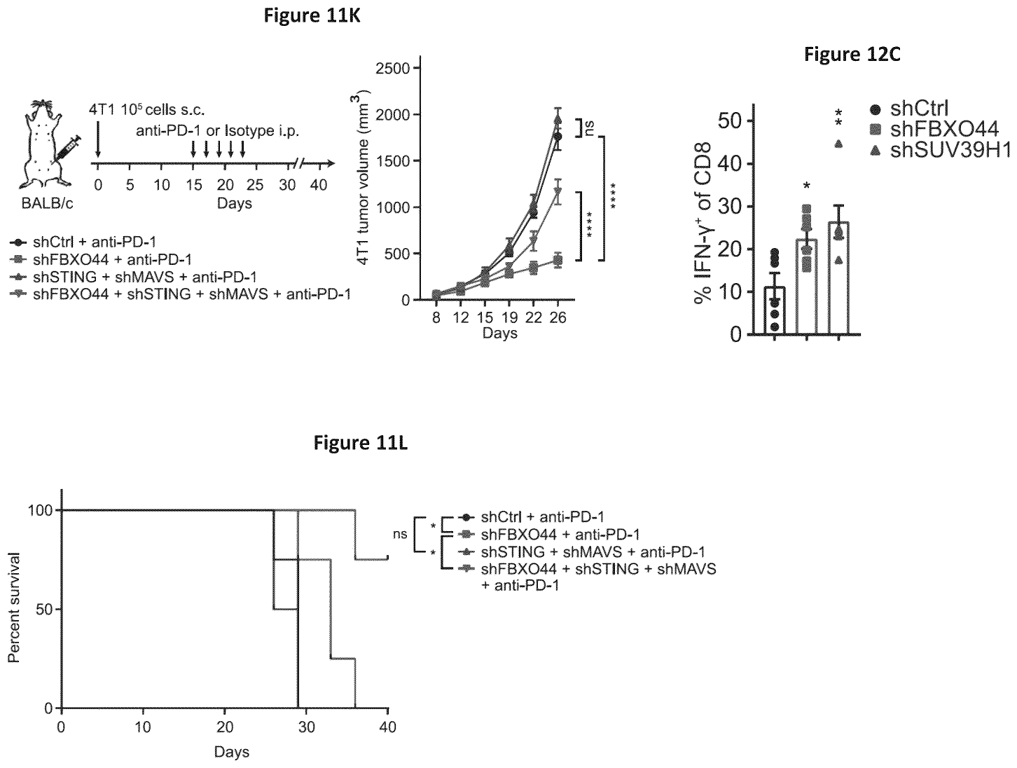

Results showed that FBXO44 KD decreased metastatic seeding of cancer cells to the lung, brain, and bone in immunodeficient mice (FIGS. 11A-11C). FBXO44 KD also decreased orthotopic mammary tumor growth (FIGS. 11D-11F). Co-targeting of MAVS+STING only partially rescued the decreased tumor growth induced by FBXO44 KD, consistent with in vitro data showing it could not fully rescue the anti-proliferation effect of FBXO44 KD cells (FIG. 9E). Thus, FBXO44/SUV39H1 inhibition decreases cell-autonomous tumor growth in mice only partially dependent on MAVS/STING signaling.

### Example 20: Identification of FBXO44/SUV39H1 KD and Immunogenicity In Vivo

To investigate the inhibition of tumor cell-intrinsic FBXO44/SUV39H1 and influence on the tumor immune microenvironment, short hairpin RNAs (shRNAs) were applied to diminish FBXO44 or SUV39H1 expression in mouse 4T1 breast cancer cells. Inoculation of 4T1 cells was performed in mice. qRT-PCR and analysis of flow cytometry results were performed as described in previous examples. IHC experiment was performed to analyse carcinoma specimens.

4T1 tumor experiment: 10⁵ 4T1 cells were inoculated into the mammary fat pads of 6- to 8-week-old female BALB/c mice (The Jackson Laboratory). Tumor size was measured twice weekly. After 22 days, mice were euthanized and portions of each tumor processed for flow cytometry and histological analysis. For antibody treatments, mice were administered 200 μg rat IgG2a isotype control (BioXCell no. BP0089) or anti-PD-1 (BioXCell no. BP0273) antibody via i.p. injection on days 15, 17, 19, 21 and 23 post-inoculation of 4T1 cells. For combination treatments, mice were administered vehicle (10% Cremophor EL in PBS) or F5446 at 10 mg/kg body weight via i.p. injection on days 12, 14, 16, 18, 20, and 22 post-inoculation of cells and administered 200 μg rat IgG2a isotype (control) or anti-PD-1 antibody via i.p. injection on days 15, 17, 19, 21, and 23 post-inoculation of 4T1 cells. Mouse survival was monitored with tumor volume exceeding 2000 mm³, weight loss>20%, and decreasing behavioral conditions considered as endpoints.

Immunohistochemistry (IHC): for MDA-MB-231-luc and 4T1 tumors, staining was performed as above using the BOND RX automated IHC system (Leica) with anti-cleaved Caspase 3 (Cell Signaling Technology no. 9664; 1:300 dilution), anti-γH2AX (Cell Signaling Technology no. 80312; 1:200 dilution), anti-CD45 (BD no. 550539; 1:50 dilution), anti-CD8a (Thermo Fisher Scientific no. 14-0808-82; 1:50 dilution), and NK Cell Marker (ANK61) (Santa Cruz no. sc-59340; 1:50 dilution) antibodies.

Flow cytometry: for 4T1 experiments, tumors were dissociated into single cells using the gentleMACS Octo Dissociator with Heaters (Miltenyi Biotec) as described in the protocol for the Tumor Dissociation Kit (Miltenyi Biotec). Cells were passed through a 70 μm filter to remove clumps and maintain single cell suspensions. Cell surface staining was performed with the indicated antibodies before fixation and permeabilization of cells for intracellular staining. For IFNγ detection, Cell Activation Cocktail (BioLegend) was used for stimulation (5 hr) prior to cell surface staining. All antibodies (anti-CD45.2, anti-CD3 anti-CD4, anti-CD8, anti-CD25, anti-CD335, anti-FOXP3, anti-IFN-γ, anti-H-2Kd, anti-PD-L1) were purchased from BioLegend.

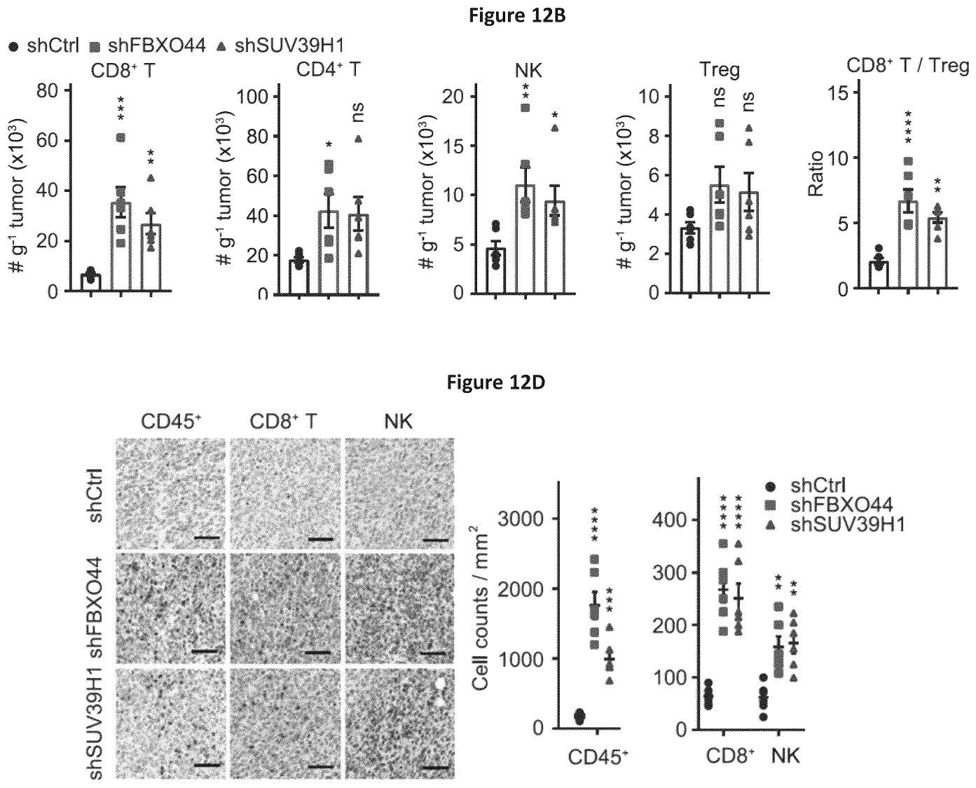

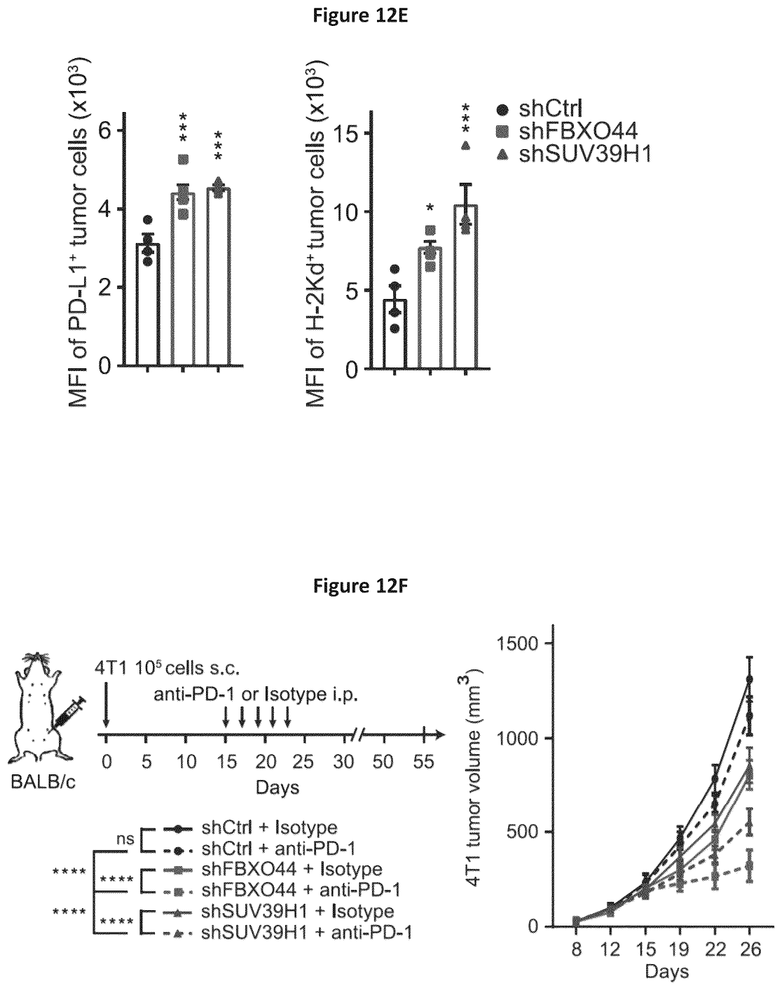

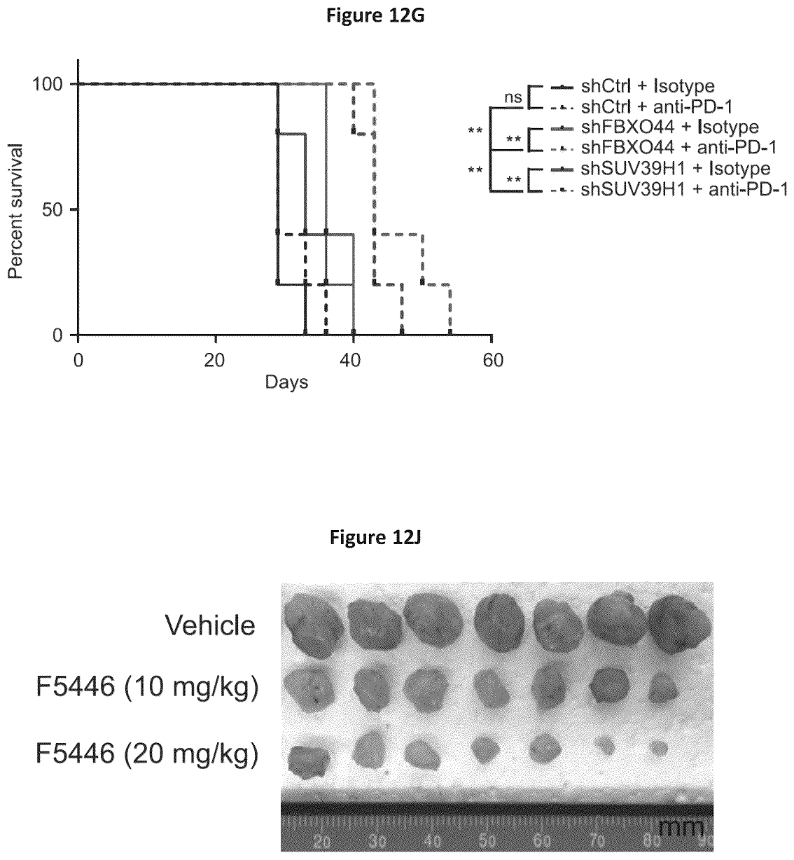

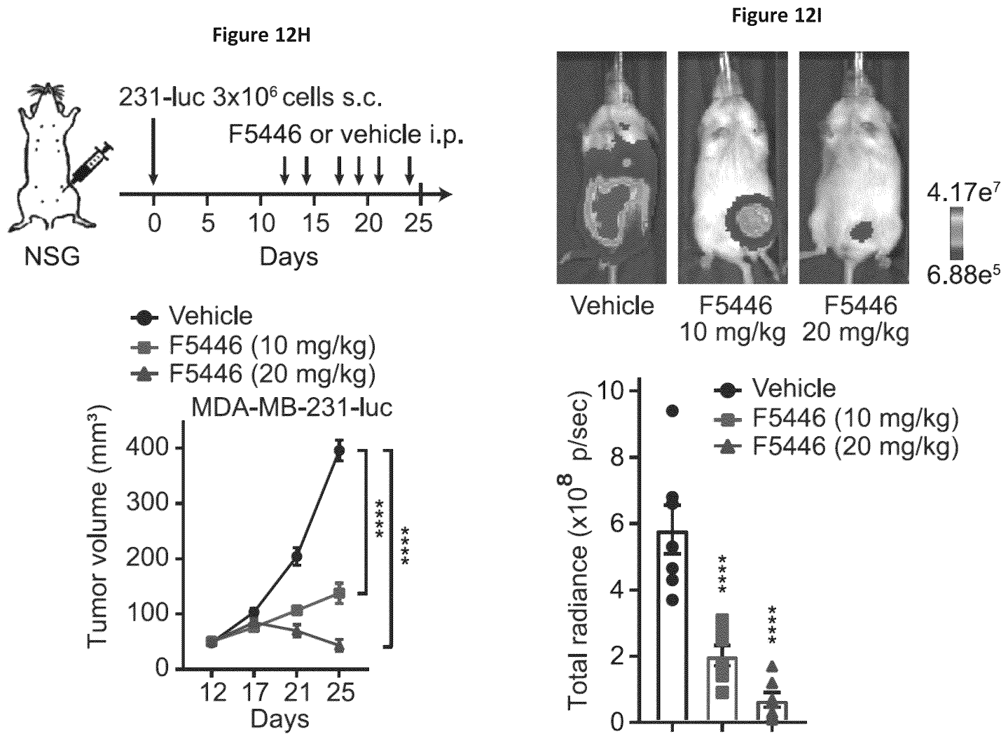

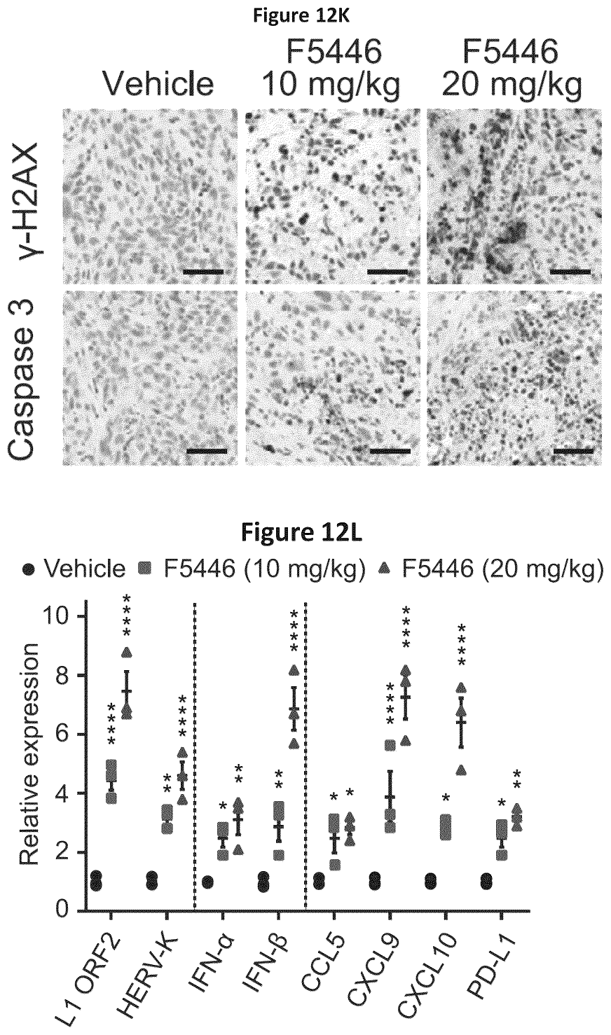

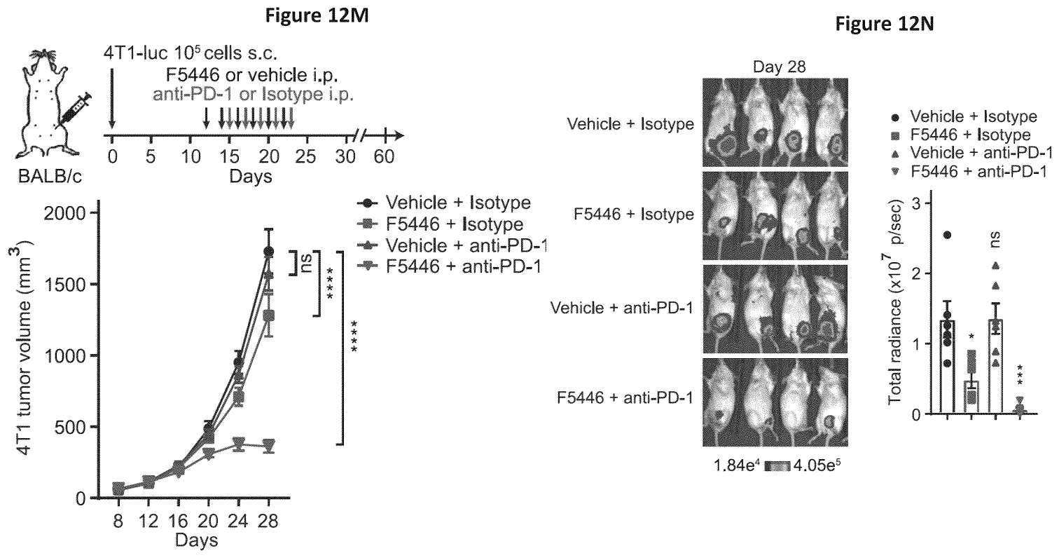

Results showed that applying short hairpin RNAs (shRNAs) diminished FBXO44 or SUV39H1 expression in mouse 4T1 breast cancer cells to levels that activated REs, IFN-α/β, and ISGs, but permitted tumor growth, thus mimicking pharmacological inhibition in cancer treatment (FIGS. 12A and 11G). Flow cytometry was performed as described in previous examples to quantify infiltrating immune cells in tumors and results showed that FBX044/SUV39H1 KD increased the intratumoral abundance of CD8+ T and natural killer (NK) cells (FIG. 12B). The CD8+/Treg cell ratio and number of IFN-γ+CD8+ T cells also increased in FBX044/SUV39H1 KD tumors, indicating a functional immune response (FIGS. 12B-12C).

IHC results showed enhanced infiltration of CD8+T and NK cells in FBX044/SUV39H1 KD tumors (FIG. 12D). FBX044/SUV39H1 KD also enhanced PD-L1 and H-2Kd MEW class I alloantigen surface expression on 4T1 tumor cells in vivo (FIG. 12E), corroborating in vitro qRT-PCR data (FIGS. 7E, 2B, 8B, 10F-10G, and 11G), demonstrating enhanced immunogenicity in vivo and potentially circumventing a major pathway of cancer cell immune evasion, consistent with other studies using DNA methylation or LSD1 inhibitors.

### Example 21: Identification of FBXO44 KD and Effects of MAVS+STRING Co-Targeting In Vivo

To investigate the effect of in FBXO44 KD, shRNA experiment was performed in mouse model as described in previous examples. Flow cytometry was performed as described in previous examples to investigate immune cells infiltration of tumor cells.

Co-targeting of MAVS+STING rescued the increased intratumoral infiltration of CD8+, NK, and IFN-γ+CD8+ cells and enhanced PD-L1 and H-2Kd MEW class I alloantigen surface expression on tumor cells induced by FBXO44 KD (FIGS. 11H-11J), consistent with in vitro data showing the induced IFN-β expression in FBXO44 KD cells was dependent on MAVS+STING (FIG. 7K).

### Example 22: Identification of FBXO44/SUV39H1 Inhibition and Enhancement in Immunogenicity and ICB Therapy

FBXO44 inhibition induced IFN signaling, enhanced cancer cell immunogenicity, and increased intratumoral infiltration of CD8+ T cells; phenotypes associated with a favorable response to immune checkpoint blockade (ICB) therapy. To investigate the effect of targeting tumor cell-intrinsic FBXO44/SUV39H1 on resistance to anti-PD-1 therapy, experiment using mouse model was performed as described previously. Immunocompetent mice bearing 4T1 cell-derived control, FBXO44 KD, or SUV39H1 KD mammary tumors were treated with either anti-PD-1 or isotype IgG control antibodies. Growth curve and survival curve experiments were performed (FIG. 12F).

Results showed that 4T1 control tumors were refractory to anti-PD-1 treatment, whereas FBXO44/SUV39H1 KD tumors displayed enhanced sensitivity. Mice bearing FBXO44/SUV39H1 KD tumors treated with anti-PD-1 therapy exhibited increased survival compared to control mice (FIG. 12G).

Co-targeting of MAVS+STING in FBXO44 KD was performed as described in previous examples to investigate the effect of anti-PD-1 therapy. Growth curve and survival curve experiments were performed. Results showed that co-targeting of MAVS+STING rescued the enhanced antitumor response to anti-PD-1 therapy and increased survival induced by FBXO44 KD (FIGS. 11K-11L). Thus, targeting tumor cell-intrinsic FBXO44/SUV39H1 enhanced immunogenicity and ICB therapy response in a MAVS/STING-dependent manner, in addition to anti-proliferative and anti-tumorigenic effects.

### Example 23: Identification of SUV39H1 Inhibitor, F5446, and its Effects on Immunotherapy Response In Vivo

To investigate the effects of pharmacologically targeting SUV39H1, F5446, on tumor growth and immunotherapy response, experiments using mouse model were performed as described in previous examples. Mice were also treated with F5446. Growth curve, survival curve, IHC, and qRT-PCR experiments were performed as described in previous examples.

Results showed that F5446 treatment inhibited mammary tumor growth in immunocompromised mice in a dose-dependent manner (FIGS. 12H-12J). F5446-treated tumors exhibited a dose-dependent increase in γH2AX and cleaved Caspase 3, a marker of apoptosis (FIG. 12K). Consistent with the observed effects in vitro (FIGS. 10F and 10G), F5446-treated tumors upregulated expression of REs, IFN α/β, and ISGs (FIG. 12L). In addition, F5446 treatment enhanced the sensitivity of 4T1 tumors to anti-PD-1 therapy, resulting in increased mouse survival (FIGS. 12M-12O).

### Example 24: Identification of FBXO44 Expression and its Association with Human Cancer

To investigate whether FBXO44 associated with human tumorigenesis and therapeutic responses, public cancer transcriptomic data were interrogated. IHC experiment was performed to detect FBXO44 level from breast specimens.

Immunohistochemistry (IHC): IHC analysis of normal breast and breast carcinoma specimens (Novus Biologicals no. NBP2-30212) were performed by antigen retrieval and blocking endogenous peroxidase activity using the BOND RX automated IHC system (Leica) followed by staining with anti-FBXO44 antibody (Sigma-Aldrich no. HPA003363) at 1:200 dilution. IHC staining was performed using the Bond Polymer Refine Detection Kit (Leica no. DS9800) and images scanned using Aperio ScanScope AT2 (Aperio Technologies). FBXO44 staining intensity was quantified using Aperio software.

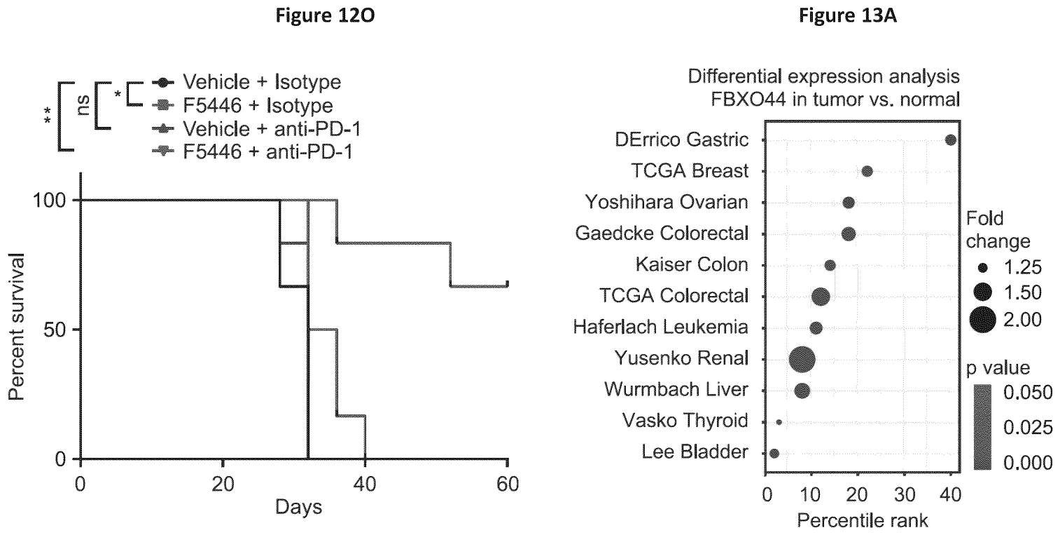

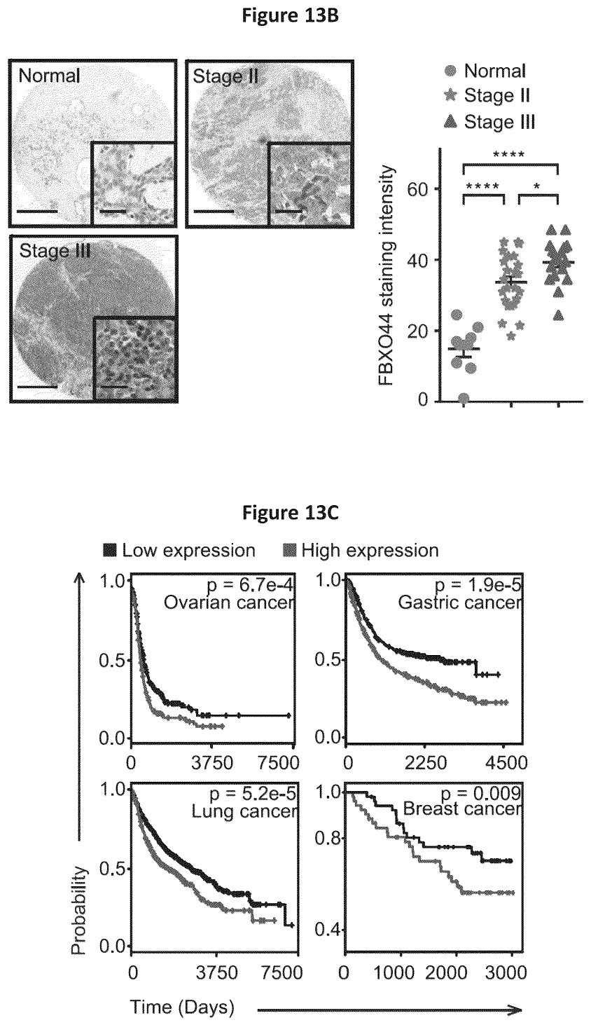

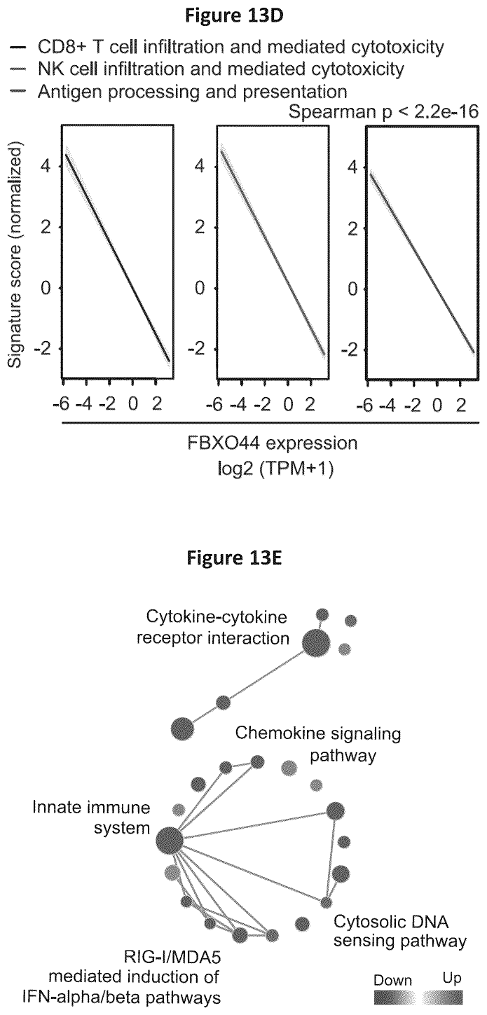

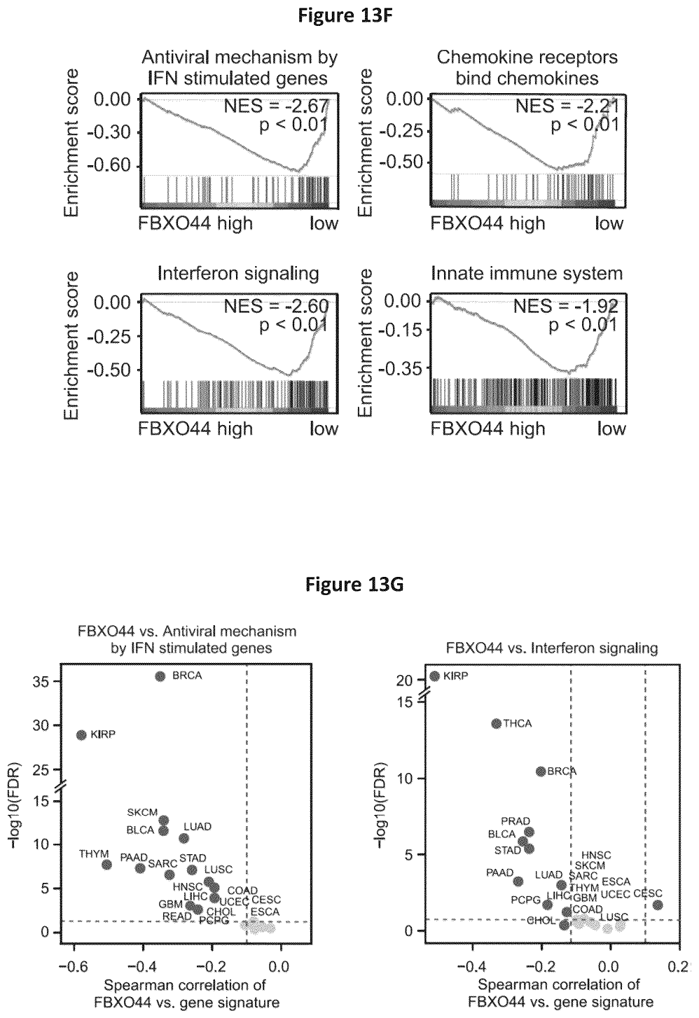

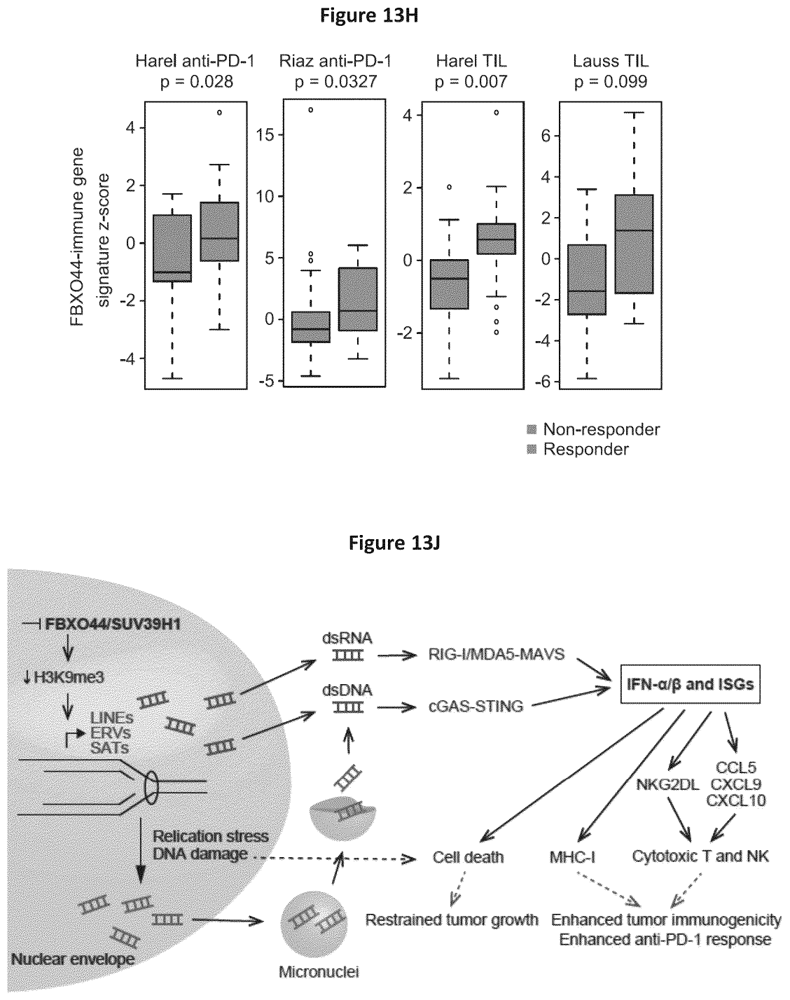

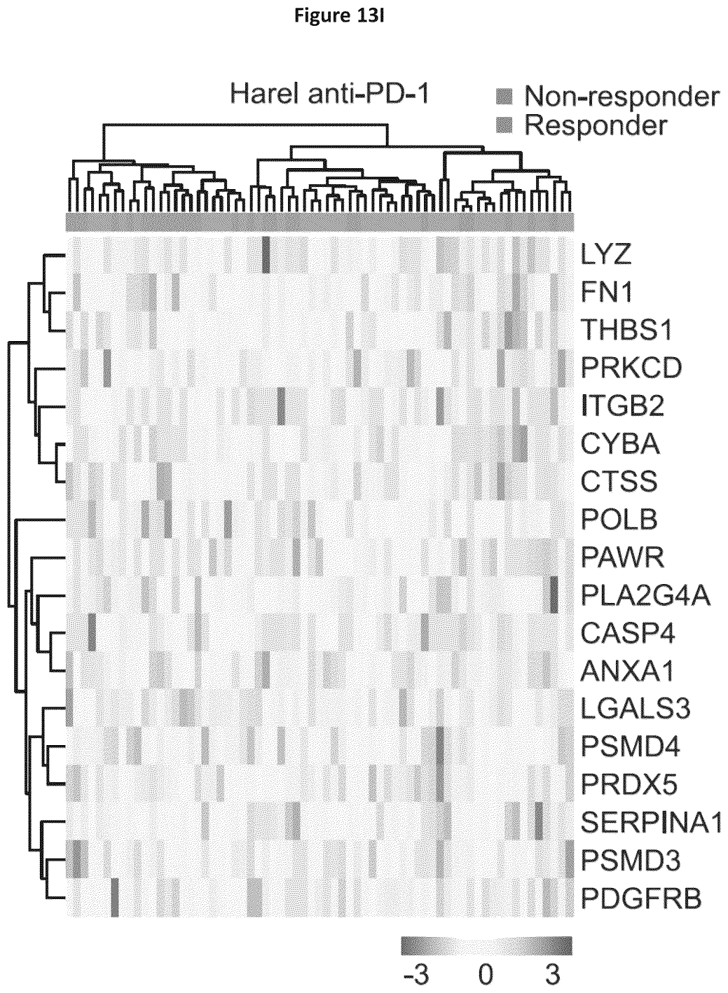

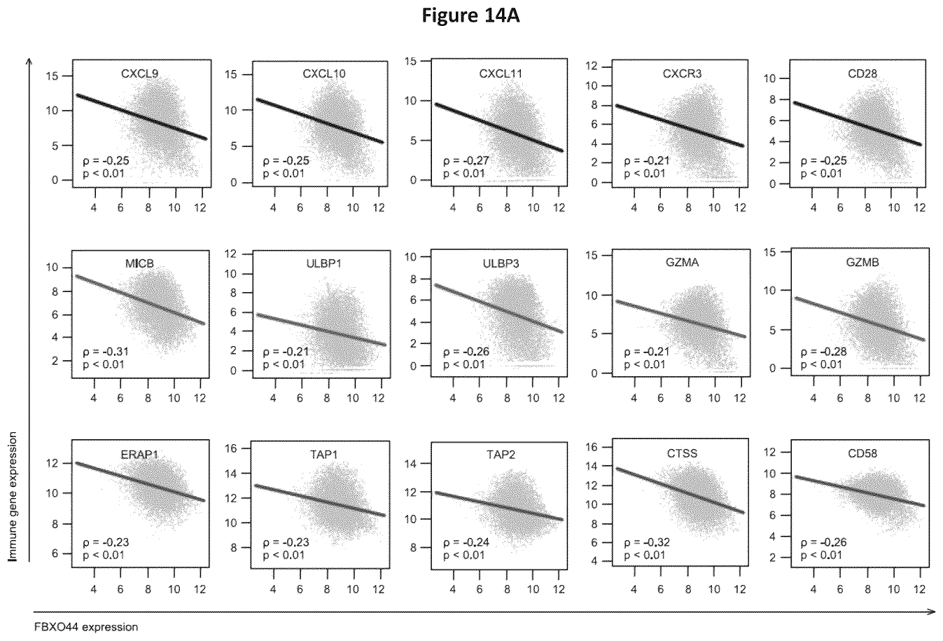

Results showed FBXO44 overexpression in many human cancer types compared to normal adjacent tissues (FIG. 13A). FBXO44 was expressed at low levels in normal breast tissues and increased with tumor stage (FIG. 13B). High FBXO44 expression correlated with poor patient outcome in several major cancer types (FIG. 13C).

### Example 25: Identification of FBXO44 Expression and Gene Expression Signatures from Cancer Genome Atlas (TCGA) Data Analysis

Next, The Cancer Genome Atlas (TCGA) was interrogated to determine if FBXO44 expression associated with the tumor immune microenvironment in cancer patients.

Pan-cancer analysis of TCGA dataset: the pan-cancer gene expression and patient annotation datasets of TCGA were retrieved from the Genomic Data Commons (GDC) of the National Cancer Institute (https://gdc.cancer.gov/). Sample-wise gene set activities of different pathways were calculated in GSVA using the “ssGSEA” method, and signature scores of different types of immune cells were calculated in GSVA using the “z-score” method. Spearman correlation and multiple testing corrections were done in R 3.5. GSEA analysis was performed using the GSEA v3.0 desktop application.

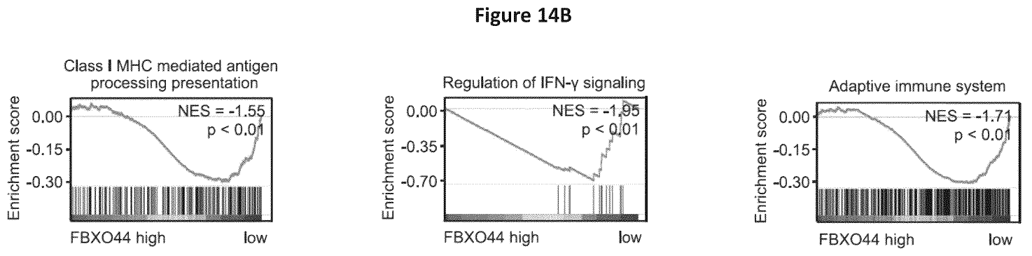

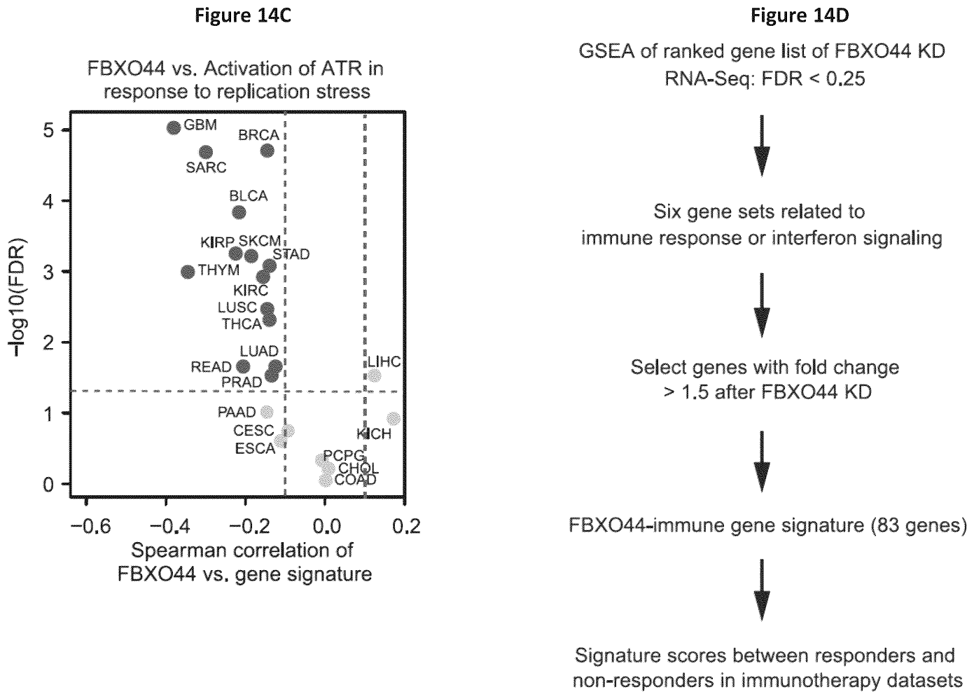

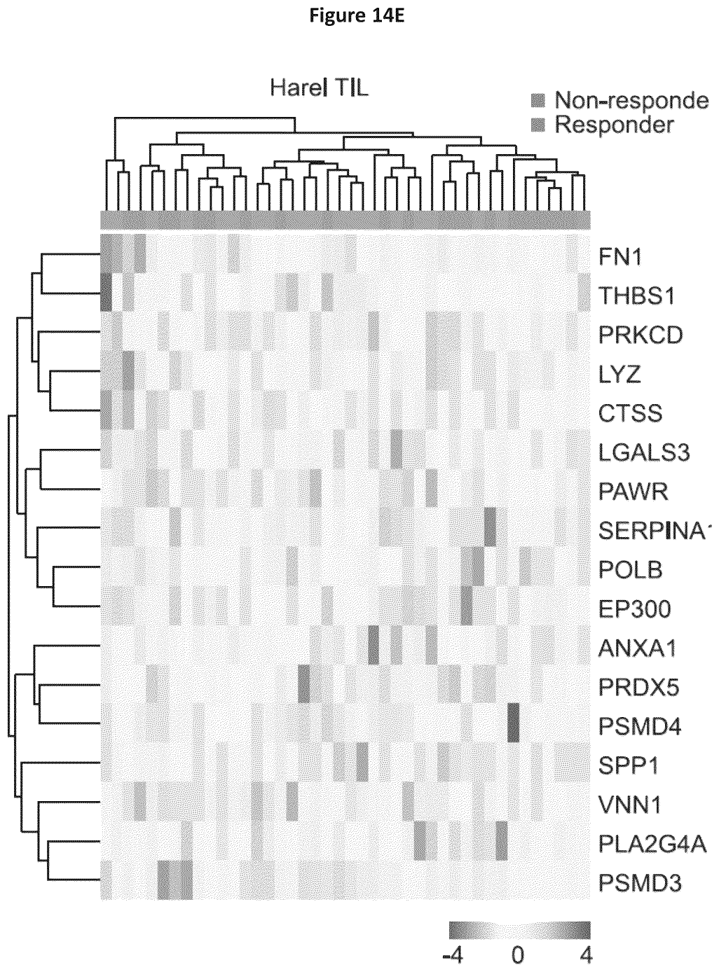

Results from pan-cancer analysis showed that FBXO44 expression inversely correlated with gene expression signatures of CD8+T and NK cell infiltration and antigen processing and presentation (FIGS. 13D, 14A). FBXO44 expression inversely correlated with innate immune system, cytosolic DNA sensing pathway, RIG-I/MDA5 mediated induction of IFN-α/β pathways, and regulation of IFN-γ signaling, among other immune-related processes (FIGS. 13E-13F, and 14B), confirming our results from in vitro and mouse models. Specifically, FBXO44 expression strongly anti-correlated with antiviral mechanism by IFN stimulated genes and interferon signaling in various human cancers (FIG. 13G). FBXO44 expression also strongly anti-correlated with activation of ATR in response to replication stress (FIG. 14C), consistent with activation of the DNA replication checkpoint in FBXO44 KD cells in vitro (FIG. 5I).

### Example 26: Identification of FBXO44-Immune Gene Signature and Response on Immunotherapy

To investigate the clinical relevance of FBXO44/SUV39H1 inhibition for cancer immunotherapy, a FBXO44-immune gene signature was analyzed and created from immunotherapy datasets.

FBXO44-immune gene signature analysis in immunotherapy datasets: the FBXO44-immune gene signature was defined as genes whose expression were upregulated at least 1.5 fold in FBXO44 knockdown RNA-seq, and also belonged to 6 sets of immune response related gene sets enriched in GSEA analysis, including “GO_INFLAMMATORY_RESPONSE”, “GO_ACTIVATION_OF_INNATE_IMMUNE_RESPONSE”, “GO_POSITIVE_REGULATION_OF_DEFENSE_RESPONSE”, “GO_POSITIVE_REGULATION_OF_LEUKOCYTE_MIGRATION”, “KEGG_CYTOKINE_CYTOKINE_RECEPTOR_INTERACTION” and “REACTOME_ANTIGEN_PROCESSING_CROSS_PRESENTATION”. The immunotherapy datasets for anti-PD1 therapy and adoptive cell transfer of TILs were analyzed from these studies. The processed protein or RNA expression data were retrieved from the publication or Gene Expression Omnibus (GSE91061 and GSE100797). The FBXO44-immune gene signature score was calculated in each dataset using the “z-score” method in GSVA. The difference of signature score between responders and non-responders were tested using unpaired Student's t-test.

From multiple public cancer immunotherapy datasets, the FBX044-immune gene signature was evaluated its predictive value, including anti-PD-1 and tumor-infiltrating T cell (TIL) therapies (FIG. 14D). Responders to immune therapies expressed higher levels of the FBX044-immune gene signature than non-responders across most of the datasets (FIGS. 13H-13I, and 14E), suggesting that FBX044/SUV39H1 inhibition sensitizes cancers to immunotherapy.

### Example 26: FBX044 and PD-1 Inhibitor Combination Therapy for Chemically Induced Squamous Skin Cancer (cSCC)

In order to show the Effect of FBX044 and PD-1 Inhibitor Combination Therapy the cSCC tumor system 168 (cultured in vitro with ≤2 passages) was injected subcutaneously. When the tumor reaches a diameter of about 3-5 mm (about 2-3 weeks after transplantation), it is treated 3 times at 4-day intervals via intraperitoneal injection (ip) (mice day 0, 4 days). Anti-PD-1 (α-PD-1) antibody alone (250 Clone: RMP1-14, Catalog No.: BE0146, BioXCell), pan, injected at eye and day 8 with n=7) per arm. Mice were treated with either specific (anti-FBX044) antibody (200 μg) alone or a mixture of α-PD-1 and FBX044 antibody. Subsequently, the size of the tumor was measured with a caliper. α-PD-1 monotherapy inhibited tumor growth compared to control mice, but tumor regression was not sustained after 12-14 days. Both α-PD-1 and α-FBX044 monotherapy induced tumor regression compared to the corresponding IgG-controlled mice, but on average the mixed effects of α-PD-1 and α-FBX044 eventually is larger than anti-PD1 reagent alone. Levels of immune cell markers in these tumors are measured as a percentage of hepatocytes and as a percentage of CD45+ cells to assess changes in the population of tumor-infiltrating immune cells in response to the drug. Fluorescent activated cells with CD45+, natural killer (NK) cells, regulatory T (Treg) cells, CD4+ and CD8+ T cells in 2-3 tumors per cohort on day 8 after the third treatment with the inhibitor is measured by sorting (FACS). The elevated CD8+cell number is utilized as a biomarker for α-FBX044 and α-PD-1 inhibitor responsiveness.

### Example 27: Tumor Size Reduction with α-FBX044/α-PD-1 Combination Therapy in Chemically-Induced (DMBA-TPA) Squamous Cell Skin Cancer (cSCC) Mice

In addition to the use of an allograft model, the antitumor effects of the α-FBX044/α-PD-1 combination therapy are also evaluated using a directly chemically induced mouse skin model of carcinogenesis. The DMBA-TPA (12-O tetradecanoyl-phorbol-13-acetate)-induced cSCC model is well characterized and is widely used to identify the mechanism of. genetic and molecular oncogenes of cancer initiation and progression that may not be achievable from simpler models such as human cancer studies or tumor allogeneic implants. This model is initiated by a DMBA mutation in Hras codon 61 in >90% of wild-type mouse tumors (Quintanilla M et al., Nature., 322 (6074): 78-80, 1986). Multiple biweekly treatments with the subsequent tumor promoter TPA stimulate the growth of benign precancerous papillomas. A percentage of papillomas progress to cancer over a period of 4-12 months, and in some cases to highly invasive spindle-shaped tumors that have lost many of their epithelial cell properties.

Using a bioluminescent reporter strain of luciferase knock-in mice (p16 (LUC)), chemically induced cSCC growth was measured before and after treatment of mice with a combination of α-PD1 and α-FBX044 antibodies. p16-LUC mice show expression of the p16 (INK4a) gene, a tumor suppressor in Ras-driven tumor cells. The reporter is activated by early neoplastic events and allows tumor visualization and tumor size measurement using the IVIS optical imaging system.

Tumors are initiated by topical treatment of 3 mice with DMBA twice the first week and with TPA for the next 20 weeks, and carcinoma growth is observed at about 30 weeks. Surgical resection of the carcinoma is performed 32 weeks after tumor initiation with DMBA treatment. Mice are imaged and tumor size measured 5 times (before surgical resection: week 0 and after surgical resection: 8, 12, 21, and 27 weeks).

When all mice had a carcinoma of approximately 15 mm and small lung metastases (3×3 mm), the carcinoma was resected and treated three times at 4-day intervals via intraperitoneal injection (ip). (Mice were injected on days 8, 12, and 16 (n=3)). Mice are treated with a mixture of α-PD-1 antibody (250 Clone: RMP1-14, BioXCell) and α-FBX044 antibody (200 μg). Tumor size is subsequently measured using a luminescent imaging device. Combination α-PD-1 and α-FBX044 antibody treatment results in tumor regression in all three mice.

Carcinomas resected from mice are classified by FACS for cell markers CD45+, natural killer (NK) cells, regulatory T (Treg) cells, CD4+ and CD8+ T cells, and in tumors after a third treatment with inhibitors. Tumors that respond to treatment with α-PD-1/α-FBX044 alone, show significant increases in CD4+ and CD8+ T cell levels. A-PD-1/α-FBX044 responsive tumors show even higher CD4+ and CD8+ T cell subsets. In addition, α-PD-1/α-FBX044 responsive tumors with and in combination of α-FBX044 alone show CD8+T effector (Teff)/T regulatory factor (Treg) CD45+ cells. The elevated CD8+ T cell number is utilized as a biomarker for α-FBX044 and α-PD-1 inhibitor responsiveness.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating a cancer in an individual in need thereof, comprising administering to the individual two components: i) an inhibitor of F-Box protein FBXO44; and ii) a cancer immunotherapy agent.

2. The method of claim 1, wherein the cancer immunotherapy agent is an immune checkpoint inhibitor, a cancer vaccine, or a cytokine therapy agent.

3. The method of claim 2, wherein the immune checkpoint inhibitor is a chemical entity that blocks PD-1, PD-L1, B7-1, B7-2 or CTLA-4.

4. (canceled)

5. The method of claim 3, wherein the immune checkpoint inhibitor is a chemical entity that blocks PD-1.

6. The method of claim 5, wherein the chemical entity that blocks PD-1 is an anti-PD-1 antibody.

7. The method of claim 6, wherein the anti-PD-1 antibody is BioXCell Catalog No. BP0273, BE0273, BP0146, BE0146, BP0033-2, BE0033-2, or a combination thereof.

8. The method of claim 6, wherein the anti-PD-1 antibody is Pembrolizumab, Nivolumab, Cemiplimab, or a combination thereof.

9. (canceled)

10. The method of claim 2, wherein the immune checkpoint inhibitor is a chemical entity that blocks PD-L1, and wherein the chemical entity that blocks PD-L1 is an anti-PD-L1 antibody.

11. The method of claim 10, wherein the anti-PD-L1 antibody is Atezolizumab, Avelumab, Durvalumab, or a combination thereof.

12. The method of claim 2, wherein the immune checkpoint inhibitor is a chemical entity that blocks CTLA-4.

13. The method of claim 12, wherein the chemical entity that blocks CTLA-4 is an anti-CTLA-4 antibody.

14. The method of claim 13, wherein the anti-CTLA-4 antibody is Ipilimumab.

15. The method of claim 1, wherein the cancer is breast cancer, lung cancer, gastric cancer or ovarian cancer.

16. The method of claim 1, wherein the cancer is treatment resistant.

17. The method of claim 16, wherein the individual previously was treated with another cancer immunotherapy agent.

18. (canceled)

19. A method of treating treatment resistant cancer in an individual in need thereof, comprising administering to the individual at least two components: i) a chemical entity that interferes with F-Box FBXO44 protein synthesis; and ii) a cancer immunotherapy agent.

20. The method of claim 19, wherein the chemical entity is a FBXO44 siRNA.

21. (canceled)

22. The method of claim 19, wherein the cancer immunotherapy agent is an immune checkpoint inhibitor, a cancer vaccine, or a cytokine therapy agent.

23-38. (canceled)

39. A method of treating a cancer in an individual in need thereof, comprising determining whether the cancer is resistant to treatment with a first cancer immunotherapy agent; and administering to the individual two components: i) an inhibitor of F-Box protein FBXO44 or SUV39H1; and ii) a second cancer immunotherapy agent, wherein the inhibitor of F-Box protein FBXO44 or SUV39H1 is not 1-benzyl 7-methyl 6-((4-chlorophenyl)sulfonyl)-4,5-dioxo-3,4,5,6-tetrahydropyrrolo[3,2-e]indole-1,7-dicarboxylate or 1-(4-fluorobenzyl) 7-methyl 4,5-dioxo-6-tosyl-3,4,5,6-tetrahydropyrrolo[3,2-e]indole-1,7-dicarboxylate, or a pharmaceutically acceptable salt thereof.

40. The method of claim 39, wherein the second cancer immunotherapy agent is an immune checkpoint inhibitor, cancer vaccine, or cytokine therapy agent.

41-59. (canceled)
